(19) United States
(12) Patent Application Publication
(10) Pub. No.: US 2004/0033942 A1
(43) Pub. Date: Feb. 19, 2004

(54) Transriotion factors

(76) Inventors: Jennifer L Jackson, Santa Cruz, CA (US); Mariah R Baughn, San Leandro, CA (US); Henry Yue, Sunnyvale, CA (US); Preeti G Lal, Santa Clara, CA (US); Dyung Aina M Lu, San Jose, CA (US); Chandra S Arvizu, San Jose, CA (US); Yalda Azimzai, Oakland, CA (US); Olga Bandman, Mountain View, CA (US); Y Tom Tang, San Jose, CA (US); Preete Bhanot, Bakersfield, CA (US); Purvi Shah, San Jose, CA (US); Janice Au-Yong, Brisbane, CA (US)

Correspondence Address: INCYTE CORPORATION (formerly known as Incyte, Genomics, Inc.), 3160 PORTER DRIVE, PALO ALTO, CA, 94304, US

(21) Appl. No.: 10/221,625

(22) PCT Filed: Mar. 13, 2001
(86) PCT No.: PCT/US01/08117

(51) Int. Cl. C12Q 1/68; C07H 21/04; C12N 9/22; C12P 21/02; C12N 5/06

(52) U.S. Cl. 514/12; 435/6; 435/69.1; 435/320.1; 435/325; 435/199; 536/23.2

(57) ABSTRACT

The invention provides human transcription factors (TRFX) and polynucleotides which identify and encode TRFX. The invention also provides expression vectors, host cells, antibodies, agonists, and antagonists. The invention also provides methods for diagnosing, treating, or preventing disorders associated with expression of TRFX.

### TECHNICAL FTELD

This invention relates to nucleic acid and amino acid sequences of transcription factors and to the use of these sequences in the diagnosis, treatment, and prevention of cell proliferative, autoimmune/inflammatory, neurological, and developmental disorders, and in the assessment of the effects of exogenous compounds on the expression of nucleic acid and amino acid sequences of transcription factors.

### BACKGROUND OF THE INVENTION

Multicellular organisms are comprised of diverse cell types that differ dramatically both in structure and function. The identity of a cell is determined by its characteristic pattern of gene expression, and different cell types express overlapping but distinct sets of genes throughout development. Spatial and temporal regulation of gene expression is critical for the control of cell proliferation, cell differentiation, apoptosis, and other processes that contribute to organism development. Furthermore, gene expression is regulated in response to extracellular signals that mediate cell-cell communication and coordinate the activities of different cell types. Appropriate gene regulation also ensures that cells function efficiently by expressing only those genes whose functions are required at a given time.

Transcriptional regulatory proteins are essential for the control of gene expression. Some of these proteins function as transcription factors that initiate, activate, repress, or terminate gene transcription. Transcription factors generally bind to promoter, enhancer, or upstream regulatory regions of a gene in a sequence-specific manner, although some factors bind regulatory elements within or downstream of the coding region. Transcription factors may bind to a specific region of DNA singly or as a complex with other accessory factors. (Reviewed in Lewin, B. (1990) Genes IV, Oxford University Press, New York, N.Y., pp. 554570.)

The double helix structure and repeated sequences of DNA create topological and chemical features which can be recognized by transcription factors. These features include hydrogen bond donor and acceptor groups, hydrophobic patches, major and minor grooves, and regular repeated stretches of sequence which induce distinct bends in the helix. Typically, transcription factors recognize specific DNA sequence motifs of about 20 nucleotides in length. Multiple adjacent transcription factor-binding motifs may be required for gene regulation.

Many transcription factors incorporate DNA-binding structural motifs which comprise either a helices or β sheets that bind to the major groove of DNA. Four well-characterized structural motifs are helix-turn-helix, zinc finger, leucine zipper, and helix-loop-helix. Proteins containing these motifs may act alone as monomers or form homo- or heterodimers that interact with DNA.

The zinc finger motif, which binds zinc ions, generally contains tandem repeats of about 30 amino acids consisting of periodically spaced cysteine and histidine residues. Examples of this sequence pattern include the C2H2-type and the C3HC4-type zinc fingers, and the PHD domain. (Lewin, supra; Aasland, R., et al. (1995) Trends Biochem. Sci 20:56-59.) Zinc finger proteins each contain an a helix and an antiparallel β sheet whose proximity and conformation are maintained by the zinc ion. Contact with DNA is made by the arginine preceding the a helix and by the second, third, and sixth residues of the a helix.

The leucine zipper motif comprises a stretch of amino acids rich in leucine which can form an amphipathic a helix. This structure provides the basis for dimerization of two leucine zipper proteins. The region adjacent to the leucine zipper is usually basic, and upon protein dimerization, is optimally positioned for binding to the major groove. Proteins containing such motifs are generally referred to as bZIP transcription factors. The helix-loop-helix motif (HLH) consists of a short a helix connected by a loop to a longer a helix. The loop is flexible and allows the two helices to fold back against each other and to bind to DNA. The transcription factor Myc contains a prototypical HLH motif. Most transcription factors contain characteristic DNA binding motifs, and variations on the above motifs and new motifs have been and are currently being characterized (Faisst, S. and S. Meyer (1992) Nucl. Acids Res. 20:3-26).

Mutations in transcription factors contribute to oncogenesis. This is likely due to the role of transcription factors in the expression of genes involved in cell proliferation. For example, mutations in transcription factors encoded by proto-oncogenes, such as Fos, Jun, Myc, Rel, and Spi1, may be oncogenic due to increased stimulation of cell proliferation. Conversely, mutations in transcription factors encoded by tumor suppressor genes, such as p53, RB1, and WT1, may be oncogenic due to decreased inhibition of cell proliferation. (Latchman, D. (1995) Gene Regulation: A Eukarvotic Perspective, Chapman and Hall, London, UK, pp 242-255.)

Gene expression is also affected by chromatin-associated proteins. In the nucleus, DNA is packaged into chromatin, the compact organization of which limits the accessibility of DNA to transcription factors and plays a key role in gene regulation. (Lewin, supra, pp. 409-410.) The compact structure of chromatin is determined and influenced by chromatin-associated proteins such as histones, high mobility group (HMG) proteins, helicases, and chromodomain proteins. There are five classes of histones, H1, H2A, H2B, H3, and H4, all of which are highly basic, low molecular weight proteins. The fundamental unit of chromatin, the nucleosome, consists of 200 base pairs of DNA associated with two copies each of H2A, H2B, H3, and H4. H1 links adjacent nucleosomes. HMG proteins are low molecular weight, non-histone proteins that may play a role in unwinding DNA and stabilizing single-stranded DNA. Helicases, which are DNA-dependent ATPases, unwind DNA, allowing access for transcription factors. Chromodomain proteins play a key role in the formation of highly-compacted, transcriptionally silent heterochromatin.

Many neoplastic disorders in humans can be attributed to inappropriate gene expression. Malignant cell growth may result from either excessive expression of tumor promoting genes or insufficient expression of tumor suppressor genes. (Cleary, M. L. (1992) Cancer Surv. 15:89-104.) Chromosomal translocations may also produce chimeric loci which fuse the coding sequence of one gene with the regulatory regions of a second unrelated gene. Such an arrangement often results in inappropriate gene transcription. The Wilms tumor suppressor gene product, WT1, is a protein containing a DNA-binding domain consisting of four zinc fingers and a proline-glutamine rich region capable of regulating transcription. (ExPASy PROSTIE document PR00049.) Deletions of the WT1 gene, or point mutations which destroy the DNA-binding activity of the protein are associated with development of the pediatric nephroblastoma, Wilms tumor, and Denys-Drash syndrome. (Rauscher, F. J. (1993) FASEB J. 7:896-903.)

Certain proteins enriched in glutamine are associated with various neurological disorders including spinocerebellar ataxia, bipolar effective disorder, schizophrenia, and autism. (Margolis, R. L. et al. (1997) Human Genetics 100:114-122.) These proteins contain regions with as many as 15 or more consecutive glutamine residues and may function as transcription factors with a potential role in regulation of neurodevelopment or neuroplasticity.

The immune system responds to infection or trauma by activating a cascade of events that coordinate the progressive selection, amplification, and mobilization of cellular defense mechanisms. A complex and balanced program of gene activation and repression is involved in this process. Hyperactivity of the immune system as a result of improper or insufficient regulation of gene expression may result in considerable tissue or organ damage. This damage is well documented in immunological responses associated with arthritis, allergens, heart attack, stroke, and infections. (Harrison's Principles of Internal Medicine, 13/e, McGraw Hill, Inc. and Teton Data Systems Software, 1996.) In particular, a zinc finger protein termed Staf50 (for Stimulated trans-acting factor of 50 kDa) is a transcriptional regulator and is induced in various cell lines by interferon-I and -II. Staf50 appears to mediate the antiviral activity of interferon by down-regulating the viral transcription directed by the long terminal repeat promoter region of human immunodeficiency virus type-1 in transfected cells (Tissot, C. (1995) J. Biol. Chem. 270:14891-14898).

The generation of multicellular organisms is based on the induction and coordination of cell differentiation at the appropriate stages of development. Differential gene expression confers the distinct identities of cells and tissues throughout the body. Failure to regulate gene expression during development could result in developmental disorders.

The discovery of new transcription factors and the polynucleotides encoding them satisfies a need in the art by providing new compositions which are useful in the diagnosis, prevention, and treatment of cell proliferative, autoimmune/inflanimatory, neurological, and developmental disorders, and in the assessment of the effects of exogenous compounds on the expression of nucleic acid and amino acid sequences of transcription factors.

### SUMMARY OF TEE INVENTION

The invention features purified polypeptides, transcription factors, referred to collectively as “TRFX” and individually as “TRFX-1,” “TRFX-2,” “TRFX-3,” “TRFX-4,” “TRFX-5,” “TRFX-6,” “TRFX-7,” “TRFX-8,” “TRFX-9,” “TRFX-10,” “TRFX-11,” “TRFX-12,” “TRFX-13,” “TRFX-14,” “TRFX-15,” “TRFX-16,” “TRFX-17,” “TRFX-18,” “TRFX-19,” “TRFX-20,” “TRFX-21,” “TRFX-22,” “TRFX-23,” “TRFX-24,” “TRFX-25,” “TRFX-26,” “TRFX-27,” “TRFX-28,” “TRFX-29,” “TRFX-30,” “TRFX-31,” “TRFX-32,” “TRFX-33,” “TRFX-34,” “TRFX-35,” “TRFX-36,” “TRFX-37,” “TRFX-38,” “TRFX-39,” “TRFX-40,” “TRFX-41,” “TRFX-42,” “TRFX-43,” “TRFX-44,” “TRFX-45,” “TRFX-46,” “TRFX-47,” “TRFX-48,” “TRFX-49,” “TRFX-50,” “TRFX-51,” “TRFX-52,” “TRFX-53,” “TRFX-54,” “TRFX-55,” “TRFX-56,” “TRFX-57,” “TRFX-58,” “TRFX-59,” “TRFX-60,” “TRFX-61,” “TRFX-62,” “TRFX-63,” “TRFX-64,” “TRFX-65,” “TRFX-66,” “TRFX-67,” “TRFX-68,” “TRFX-69,” “TRFX-70,” “TRFX-71,” “TRFX-72,” “TRFX-73,” “TRFX-74,” “TRFX-75,” “TRFX-76,” “TRFX-77,” “TRFX-78,” “TRFX-79,” “TRFX-80,” “TRFX-81,” “TRFX-82,” “TRFX-83,” “TRFX-84,” “TRFX-85,” “TRFX-86,” “TRFX-87,” “TRFX-88,” “TRFX-89,” “TRFX-90,” “TRFX-91,” “TRFX-92,” “TRFX-93,” “TRFX-94,” “TRFX-95,” “TRFX-96,” “TRFX-97,” “TRFX-98,” “TRFX-99,” “TRFX-100,” “TRFX-101,” “TRFX-102,” “TRFX-103,” “TRFX-104,” “TRFX-105,” “TRFX-106,” and “TRFX-107.” In one aspect, the invention provides an isolated polypeptide comprising an amino acid sequence selected from the group consisting of a) an amino acid sequence selected from the group consisting of SEQ ID NO:1-107, b) a naturally occurring amino acid sequence having at least 90% sequence identity to an amino acid sequence selected from the group consisting of SEQ ID NO:1-107, c) a biologically active fragment of an amino acid sequence selected from the group consisting of SEQ ID NO:1-107, and d) an immunogenic fragment of an amino acid sequence selected from the group consisting of SEQ ID NO:1-107. In one alternative, the invention provides an isolated polypeptide comprising the amino acid sequence of SEQ ID NO:1-107.

The invention further provides an isolated polynucleotide encoding a polypeptide comprising an amino acid sequence selected from the group consisting of a) an amino acid sequence selected from the group consisting of SEQ ID NO:1-107, b) a naturally occurring amino acid sequence having at least 90% sequence identity to an amino acid sequence selected from the group consisting of SEQ ID NO:1-107, c) a biologically active fragment of an amino acid sequence selected from the group consisting of SEQ ID NO:1-107, and d) an immunogenic fragment of an amino acid sequence selected from the group consisting of SEQ ID NO:1-107. In one alternative, the polynucleotide encodes a polypeptide selected from the group consisting of SEQ ID NO:1-107. In another alternative, the polynucleotide is selected from the group consisting of SEQ ID NO:108-214.

Additionally, the invention provides a recombinant polynucleotide comprising a promoter sequence operably linked to a polynucleotide encoding a polypeptide comprising an amino acid sequence selected from the group consisting of a) an amino acid sequence selected from the group consisting of SEQ ID NO:1-107, b) a naturally occurring amino acid sequence having at least 90% sequence identity to an amino acid sequence selected from the group consisting of SEQ ID NO:1-107, c) a biologically active fragment of an amino acid sequence selected from the group consisting of SEQ ID NO:1-107, and d) an immunogenic fragment of an amino acid sequence selected from the group consisting of SEQ ID NO:1-107. In one alternative, the invention provides a cell transformed with the recombinant polynucleotide. In another alternative, the invention provides a transgenic organism comprising the recombinant polynucleotide.

The invention also provides a method for producing a polypeptide comprising an amino acid sequence selected from the group consisting of a) an amino acid sequence selected from the group consisting of SEQ ID NO:1-107, b) a naturally occurring amino acid sequence having at least 90% sequence identity to an amino acid sequence selected from the group consisting of SEQ ID NO:1-107, c) a biologically active fragment of an amino acid sequence selected from the group consisting of SEQ ID NO:1-107, and d) an immunogenic fragment of an amino acid sequence selected from the group consisting of SEQ ID NO:1-107. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Additionally, the invention provides an isolated antibody which specifically binds to a polypeptide comprising an amino acid sequence selected from the group consisting of a) an amino acid sequence selected from the group consisting of SEQ ID NO:1-107, b) a naturally occurring amino acid sequence having at least 90% sequence identity to an amino acid sequence selected from the group consisting of SEQ ID NO:1-107, c) a biologically active fragment of an amino acid sequence selected from the group consisting of SEQ ID NO:1-107, and d) an immunogenic fragment of an amino acid sequence selected from the group consisting of SEQ ID NO:1-107.

The invention further provides an isolated polynucleotide comprising a polynucleotide sequence selected from the group consisting of a) a polynucleotide sequence selected from the group consisting of SEQ ID NO:108-214, b) a naturally occurring polynucleotide sequence having at least 90% sequence identity to a polynucleotide sequence selected from the group consisting of SEQ ID NO:108-214, c) a polynucleotide sequence complementary to a), d) a polynucleotide sequence complementary to b), and e) an RNA equivalent of a)-d). In one alternative, the polynucleotide comprises at least 60 contiguous nucleotides.

Additionally, the invention provides a method for detecting a target polynucleotide in a sample, said target polynucleotide having a sequence of a polynucleotide comprising a polynucleotide sequence selected from the group consisting of a) a polynucleotide sequence selected from the group consisting of SEQ ID NO:108-214, b) a naturally occurring polynucleotide sequence having at least 90% sequence identity to a polynucleotide sequence selected from the group consisting of SEQ ID NO:108-214, c) a polynucleotide sequence complementary to a), d) a polynucleotide sequence complementary to b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex, and optionally, if present, the amount thereof. In one alternative, the probe comprises at least 60 contiguous nucleotides.

The invention further provides a method for detecting a target polynucleotide in a sample, said target polynucleotide having a sequence of a polynucleotide comprising a polynucleotide sequence selected from the group consisting of a) a polynucleotide sequence selected from the group consisting of SEQ ID NO:108-214, b) a naturally occurring polynucleotide sequence having at least 90% sequence identity to a polynucleotide sequence selected from the group consisting of SEQ ID NO:108-214, c) a polynucleotide sequence complementary to a), d) a polynucleotide sequence complementary to b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof, and, optionally, if present, the amount thereof.

The invention further provides a composition comprising an effective amount of a polypeptide comprising an amino acid sequence selected from the group consisting of a) an amino acid sequence selected from the group consisting of SEQ ID NO:1-107, b) a natually occurring amino acid sequence having at least 90% sequence identity to an amino acid sequence selected from the group consisting of SEQ ID NO:1-107, c) a biologically active fragment of an amino acid sequence selected from the group consisting of SEQ ID NO:1-107, and d) an immunogenic fragment of an amino acid sequence selected from the group consisting of SEQ ID NO:1-107, and a pharmaceutically acceptable excipient. In one embodiment, the composition comprises an amino acid sequence selected from the group consisting of SEQ ID NO:1-107. The invention additionally provides a method of treating a disease or condition associated with decreased expression of functional TRFX, comprising administering to a patient in need of such treatment the composition.

The invention also provides a method for screening a compound for effectiveness as an agonist of a polypeptide comprising an amino acid sequence selected from the group consisting of a) an amino acid sequence selected from the group consisting of SEQ ID NO:1-107, b) a naturally occurring amino acid sequence having at least 90% sequence identity to an amino acid sequence selected from the group consisting of SEQ ID NO:1-107, c) a biologically active fragment of an amino acid sequence selected from the group consisting of SEQ ID NO:1-107, and d) an immunogenic fragment of an amino acid sequence selected from the group consisting of SEQ ID NO:1-107. The method comprises a) exposing a sample comprising the polypeptide to a compound, and b) detecting agonist activity in the sample. In one alternative, the invention provides a composition comprising an agonist compound identified by the method and a pharmaceutically acceptable excipient. In another alternative, the invention provides a method of treating a disease or condition associated with decreased expression of functional TRFX, comprising administering to a patient in need of such treatment the composition.

Additionally, the invention provides a method for screening a compound for effectiveness as an antagonist of a polypeptide comprising an amino acid sequence selected from the group consisting of a) an amino acid sequence selected from the group consisting of SEQ ID NO:1-107, b) a naturally occurring amino acid sequence having at least 90% sequence identity to an amino acid sequence selected from the group consisting of SEQ ID NO:1-107, c) a biologically active fragment of an amino acid sequence selected from the group consisting of SEQ ID NO:1-107, and d) an immunogenic fragment of an amino acid sequence selected from the group consisting of SEQ ID NO:1-107. The method comprises a) exposing a sample comprising the polypeptide to a compound, and b) detecting antagonist activity in the sample. In one alternative, the invention provides a composition comprising an antagonist compound identified by the method and a pharmaceutically acceptable excipient. In another alternative, the invention provides a method of treating a disease or condition associated with overexpression of functional TRFX, comprising adminstering to a patient in need of such treatment the composition.

The invention further provides a method of screening for a compound that specifically binds to a polypeptide comprising an amino acid sequence selected from the group consisting of a) an amino acid sequence selected from the group consisting of SEQ ID NO:1-107, b) a naturally occurring amino acid sequence having at least 90% sequence identity to an amino acid sequence selected from the group consisting of SEQ ID NO:1-107, c) a biologically active fragment of an amino acid sequence selected from the group consisting of SEQ ID NO:1-107, and d) an immunogenic fragment of an amino acid sequence selected from the group consisting of SEQ ID NO:1-107. The method comprises a) combining the polypeptide with at least one test compound under suitable conditions, and b) detecting binding of the polypeptide to the test compound, thereby identifying a compound that specifically binds to the polypeptide.

The invention further provides a method of screening for a compound that modulates the activity of a polypeptide comprising an amino acid sequence selected from the group consisting of a) an amino acid sequence selected from the group consisting of SEQ ID NO:1-107, b) a naturally occurring amino acid sequence having at least 90% sequence identity to an amino acid sequence selected from the group consisting of SEQ ID NO:1-107, c) a biologically active fragment of an amino acid sequence selected from the group consisting of SEQ ID NO:1-107, and d) an immunogenic fragment of an amino acid sequence selected from the group consisting of SEQ ID NO:1-107. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

The invention further provides a method for screening a compound for effectiveness in altering expression of a target polynucleotide, wherein said target polynucleotide comprises a sequence selected from the group consisting of SEQ ID NO:108-214, the method comprising a) exposing a sample comprising the target polynucleotide to a compound, and b) detecting altered expression of the target polynucleotide.

The invention further provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide comprising a polynucleotide sequence selected from the group consisting of i) a polynucleotide sequence selected from the group consisting of SEQ ID NO:108-214, ii) a naturally occurring polynucleotide sequence having at least 90% sequence identity to a polynucleotide sequence selected from the group consisting of SEQ ID NO:108-214, iii) a polynucleotide sequence complementary to i), iv) a polynucleotide sequence complementary to ii), and v) an RNA equivalent of i)-iv). Hybridization occurs under conditions whereby a specific hybridization complex is formed between said probe and a target polynucleotide in the biological sample, said target polynucleotide comprising a polynucleotide sequence selected from the group consisting of i) a polynucleotide sequence selected from the group consisting of SEQ ID NO:108-214, ii) a naturally occurring polynucleotide sequence having at least 90% sequence identity to a polynucleotide sequence selected from the group consisting of SEQ ID NO:108-214, iii) a polynucleotide sequence complementary to i), iv) a polynucleotide sequence complementary to ii), and v) an RNA equivalent of i)-iv). Alternatively, the target polynucleotide comprises a fragment of a polynucleotide sequence selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 shows polypeptide and nucleotide sequence identification numbers (SEQ ID NOs), clone identification numbers (clone IDs), cDNA libraries, and cDNA fragments used to assemble full-length sequences encoding TRFX.

Table 2 shows features of each polypeptide sequence, including potential motifs, homologous sequences, and methods, algorithms, and searchable databases used for analysis of TRFX.

Table 3 shows the tissue-specific expression patterns of each nucleic acid sequence as determined by northern analysis; diseases, disorders, or conditions associated with these tissues; and the vector into which each cDNA was cloned.

Table 4 describes the tissues used to construct the cDNA libraries from which cDNA clones encoding TRFX were isolated.

Table 5 shows the tools, programs, and algorithms used to analyze the polynucleotides and polypeptides of the invention, along with applicable descriptions, references, and threshold parameters.

### DESCREPTION OF TIHE INVENITON

Before the present proteins, nucleotide sequences, and methods are described, it is understood that this invention is not limited to the particular machines, materials and methods described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“ThFx” refers to the amino acid sequences of substantially purified TRFX obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of TRFX. Agonists may include proteins, nucleic acids, carbohydrates, small molecules, or any other compound or composition which modulates the activity of TRFX either by directly interacting with TRFX or by acting on components of the biological pathway in which TRFX participates.

An “allelic variant” is an alternative form of the gene encoding TRFX. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding TRFX include those sequences with deletions, insertions, or substitutions of different nucleotides, resulting in a polypeptide the same as TRFX or a polypeptide with at least one functional characteristic of TRFX. Included within this definition are polymorphisms which may or may not be readily detectable using a particular oligonucleotide probe of the polynucleotide encoding TRFX, and improper or unexpected hybridization to allelic variants, with a locus other than the normal chromosomal locus for the polynucleotide sequence encoding TRFX. The encoded protein may also be “altered,” and may contain deletions, insertions, or substitutions of amino acid residues which produce a silent change and result in a functionally equivalent TRPX. Deliberate amino acid substitutions may be made on the basis of similarity in polarity, charge, solubility, hydrophobicity, hydrophilicity, and/or the amphipathic nature of the residues, as long as the biological or immunological activity of TRFX is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” refer to an oligopeptide, peptide, polypeptide, or protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid sequence. Amplification is generally carried out using polymerase chain reaction (PCR) technologies well known in the art.

The term “antagonist” refers to a molecule which inhibits or attenuates the biological activity of TRFX. Antagonists may include proteins such as antibodies, nucleic acids, carbohydrates, small molecules, or any other compound or composition which modulates the activity of TRFX either by directly interacting with TRFX or by acting on components of the biological pathway in which TRFX participates.

The term “antibody” refers to intact immunoglobulin molecules as well as to fragments thereof, such as Fab, F(ab′), and Fv fragments, which are capable of binding an epitopic determinant. Antibodies that bind TRFX polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or threedimensional structures on the protein). An antigenic determinant may compete with the intact antigen (i.e., the immunogen used to elicit the immune response) for binding to an antibody.

The term “antisense” refers to any composition capable of base-pairing with the “sense” (coding) strand of a specific nucleic acid sequence. Antisense compositions may include DNA; RNA; peptide nucleic acid LENA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic TRFX, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide sequence” and a “composition comprising a given amino acid sequence” refer broadly to any composition containing the given polynucleotide or amino acid sequence. The composition may comprise a dry formulation or an aqueous solution. Compositions comprising polynucleotide sequences encoding TRFX or fragments of TRFX may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide sequence can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

A “fragment” is a unique portion of TRFX or the polynucleotide encoding TRFX which is identical in sequence to but shorter in length than the parent sequence. A fragment may comprise up to the entire length of the defined sequence, minus one nucleotide/amino acid residue. For example, a fragment may comprise from 5 to 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50% of a polypeptide) as shown in a certain defined sequence. Clearly these lengths are exemplary, and any length that is supported by the specification, including the Sequence Listing, tables, and figures, may be encompassed by the present embodiments.

A fragment of SEQ ID NO:108-214 comprises a region of unique polynucleotide sequence that specifically identifies SEQ ID NO:108-214, for example, as distinct from any other sequence in the genome from which the fragment was obtained. A fragment of SEQ ID NO:108-214 is useful, for example, in hybridization and amplification technologies and in analogous methods that distinguish SEQ ID NO:108-214 from related polynucleotide sequences. The precise length of a fragment of SEQ ID NO:108-214 and the region of SEQ ID NO:108-214 to which the fragment corresponds are routinely determinable by one of ordinary skill in the art based on the intended purpose for the fragment.

A fragment of SEQ ID NO:1-107 is encoded by a fragment of SEQ ID NO:108-214. A fragment of SEQ ID NO:1-107 comprises a region of unique amino acid sequence that specifically identifies SEQ ID NO:1-107. For example, a fragment of SEQ ID NO:1-107 is useful as an immunogenic peptide for the development of antibodies that specifically recognize SEQ ID NO:1-107. The precise length of a fragment of SEQ ID NO:1-107 and the region of SEQ ID NO:1-107 to which the fragment corresponds are routinely determinable by one of ordinary skill in the art based on the intended purpose for the fragment.

A “full-length” polynucleotide sequence is one containing at least a translation initiation codon (e.g., methionine) followed by an open reading frame and a translation termination codon. A “full-length” polynucleotide sequence encodes a “full-length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithim Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequences.

Alternatively, a suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST2 Sequences” can be accessed and used interactively at http://www.ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:

Matrix: BLOSUM62

Reward for match: 1

Penalty for mismatch: −2

Open Gap: 5 and Extension Gap: 2 penalties

Gap×drop-off 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:

Matrix: BLOSUM62

Open Gap: 11 and Extension Gap: 1 penalties

Gap×drop-off: 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACS) are linear microchromosomes which may contain DNA sequences of about 6 kb to 10 Mb in size, and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al., 1989, Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acid sequences by virtue of the formation of hydrogen bonds between complementary bases. A hybridization complex may be formed in solution (e.g., C₀t or R₀t analysis) or formed between one nucleic acid sequence present in solution and another nucleic acid sequence immobilized on a solid support (e.g., paper, membranes, filters, chips, pins or glass slides, or any other appropriate substrate to which cells or their nucleic acids have been fixed).

The words “insertion” and “addition” refer to changes in an amino acid or nucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of TRFX which is capable of eliciting an immune response when introduced into a living organism, for example, a mammal. The term “immunogenic fragment” also includes any polypeptide or oligopeptide fragment of TRFX which is useful in any of the antibody production methods disclosed herein or known in the art.

The term “microarray” refers to an arrangement of a plurality of polynucleotides, polypeptides, or other chemical compounds on a substrate.

The terms “element” and “array element” refer to a polynucleotide, polypeptide, or other chemical compound having a unique and defined position on a microarray.

The term “modulate” refers to a change in the activity of TRFX. For example, modulation may cause an increase or a decrease in protein activity, binding characteristics, or any other biological, functional, or immunological properties of TRFX.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an TRFX may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of TRFX.

“Probe” refers to nucleic acid sequences encoding TRFX, their complements, or fragments thereof, which are used to detect identical, allelic or related nucleic acid sequences. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid sequence, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989) Molecular Cloning: A Laboratory Manual, 2^{nd} ed., vol. 1-3, Cold Spring Harbor Press, Plainview N.Y.; Ausubel, F. M. et al. (1987) Current Protocols in Molecular Biology. Greene Publ. Assoc. & Wiley-Intersciences, New York N.Y.; “ins, M. et al. (1990) PCR Protocols, A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRS). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA sequence, is composed of the same linear sequence of nucleotides as the reference DNA sequence with the exception that all occurrences of the nitrogenous base thyinine are replaced with uracil, and the sugar backbone is composed of ribose instead of deoxyribose.

The term “sample” is used in its broadest sense. A sample suspected of containing nucleic acids encoding TRFX, or fragments thereof, or TRFX itself, may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least 60% free, preferably at least 75% free, and most preferably at least 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock lipofection, and particle bombardment. The term “transformed” cells includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants, and animals. The isolated DNA of the present invention can be introduced into the host by methods known in the art, for example infection, transfection, transformation or transconjugation. Techniques for transferring the DNA of the present invention into such organisms are widely known and provided in references such as Sambrook, J. et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 95% or at least 98% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternative splicing of exons during mRNA processing. The corresponding polypeptide may possess additional functional domains or lack domains that are present in the reference molecule. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 95%, or at least 98% or greater sequence identity over a certain defined length of one of the polypeptides.

The Invention

The invention is based on the discovery of new human transcription factors (TRFX), the polynucleotides encoding TRFX, and the use of these compositions for the diagnosis, treatrnent, or prevention of cell proliferative, autoimmune/inflanmnatory, neurological, and developmental disorders.

Table 1 lists the Incyte clones used to assemble full length nucleotide sequences encoding TRFX. Columns 1 and 2 show the sequence identification numbers (SEQ ID NOs) of the polypeptide and nucleotide sequences, respectively. Column 3 shows the clone IDs of the Incyte clones in which nucleic acids encoding each TRFX were identified, and column 4 shows the cDNA libraries from which these clones were isolated. Column 5 shows Incyte clones and their corresponding cDNA libraries. Clones for which cDNA libraries are not indicated were derived from pooled cDNA libraries. In some cases, GenBank sequence identifiers are also shown in column 5. The Incyte clones and GenBank cDNA sequences, where indicated, in column 5 were used to assemble the consensus nucleotide sequence of each TRFX and are useful as fragments in hybridization technologies.

The columns of Table 2 show various properties of each of the polypeptides of the invention: column 1 references the SEQ ID NO and Incyte clone ID of each polypeptide; column 2 shows the number of amino acid residues in each polypeptide; column 3 shows potential phosphorylation sites; column 4 shows potential glycosylation sites; column 5 shows the amino acid residues comprising signature sequences and motifs; column 6 shows homologous sequences as identified by BLAST analysis along with relevant citations, all of which are expressly incorporated by reference herein in their entirety; and column 7 shows analytical methods and in some cases, searchable databases to which the analytical methods were applied. The methods of column 7 were used to characterize each polypeptide through sequence homology and protein motifs.

The columns of Table 3 show the tissue-specificity and diseases, disorders, or conditions associated with nucleotide sequences encoding TRFX. The first column of Table 3 lists the nucleotide SEQ ID NOs and Incyte Clone IDs. Fragments of these polynucleotides are useful, for example, in hybridization or amplification technologies to identify SEQ ID NO:108-214 and to distinguish between SEQ ID NO:108-214 and related polynucleotide sequences. The polypeptides encoded by these fragments are useful, for example, as immunogenic peptides. Column 2 lists tissue categories which express TRFX as a fraction of total tissues expressing TRFX. Column 3 lists diseases, disorders, or conditions associated with those tissues expressing TRFX as a fraction of total tissues expressing TRFX. Column 4 lists the vectors used to subclone each cDNA library.

The columns of Table 4 show descriptions of the tissues used to construct the cDNA libraries from which cDNA clones encoding TRFX were isolated. Column 1 references the nucleotide SEQ ID NOs and Incyte Clone IDs, column 2 shows the cDNA libraries from which these clones were isolated, and column 3 shows the tissue origins and other descriptive information relevant to the cDNA libraries in column 2.

SEQ ID NO:111 maps to chromosome 6 within the interval from 89.4 to 96.1 centiMorgans.

SEQ ID NO:114 maps to chromosome 6 within the interval from 42.0 to 44.9 centiMorgans.

SEQ ID NO:117 maps to chromosome 13 within the interval from 95.9 to 112.7 centiMorgans.

SEQ ID NO:122 maps to chromosome 3 within the interval from 55.4 to 63.3 centiMorgans.

SEQ ID NO:123 maps to chromosome 7 within the interval from 149.6 to 159.0 centiMorgans.

SEQ ID NO:125 maps to chromosome 15 within the interval from 45.5 to 58.8 centiMorgans.

SEQ ID NO:130 maps to chromosome 1 within the interval from 152.2 to 156.1 centiMorgans.

SEQ ID NO:132 maps to chromosome 1 within the interval from 36.2 to 54.2 centiMorgans.

SEQ ID NO:133 maps to chromosome 19 within the interval from 41.7 to 49.4 centiMorgans.

SEQ ID NO:134 maps to chromosome 17 within the interval from 99.3 to 104.7 centiMorgans.

SEQ ID NO:136 maps to chromosome 16 within the interval from 119.2 centiMorgans to the q-terminus.

SEQ ID NO:138 maps to chromosome 19 within the interval from 60.9 to 61.4 centiMorgans.

SEQ ID NO:145 maps to chromosome 2 within the interval from 190.8 to 196.8 centiMorgans and to chromosome 10 within the interval from 68.7 to 72.5 centiMorgans.

SEQ ID NO:149 maps to chromosome 3 within the interval from the p terminus to 16.5 centiMorgans.

SEQ ID NO:152 maps to chromosome 19 within the interval from 35.5 to 49.4 centiMorgans and to chromosome 7 within the interval from 100.5 to 114.5 centiMorgans and to chromosome 7 within the intervals from 67.6 to 69.3 centiMorgans and 83.8 centiMorgans and the q-terminus.

SEQ ID NO:153 maps to chromosome 16 within the interval from 65.6 to 72.6 centiMorgans.

SEQ ID NO:156 maps to chromosome 20 within the interval from 65.5 to 79.0 centiMorgans.

SEQ ID NO:159 maps to chromosome 18 within the interval from 40.4 to 49.7 centiMorgans.

SEQ ID NO:168 maps to chromosome 23 within the interval from 112.8 to 139.4 centiMorgans.

SEQ ID NO:179 maps to chromosome 11 within the interval from 16.7 to 24.7 centiMorgans.

SEQ ID NO:180 maps to chromosome 16 within the interval from 33.3 to 42.7 centiMorgans

SEQ ID NO:184 maps to chromosome 2 within the interval from 190.5 to 196.8 centiMorgans and within the interval from the p terminus to 16.4 centiMorgans.

SEQ ID NO:185 maps to chromosome 9 within the interval from 20.4 to 27.8 centiMorgans and from the p terminus to 33.3 centiMorgans.

SEQ ID NO:196 maps to chromosome 1 within the interval from 57.2 to 57.5 centiMorgans.

SEQ ID NO:197 maps to chromosome 19 within the interval from 60.9 to 61.4 centiMorgans.

SEQ ID NO:199 maps to chromosome 13 within the interval from 77.1 to 86.9 centiMorgans and to chromosome 2 within the interval from 51.2 to 51.8 centiMorgans.

SEQ ID NO:201 maps to chromosome 22 within the interval from 22.2 to 40.2 centiMorgans.

SEQ ID NO:204 maps to chromosome 5 within the interval from 132.8 to 141.4 centiMorgans.

SEQ ID NO:208 maps to chromosome 13 within the interval from 37.3 to 45.8 centiMorgans and to chromosome 19 within the interval from 58.1 to 58.7 centiMorgans.

SEQ ID NO:212 maps to chromosome 19 within the interval from the p terminus to 35.5 centiMorgans and to chromosome 20 within the interval from 50.2 to 53.6.

SEQ ID NO:213 maps to chromosome 6 within the interval from the p terminus to 14.2 centiMorgans.

The invention also encompasses TRFX variants. A preferred TRFX variant is one which has at least about 80%, or alternatively at least about 90%, or even at least about 95% amino acid sequence identity to the TRFX amino acid sequence, and which contains at least one functional or structural characteristic of TRFX.

The invention also encompasses polynucleotides which encode TRFX. In a particular embodiment, the invention encompasses a polynucleotide sequence comprising a sequence selected from the group consisting of SEQ ID NO:108-214, which encodes TRFX. The polynucleotide sequences of SEQ ID NO:108-214, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses a variant of a polynucleotide sequence encoding TRFX. In particular, such a variant polynucleotide sequence will have at least about 70%, or alternatively at least about 85%, or even at least about 95% polynucleotide sequence identity to the polynucleotide sequence encoding TRFX. A particular aspect of the invention encompasses a variant of a polynucleotide sequence comprising a sequence selected from the group consisting of SEQ ID NO:108-214 which has at least about 70%, or alternatively at least about 85%, or even at least about 95% polynucleotide sequence identity to a nucleic acid sequence selected from the group consisting of SEQ ID NO:108-214. Any one of the polynucleotide variants described above can encode an amino acid sequence which contains at least one functional or structural characteristic of TRFX.

It will be appreciated by those skilled in the art that as a result of the degeneracy of the genetic code, a multitude of polynucleotide sequences encoding TRFX, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring TRFX, and all such variations are to be considered as being specifically disclosed.

Although nucleotide sequences which encode TRFX and its variants are generally capable of hybridizing to the nucleotide sequence of the naturally occurring TRFX under appropriately selected conditions of stringency, it may be advantageous to produce nucleotide sequences encoding TRFX or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding TRFX and its derivatives without altering the encoded amino acid sequences include the production of RNA transcripts having more desirable properties, such as a greater half-life, than transcripts produced from the naturally occurring sequence.

The invention also encompasses production of DNA sequences which encode TRFX and TRFX derivatives, or fragments thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding TRFX or any fragment thereof.

Also encompassed by the invention are polynucleotide sequences that are capable of hybridizing to the claimed polynucleotide sequences, and, in particular, to those shown in SEQ ID NO:108-214 and fragments thereof under various conditions of stringency. (See, e.g., Wahl, G. M. and S. L. Berger (1987) Methods Enzymol. 152:399407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems, Foster City Calif.), thermostable T7 polymerase (Amersham Pharmacia Biotech, Piscataway N.J.), or combinations of polymerases and proofreading exonucleases such as those found in the ELONGASE amplification system (Life Technologies, Gaithersburg Md.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Molecular Dynamics, Sunnyvale Calif.), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

The nucleic acid sequences encoding TRFX may be extended utilizing a partial-nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences in human and yeast artificial chromosome DNA. (See, e.g., Lagerstrom, M. et al. (1991) PCR Methods Applic. 1: 111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 Primer Analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full-length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or fragments thereof which encode TRFX may be cloned in recombinant DNA molecules that direct expression of TRFX, or fragments or functional equivalents thereof, in appropriate host cells. Due to the inherent degeneracy of the genetic code, other DNA sequences which encode substantially the same or a functionally equivalent amino acid sequence may be produced and used to express TRFX.

The nucleotide sequences of the present invention can be engineered using methods generally known in the art in order to alter TRFX-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, P. C. et al. (1999) Nat. Biotechnol. 17:259-264; and Crameri, A. et al. (1996) Nat. Biotechnol. 14:315-319) to alter or improve the biological properties of TRFX, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, sequences encoding TRFX may be synthesized, in whole or in part, using chemical methods well known in the art. (See, e.g., Caruthers, M. H. et al. (1980) Nucleic Acids Symp. Ser. 7:215-223; Horn, T. et al. (1980) Nucleic Acids Symp. Ser. 7:225-232.) Alternatively, TRFX itself or a fragment thereof may be synthesized using chemical methods. For example, peptide synthesis can be performed using various solution-phase or solid-phase techniques. (See, e.g., Creighton, T. (1984) Proteins, Structures and Molecular Properties, W H Freeman, New York N.Y., pp. 55-60; and Roberge, J. Y. et al. (1995) Science 269:202-204.) Automated synthesis may be achieved using the ABI 431A peptide synthesizer (Applied Biosystems). Additionally, the amino acid sequence of TRFX, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography. (See, e.g., Chiez, R. M. and F. Z. Regnier (1990) Methods Enzymol. 182:392-421.) The composition of the synthetic peptides may be confirmed by amino acid analysis or by sequencing. (See, e.g., Creighton, supra, pp. 28-53.)

In order to express a biologically active TRFX, the nucleotide sequences encoding TRFX or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding TRFX. Such elements may vary in their strength and specificity. Specific initiation signals may also be used to achieve more efficient translation of sequences encoding TRFX. Such signals include the ATG initiation codon and adjacent sequences, e.g. the Kozak sequence. In cases where sequences encoding TRFX and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20:125-162.)

Methods which are well known to those skilled in the art may be used to construct expression vectors containing sequences encoding TRFX and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16.)

A variety of expression vector/host systems may be utilized to contain and express sequences encoding TRFX. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, sulpra; Ausubel, supra; Van Heeke, G. and S. M. Schuster (1989) J. Biol. Chem. 264:5503-5509; Bitter, G. A. et al. (1987) Methods Enzymol. 153:516-544; Scorer, Calif. et al. (1994) Bio/Technology 12:181-184; Engelhard, E. K. et al. (1994) Proc. Nat. Acad. Sci. USA 91:3224-3227; Sandig, V. et al. (1996) Hum. Gene Ther. 7:1937-1945; Takamatsu, N. (1987) EMBO J. 6:307-311; Coruzzi, G. et al. (1984) EMBO J. 3:1671-1680; Broglie, R. et al. (1984) Science 224:838-843; Winter, J. et al. (1991) Results Probl. Cell Differ. 17:85-105; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (1997) Nat. Genet. 15:345-355.) Expression vectors derived from retroviruses, adenoviruses, or herpes or vaccinia viruses, or from various bacterial plasmids, may be used for delivery of nucleotide sequences to the targeted organ, tissue, or cell population. (See, e.g., Di Nicola, M. et al. (1998) Cancer Gen. Ther. 5(6):350356; Yu, M. et al. (1993) Proc. Natl. Acad. Sci. USA 90(13):6340-6344; Buller, R. M. et al. (1985) Nature 317(6040):813-815; McGregor, D. P. et al. (1994) Mol. Immunol. 31(3):219-226; and Verma, I. M. and N. Somia (1997) Nature 389:239-242.) The invention is not limited by the host cell employed.

In bacterial systems, a number of cloning and expression vectors may be selected depending upon the use intended for polynucleotide sequences encoding TRFX. For example, routine cloning, subcloning, and propagation of polynucleotide sequences encoding TRFX can be achieved using a multifunctional E. coli vector such as PBLUESCRIPT (Stratagene, La Jolla Calif.) or PSPORT1 plasmid (Life Technologies). Ligation of sequences encoding TRFX into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of TRFX are needed, e.g. for the production of antibodies, vectors which direct high level expression of TRFX may be used. For example, vectors containing the strong, inducible T5 or T7 bacteriophage promoter may be used.

Yeast expression systems may be used for production of TRFX. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign sequences into the host genome for stable propagation. (See, e.g., Ausubel, 1995, surra; Bitter, supra; and Scorer, supra.)

Plant systems may also be used for expression of TRFX. Transcription of sequences encoding TRFX may be driven viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used. (See, e.g., Conizzi, supra; Broglie, supra; and Winter, supra.) These constructs can be introduced into plant ceus by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, sequences encoding TRFX may be ligated into an adenovirus transcription/translation complex consisting of the late promoter and tripartite leader sequence. Insertion in a non-essential E1 or E3 region of the viral genome may be used to obtain infective virus which expresses TRFX in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes. (See, e.g., Harrington, J. J. et al. (1997) Nat. Genet. 15:345-355.)

For long term production of recombinant proteins in mammalian systems, stable expression of TRRX in cell lines is preferred. For example, sequences encoding TRFX can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively. (See, e.g., Wigler, M. et al. (1977) Cell 11:223-232; Lowy, I. et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulflron and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14.) Additional selectable genes have been described, e.g., trpb and hisD, which alter cellular requirements for metabolites. (See, e.g., Hartan, S. C. and R. C. Mulligan (1988) Proc. Natl. Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding TRFX is inserted within a marker gene sequence, transformed cells containing sequences encoding TRFX can be identified by the absence of marker gene function. Alternatively, a marker gene can be placed in tandem with a sequence encoding TRFX under the control of a single promoter. Expression of the marker gene in response to induction or selection usually indicates expression of the tandem gene as well.

In general, host cells that contain the nucleic acid sequence encoding TRFX and that express TRFX may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of TRFX using either specific polyclonal or monoclonal antibodies are known in the art. Examples of such techniques include enzyme-linked irmnunosorbent assays (ELISAs), radioimmunoassays (RIAs), and fluorescence activated cell sorting (FACS). A two-site, monoclonal-based immunoassay utilizing monoclonal antibodies reactive to two non-interferi itopes on TRFX is preferred, but a competitive binding assay may be employed. These and other assays are well known in the art. (See, e.g., Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding TRFX include oligolabeling, nick translation, end-labeling, or PCR amplification using a labeled nucleotide. Alternatively, the sequences encoding TRFX, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Pharmacia Biotech, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with nucleotide sequences encoding TRFX may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode TRFX may be designed to contain signal sequences which direct secretion of TRFX through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant nucleic acid sequences encoding TRFX may be ligated to a heterologous sequence resulting in translation of a fusion protein in any of the aforementioned host systems. For example, a chimeric TRFX protein containing a heterologous moiety that can be recognized by a commercially available antibody may facilitate the screening of peptide libraries for inhibitors of TRFX activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the TRFX encoding sequence and the heterologous protein sequence, so that TRFX may be cleaved away from the heterologous moiety following purification. Methods for fusion protein expression and purification are discussed in Ausubel (1995, supra, ch. 10). A variety of commercially available kits may also be used to facilitate expression and purification of fusion proteins.

In a further embodiment of the invention, synthesis of radiolabeled TRFX may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

TRFX of the present invention or fragments thereof may be used to screen for compounds that specifically bind to TRFX. At least one and up to a plurality of test compounds may be screened for specific binding to TRFX. Examples of test compounds include antibodies, oligonucleotides, proteins (e.g., receptors), or small molecules.

In one embodiment, the compound thus identified is closely related to the natural ligand of TRFX, e.g., a ligand or fragment thereof, a natural substrate, a structural or functional mimetic, or a natural binding partner. (See, e.g., Coligan, J. E. et al. (1991) Current Protocols in Immunology 1(2): Chapter 5.) Similarly, the compound can be closely related to the natural receptor to which TRFX binds, or to at least a fragment of the receptor, e.g., the ligand binding site. In either case, the compound can be rationally designed using known techniques. In one embodiment, screening for these compounds involves producing appropriate cells which express TRFX, either as a secreted protein or on the cell membrane. Preferred cells include cells from mammals, yeast, Drosophila, or E. coli. Cells expressing TRFX or cell membrane fractions which contain TRFX are then contacted with a test compound and binding, stimulation, or inhibition of activity of either TRFX or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with TRFX, either in solution or affixed to a solid support, and detecting the binding of TRFX to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

TRFX of the present invention or fragments thereof may be used to screen for compounds that modulate the activity of TRFX. Such compounds may include agonists, antagonists, or partial or inverse agonists. In one embodiment, an assay is performed under conditions permissive for TRFX activity, wherein TRFX is combined with at least one test compound, and the activity of TRFX in the presence of a test compound is compared with the activity of TRFX in the absence of the test compound. A change in the activity of TRFX in the presence of the test compound is indicative of a compound that modulates the activity of TRFX. Alternatively, a test compound is combined with an in vitro or cell-free system comprising TRFX under conditions suitable for TRFX activity, and the assay is performed. In either of these assays, a test compound which modulates the activity of TRFX may do so indirectly and need not come in direct contact with the test compound. At least one and up to a plurality of test compounds may be screened.

In another embodiment, polynucleotides encoding TRFX or their mammalian homologs may be “knocked out” in an animal model system using homologous recombination in embryonic stem (ES) cells. Such techniques are well known in the art and are useful for the generation of animal models of human disease. (See, e.g., U.S. Pat. No. 5,175,383 and U.S. Pat. No. 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:43234330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding TRFX may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding TRFX can also be used to create “knockin” humanized animals (pigs) or transgenic animals (mice or rats) to model human disease. With knockin technology, a region of a polynucleotide encoding TRPX is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress TRFX, e.g., by secreting TRFX in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of TRFX and transcription factors. In addition, the expression of TRFX is closely associated with reproductive, nervous, and hematopoeitic/immune tissues. Therefore, TRFX appears to play a role in cell proliferative, autoimmune/inflammatory, neurological, and developmental disorders. In the treatment of disorders associated with increased TRFX expression or activity, it is desirable to decrease the expression or activity of TRFX. In the treatment of disorders associated with decreased TRFX expression or activity, it is desirable to increase the expression or activity of TRFX.

Therefore, in one embodiment, TRFX or a fragment or derivative thereof may be administered to a subject to treat or prevent a disorder associated with decreased expression or activity of TRFX. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjogren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; and a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss.

In another embodiment, a vector capable of expressing TRFX or a fragment or derivative thereof may be administered to a subject to treat or prevent a disorder associated with decreased expression or activity of TRFX including, but not limited to, those described above.

In a further embodiment, a composition comprising a substantially purified TRFX in conjunction with a suitable pharmaceutical carrier may be administered to a subject to treat or prevent a disorder associated with decreased expression or activity of TRFX including, but not limited to, those provided above.

In still another embodiment, an agonist which modulates the activity of TRFX may be administered to a subject to treat or prevent a disorder associated with decreased expression or activity of TRFX including, but not limited to, those listed above.

In a further embodiment, an antagonist of TRFX may be administered to a subject to treat or prevent a disorder associated with increased expression or activity of TRFX. Examples of such disorders include, but are not limited to, those cell proliferative, autoimmune/inflammatory, neurological, and developmental disorders described above. In one aspect, an antibody which specifically binds TRFX may be used directly as an antagonist or indirectly as a targeting or delivery mechanism for bringing a pharmaceutical agent to cells or tissues which express TRFX.

In an additional embodiment, a vector expressing the complement of the polynucleotide encoding TRFX may be administered to a subject to treat or prevent a disorder associated with increased expression or activity of TRFX including, but not limited to, those described above.

In other embodiments, any of the proteins, antagonists, antibodies, agonists, complementary sequences, or vectors of the invention may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of TRFX may be produced using methods which are generally known in the art. In particular, purified TRFX may be used to produce antibodies or to screen libraries of pharmaceutical agents to identify those which specifically bind TRFX. Antibodies to TRFX may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use.

For the production of antibodies, various hosts including goats, rabbits, rats, mice, humans, and others may be immunized by injection with TRFX or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to TRFX have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of TRFX amino acids may be fused with those of another protein, such as KLH, and antibodies to the chimeric molecule may be produced.

Monoclonal antibodies to TRX may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495497; Kozbor, D. et al. (1985) J. Immunol. Methods 81:3142; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452454.) Alternatively, techniques described for the production of single chain antibodies may be adapted, using methods known in the art, to produce TRFX-specific single chain antibodies. Antibodies with related specificity, but of distinct idiotypic composition, may be generated by chain shuffling from random combinatorial immunoglobulin libraries. (See, e.g., Burton, D. R. (1991) Proc. Natl. Acad. Sci. USA 88:10134-10137.)

Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for TRFX may also be generated. For example, such fragments include, but are not limited to, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between TRFX and its specific antibody. A two-site, monoclonal-based immunoassay utilizing monoclonal antibodies reactive to two non-interfering TRFX epitopes is generally used, but a competitive binding assay may also be employed (Pound, supra).

Various methods such as Scatchard analysis in conjunction with radioimmunoassay techniques may be used to assess the affinity of antibodies for TRFX. Affinity is expressed as an association constant, Kₐ, which is defined as the molar concentration of TRFX-antibody complex divided by the molar concentrations of free antigen and free antibody under equilibrium conditions. The Kₐ determined for a preparation of polyclonal antibodies, which are heterogeneous in their affinities for multiple TRFX epitopes, represents the average affinity, or avidity, of the antibodies for TRFX. The Kₐ determined for a preparation of monoclonal antibodies, which are monospecific for a particular TRFX epitope, represents a true measure of affinity. High-affinity antibody preparations with Kₐ ranging from about 10⁹ to 10¹² L/mole are preferred for use in immunoassays in which the TRFX-antibody complex must withstand rigorous manipulations. Low-affinity antibody preparations with Kₐ ranging from about 10⁶ to 10⁷ L/mole are preferred for use in immunopurification and similar procedures which ultimately require dissociation of TRFX, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of TRFX-antibody complexes. Procedures for evaluating antibody specificity, titer, and avidity, and guidelines for antibody quality and usage in various applications, are generally available. (See, e.g., Catty, supra, and Coligan et al., supra.)

In another embodiment of the invention, the polynucleotides encoding TRFX, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding TRFX. Such technology is well known in the art, and antisense oligonucleotides or larger fragments can be designed from various locations along the coding or control regions of sequences encoding TRFX. (See, e.g., Agrawal, S., ed. (1996) Antisense Therapeutics, Humana Press Inc., Totawa N.J.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein. (See, e.g., Slater, J. E. et al. (1998) J. Allergy Clin. Immunol. 102(3):469-475; and Scanlon, K. J. et al. (1995) 9(13):1288-1296.) Antisense sequences can also be introduced intracellularly through the use of viral vectors, such as retrovirus and adeno-associated virus vectors. (See, e.g., Miller, A. D. (1990) Blood 76:271; Ausubel, supra; Uckert, W. and W. Walther (1994) Pharmacol. Ther. 63(3):323-347.) Other gene delivery mechanisms include liposome-derived systems, artificial viral envelopes, and other systems known in the art. (See, e.g., Rossi, J. J. (1995) Br. Med. Bull. 51(1):217-225; Boado, R. J. et al. (1998) J. Pharm. Sci. 87(11):1308-1315; and Morris, M. C. et al. (1997) Nucleic Acids Res. 25(14):2730-2736.)

In another embodiment of the invention, polynucleotides encoding TRFX may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzanaalvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum Gene Therapy 6:667-703), thalassamias, familial hypercholesterolemia, and hemophilia resulting from Factor VIII or Factor IX deficiencies (Crystal, R. G. (1995) Science 270:404410; Verma, IM. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HN) (Baltimore, D. (1988) Nature 335:395-396; Poeschla, E. et al. (1996) Proc. Natl. Acad. Sci. USA. 93:11395-11399), hepatitis B or C virus (HBV, HCV); fungal parasites, such as Candida albicans and Paracoccidioides brasiliensis; and protozoan parasites such as Plasmodium falciparum and Tryvanosoma cruzi). In the case where a genetic deficiency in TRFX expression or regulation causes disease, the expression of TRFX from an appropriate population of transduced cells may alleviate the clinical manifestations caused by the genetic deficiency.

In a further embodiment of the invention, diseases or disorders caused by deficiencies in TRFX are treated by constructing mammalian expression vectors encoding TRFX and introducing these vectors by mechanical means into TRFX-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. Anderson (1993) Annu. Rev. Biochem. 62:191-217; Ivics, Z. (1997) Cell 91:501-510; Boulay, J-L. and H. Récipon (1998) Curr. Opin. Biotechnol. 9:445-450).

Expression vectors that may be effective for the expression of TRFX include, but are not limited to, the PCDNA 3.1, EPITAG, PRCCMV2, PREP, PVAX vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). TRFX may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding TRFX from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to TRFX expression are treated by constructing a retrovirus vector consisting of (i) the polynucleotide encoding TRFX under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are cornmercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M L. (1997) J. Virol. 71:4707-4716; Ranga, U. et al. (1998) Proc. Natl. Acad. Sci. USA 95:1201-1206; Su, L. (1997) Blood 89:2283-2290).

In the alternative, an adenovirus-based gene therapy delivery system is used to deliver polynucleotides encoding TRFX to cells which have one or more genetic abnormalities with respect to the expression of TRFX. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544; and Verma, L. M. and N. Somia (1997) Nature 18:389:239-242, both incorporated by reference herein.

In another alternative, a herpes-based, gene therapy delivery system is used to deliver polynucleotides encoding TRFX to target cells which have one or more genetic abnormalities with respect to the expression of TRFX. The use of herpes simplex virus (HSV)-based vectors may be especially valuable for introducing TRFX to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999) J. Virol. 73:519-532 and Xu, H. et al. (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another alternative, an alphavirus (positive, single-stranded RNA virus) vector is used to deliver polynucleotides encoding TRFX to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464469). During alphavirus RNA replication, a subgenomic RNA is generated that normally encodes the viral capsid proteins. This subgenornic RNA replicates to higher levels than the full-length genomic RNA, resulting in the overproduction of capsid proteins relative to the viral proteins with enzymatic activity (e.g., protease and polymerase). Similarly, inserting the coding sequence for TRFX into the alphavirus genome in place of the capsid-coding region results in the production of a large number of TRFX-coding RNAs and the synthesis of high levels of TRFX in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of TRFX into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, e.g., Gee, J. E. et al. (1994) in Huber, B. E. and B. I. Carr, Molecular and Immunologic Aproaches, Futura Publishing, Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding TRFX.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding TRFX. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endoniucleases.

An additional embodiment of the invention encompasses a method for screening for a compound which is effective in altering expression of a polynucleotide encoding TRFX. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased TRFX expression or activity, a compound which specifically inhibits expression of the polynucleotide encoding TRFX may be therapeutically useful, and in the treatment of disorders associated with decreased TRFX expression or activity, a compound which specifically promotes expression of the polynucleotide encoding TRFX may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding TRFX is exposed to at least one test compound thus obtained. The sample may comprise, for example, an intact or permeabilized cell, or an in vitro cell-free or reconstituted biochemical system. Alterations in the expression of a polynucleotide encoding TRFX are assayed by any method commonly known in the art. Typically, the expression of a specific nucleotide is detected by hybridization with a probe having a nucleotide sequence complementary to the sequence of the polynucleotide encoding TRFX. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomvces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nat. Biotechnol. 15:462-466.)

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of TRFX, antibodies to TRFX, and mimetics, agonists, antagonists, or inhibitors of TRFX.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising TRFX or fragments thereof. For example, liposome preparations containing a cell-impermeable macromolecule may promote cell fusion and intracellular delivery of the macromolecule. Alternatively, TRFX or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example TRFX or fragments thereof, antibodies of TRFX, and agonists, antagonists or inhibitors of TRFX, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekldy depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind TRFX may be used for the diagnosis of disorders characterized by expression of TRFX, or in assays to monitor patients being treated with TRFX or agonists, antagonists, or inhibitors of TRFX. Antibodies useful for diagnostic purposes may be prepared in the same manner as described above for therapeutics. Diagnostic assays for TRFX include methods which utilize the antibody and a label to detect TRFX in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring TRFX, including ELISAs, RIAs, and FACS, are known in the art and provide a basis for diagnosing altered or abnormal levels of TRFX expression. Normal or standard values for TRFX expression are established by combining body fluids or cell extracts taken from normal mammalian subjects, for example, human subjects, with antibody to TRFX under conditions suitable for complex formation. The amount of standard complex formation may be quantitated by various methods, such as photometric means. Quantities of TRFX expressed in subject, control, and disease samples from biopsied tissues are compared with the standard values. Deviation between standard and subject values establishes the parameters for diagnosing disease.

In another embodiment of the invention, the polynucleotides encoding TRFX may be used for diagnostic purposes. The polynucleotides which may be used include oligonucleotide sequences, complementary RNA and DNA molecules, and PNAs. The polynucleotides may be used to detect and quantify gene expression in biopsied tissues in which expression of TRFX may be correlated with disease. The diagnostic assay may be used to determine absence, presence, and excess expression of TRFX, and to monitor regulation of TRFX levels during therapeutic intervention.

In one aspect, hybridization with PCR probes which are capable of detecting polynucleotide sequences, including genomic sequences, encoding TRFX or closely related molecules may be used to identify nucleic acid sequences which encode TRFX. The specificity of the probe, whether it is made from a highly specific region, e.g., the 5′regulatory region, or from a less specific region, e.g., a conserved motif, and the stringency of the hybridization or amplification will determine whether the probe identifies only naturally occurring sequences encoding TRFX, allelic variants, or related sequences.

Probes may also be used for the detection of related sequences, and may have at least 50% sequence identity to any of the TRFX encoding sequences. The hybridization probes of the subject invention may be DNA or RNA and may be derived from the sequence of SEQ ID NO:108-214 or from genomic sequences including promoters, enhancers, and introns of the TRFX gene.

Means for producing specific hybridization probes for DNAs encoding TRFX include the cloning of polynucleotide sequences encoding TRFX or TRFX derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotide sequences encoding TRFX may be used for the diagnosis of disorders associated with expression of TRFX. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoinmmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scieroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; and a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss. The polynucleotide sequences encoding TRFX may be used in Southern or northern analysis, dot blot, or other membrane-based technologies; in PCR technologies; in dipstick, pin, and multiformat ELISA-like assays; and in microarrays utilizing fluids or tissues from patients to detect altered TRFX expression. Such qualitative or quantitative methods are well known in the art.

In a particular aspect, the nucleotide sequences encoding TRFX may be useful in assays that detect the presence of associated disorders, particularly those mentioned above. The nucleotide sequences encoding TRFX may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of nucleotide sequences encoding TRFX in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of TRFX, a normal or standard profile for expression is established. This may be accomplished by combining body fluids or cell extracts taken from normal subjects, either animal or human, with a sequence, or a fragment thereof, encoding TRFX, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Additional diagnostic uses for oligonucleotides designed from the sequences encoding TRFX may involve the use of PCR. These oligomers may be chemically synthesized, generated enzymatically, or produced in vitro. Oligomers will preferably contain a fragment of a polynucleotide encoding TRFX, or a fragment of a polynucleotide complementary to the polynucleotide encoding TRFX, and will be employed under optimized conditions for identification of a specific gene or condition. Oligomers may also be employed under less stringent conditions for detection or quantification of closely related DNA or RNA sequences.

In a particular aspect, oligonucleotide primers derived from the polynucleotide sequences encoding TRFX may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (FSSCP) methods. In SSCP, oligonucleotide primers derived from the polynucleotide sequences encoding TRFX are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (is SNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

Methods which may also be used to quantify the expression of TRFX include radiolabeling or biotinylating nucleotides, coamplification of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236.) The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotide sequences described herein may be used as elements on a microarray. The microarray can be used in transcript imaging techniques which monitor the relative expression levels of large numbers of genes simultaneously as described in Seilhamer, J. J. et al., “Comparative Gene Transcript Analysis,” U.S. Pat. No. 5,840,484, incorporated herein by reference. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, antibodies specific for TRFX, or TRFX or fragments thereof may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471, expressly incorporated by reference herein). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http://www.niehs.nih.gov/oc/news/toxchip.htm.) Therefore, it is important and desirable in toxicological screening using toxicant signatures to include all expressed gene sequences.

In one embodiment, the toxicity of a test compound is assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another particular embodiment relates to the use of the polypeptide sequences of the present invention to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of the present invention. In some cases, further sequence data may be obtained for definitive protein identification.

A proteomic profile may also be generated using antibodies specific for TRFX to quantify the levels of TRFX expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thio- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18-533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art. (See, e.g., Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Microarrays: A Practical Approach, M. Schena, ed. (1999) Oxford University Press, London, hereby expressly incorporated by reference.

In another embodiment of the invention, nucleic acid sequences encoding TRFX may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.) Once mapped, the nucleic acid sequences of the invention may be used to develop genetic linkage maps, for example, which correlate the inheritance of a disease state with the inheritance of a particular chromosome region or restriction fragment length polymorphism (RFLP). (See, e.g., Lander, E. S. and D. Botstein (1986) Proc. Natl. Acad. Sci. USA 83:7353-7357.)

Fluorescent in situ hybridization (FISH) may be correlated with other physical and genetic map data. (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding TRFX on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genornic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the instant invention may also be used to detect differences in the chromosomal location due to translocation, inversion, etc., among normal, carrier, or affected individuals.

In another embodiment of the invention, TRFX, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between TRFX and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with TRFX, or fragments thereof, and washed. Bound TRFX is then detected by methods well known in the art. Purified TRFX can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding TRFX specifically compete with a test compound for binding TRFX. In this manner, antibodies can be used to detect the presence of any peptide which shares one or more antigenic determinants with TRFX.

In additional embodiments, the nucleotide sequences which encode TRFX may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The disclosures of all patents, applications, and publications mentioned above and below, in particular U.S. Ser. No. 60/188,986, are hereby expressly incorporated by reference.

### EXAMPLES

I. Construction of cDNA Libraries

RNA was purchased from Clontech or isolated from tissues described in Table 4. Some tissues were homogenized and lysed in guanidinium isothiocyanate, while others were homogenized and lysed in phenol or in a suitable mixture of denaturants, such as TRIZOL (Life Technologies), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A+) RNA was isolated using oligo d(T)coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies), using the recommended procedures or similar methods known in the art. (See, e.g., Ausubel, 1997, supra, units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Pharmacia Biotech) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Life Technologies), pcDNA2.1 plasmid (Invitrogen, Carlsbad Calif.), or pINCY plasmid (Incyte Genomics, Palo Alto Calif.). Recombinant plasmids were transformed into competent E. coli cells including XL1-Blue, XL1-BlueMRF, or SOLR from Stratagene or DH5α, DH10B, or ElectroMAX DH10B from Life Technologies.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.AL. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384 well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Pharmacia Biotech or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Molecular Dynamics); the ABI PRISM 373 or 377 sequencing system (Applied Biosystems) in conjunction with standard ABI protocols and base calling software; or other sequence analysis systems known in the art. Reading frames within the cDNA sequences were identified using standard methods (reviewed in Ausubel, 1997, supra, unit 7.7). Some of the cDNA sequences were selected for extension using the techniques disclosed in Example VI.

The polynucleotide sequences derived from cDNA sequencing were assembled and analyzed using a combination of software programs which utilize algorithms well known to those skilled in the art. Table 5 summarizes the tools, programs, and algorithms used and provides applicable descriptions, references, and threshold parameters. The first column of Table 5 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score, the greater the homology between two sequences). Sequences were analyzed using MAcDNASIS PRO software (Hitachi Software Engineering, South San Francisco Calif.) and LASERGENE software (DNASTAR). Polynucleotide and polypeptide sequence alignments were generated using the default parameters specified by the clustal algorithm as incorporated into the MEGALIGN multisequence alignment program (DNASTAR), which also calculates the percent identity between aligned sequences.

The polynucleotide sequences were validated by removing vector, linker, and polyA sequences and by masking ambiguous bases, using algorithms and programs based on BLAST, dynamic programing, and dinucleotide nearest neighbor analysis. The sequences were then queried against a selection of public databases such as the GenBank primate, rodent, mammalian, vertebrate, and eukaryote databases, and BLOCKS, PRINTS, DOMO, PRODOM, and PFAM to acquire annotation using programs based on BLAST, FASTA, and BUMPS. The sequences were assembled into fill length polynucleotide sequences using programs based on Phred, Phrap, and Consed, and were screened for open reading frames using programs based on GeneMark, BLAST, and FASTA. The full length polynucleotide sequences were translated to derive the corresponding full length amino acid sequences, and these full length sequences were subsequently analyzed by querying against databases such as the GenBank databases (described above), SwissProt, BLOCKS, PRINTS, DOMO, PRODOM, Prosite, and Hidden Markov Model (HMM)-based protein family databases such as PFAM. HMM is a probabilistic approach which analyzes consensus primary structures of gene families. (See, e.g., Eddy, S. R. (1996) Curr. Opin. Struct. Biol. 6:361-365.)

The programs described above for the assembly and analysis of full length polynucleotide and amino acid sequences were also used to identify polynucleotide sequence fragments from SEQ ID NO:108-214. Fragments from about 20 to about 4000 nucleotides which are useful in hybridization and amplification technologies were described in The Invention section above.

IV. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel, 1995, supra, ch. 4 and 16.)

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq .1 ) , length ( Seq .2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and 4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

The results of northern analyses are reported as a percentage distribution of libraries in which the transcript encoding TRFX occurred. Analysis involved the categorization of cDNA libraries by organ/tissue and disease. The organ/tissue categories included cardiovascular, dermatologic, developmental, endocrine, gastrointestinal, hematopoietic/immune, musculoskeletal, nervous, reproductive, and urologic. The disease/condition categories included cancer, inflammation, trauma, cell proliferation, neurological, and pooled. For each category, the number of libraries expressing the sequence of interest was counted and divided by the total number of libraries across all categories. Percentage values of tissue-specific and disease- or condition-specific expression are reported in Table 3.

V. Chromosomal Mapping of TRFX Encoding Polynucleotides

The cDNA sequences which were used to assemble SEQ ID NO:108-214 were compared with sequences from the Incyte LIFESEQ database and public domain databases using BLAST and other implementations of the Smith-Waterman algorithm. Sequences from these databases that matched SEQ ID NO:108-214 were assembled into clusters of contiguous and overlapping sequences using assembly algorithms such as Phrap (Table 5). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

The genetic map locations of SEQ ID NO:111, 114, 117, 122, 123, 125, 130, 132-134, 136, 138, 145, 149, 152, 153, 156, 159, 168, 179, 180, 184, 185, 196, 197, 199, 201, 204, 208, 212, and 213, are described in The Invention as ranges, or intervals, of human chromosomes. More than one map location is reported for SEQ ID NO:145, 152, 184, 185, 199, 208, and 212, indicating that previously mapped sequences having similarity, but not complete identity, to SEQ ID NO:145, 152, 184, 185, 199, 208, and 212 were assembled into their respective clusters. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VI. Extension of TRFX Encoding Polynucleotides

The full length nucleic acid sequences of SEQ ID NO:108-214 were produced by extension of an appropriate fragment of the full length molecule using oligonucleotide primers designed from this fragment. One primer was synthesized to initiate 5′ extension of the known fragment, and the other primer, to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-priner dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and β-mercaptoethanol, Taq DNA polymerase (Amersham Pharmacia Biotech), ELONGASE enzyme (Life Technologies), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Pharmacia Biotech). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Phanracia Biotech), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Pharmacia Biotech) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Pharmacia Biotech) or the ABI PRISM BIGDYE Terminator cycle sequencing ready reaction kit (Applied Biosystems).

In like manner, the polynucleotide sequences of SEQ ID NO:108-214 are used to obtain 5′ regulatory sequences using the procedure above, along with oligonucleotides designed for such extension, and an appropriate genomic library.

VII. Labeling and Use of Individual Hybridization Probes

Hybridization probes derived from SEQ ID NO:108-214 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Pharmacia Biotech), and T4 polynucleotide kinase (DuPont NEN, Boston Mass.). The labeled oligonucleotides are substantially purified using a SEPHADEX G-25 superfime size exclusion dextran bead column (Amersham Pharmacia Biotech). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

VIII. Microarrays

The linkage or synthesis of array elements upon a microarray can be achieved utilizing photolithography, piezoelectric printing (ink-jet printing, See, e.g., Baldeschweiler, supra), mechanical microspotting technologies, and derivatives thereof. The substrate in each of the aforementioned technologies should be uniform and solid with a non-porous surface (Schena (1999), supra). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements. (See, e.g., Schena, M. et al. (1995) Science 270:467-470; Shalon, D. et al. (1996) Genome Res. 6:639-645; Marshall, A. and J. Hodgson (1998) Nat. Biotechnol. 16:27-31.)

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21mer), 1× first strand buffer, 0.03 units/μl RNase inhibitor, 500 μM DATP, 500 μM dGTP, 500 μM dTTP, 40 μM dCTP, 40 μM dCTP-Cy3 (BDS) or dCTP-Cy5 (Amersham Pharmacia Biotech). The reverse transcription reaction is performed in a 25 ml volume containing 200 ng poly(A)⁺ RNA with GEMBRIGHT kits (Incyte). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTICH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Pharmacia Biotech).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crossllnked using a STRATALINER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of CyS. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit Rn-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte).

IX. Complementary Polynucleotides

Sequences complementary to the TRFX-encoding sequences, or any parts thereof, are used to detect, decrease, or inhibit expression of naturally occurring TRFX. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of TRFX. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the TRFX-encoding transcript.

X. Expression of TRFX

Expression and purification of TRFX is achieved using bacterial or virus-based expression systems. For expression of TRFX in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express TRFX upon induction with isopropyl beta-D-thiogalactopyranoside (IPTG). Expression of TRFX in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding TRFX by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugirerda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, TRFX is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma iaponicum, enables the purification of fusion proteins on immobilized glutathione under conditions that maintain protein activity and antigenicity (Amersham Pharmacia Biotech). Following purification, the GST moiety can be proteolytically cleaved from TRFX at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Purified TRFX obtained by these methods can be used directly in the assays shown in Examples XI and XV.

XI. Demonstration of TRFX Activity

TRFX activity is measured by its ability to stimulate transcription of a reporter gene (Liu, H. Y. et al. (1997) EMBO J. 16(17):5289-5298). The assay entails the use of a well characterized reporter gene construct, LexAₒₚ-LacZ, that consists of LexA DNA transcriptional control elements (Lex) fused to sequences encoding the E. coli LacZ enzyme. The methods for constructing and expressing fusion genes, introducing them into cells, and measuring LacZ enzyme activity, are well known to those skilled in the art. Sequences encoding TRFX are cloned into a plasmid that directs the synthesis of a fusion protein, LexA-TRFX, consisting of TRFX and a DNA binding domain derived from the LexA transcription factor. The resulting plasmid, encoding a LexA-TRFX fusion protein, is introduced into yeast cells along with a plasmid containing the LexAₒₚ-LacZ reporter gene. The amount of LacZ enzyme activity associated with LexA-TRFX transfected cells, relative to control cells, is proportional to the amount of transcription stimulated by the TRFX.

XII. Functional Assays

TRFX function is assessed by expressing the sequences encoding TRFX at physiologically elevated levels in mammalian cell culture systems. cDNA is subcloned into a mammalian expression vector containing a strong promoter that drives high levels of cDNA expression. Vectors of choice include pCMV SPORT plasmid (Life Technologies) and pCR3.1 plasmid (Invitrogen), both of which contain the cytomegalovirus promoter. 5-10 μg of recombinant vector are transiently transfected into a human cell line, for example, an endothelial or hematopoietic cell line, using either liposome, formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64GFP fusion protein. Flow cytometry (FCM), an automated, laser optics-based technique, is used to identify transfected cells expressing GFP or CD64GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulaiion of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of TRFX on gene expression can be assessed using highly purified populations of cells transfected with sequences encoding TRFX and either CD64 or CD64GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding TRFX and other genes of interest can be analyzed by northern analysis or microarray techniques.

XIII. Production of TRFX Specific Antibodies

TRFX substantially purified using polyacrylamide gel electrophoresis (PAGE; see, e.g., Harrington, M. G. (1990) Methods Enzymol. 182:488495), or other purification techniques, is used to immunize rabbits and to produce antibodies using standard protocols.

Alternatively, the TRFX amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art. (See, e.g., Ausubel, 1995, supra, ch. 11.)

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenwyl-N-hydroxysuccinimide ester (MBS) to increase immunogenicity. (See, e.g., Ausubel, 1995, surra.) Rabbits are immunized with the oligopeptide-KLH complex in complete Freund's adjuvant. Resulting antisera are tested for antipeptide and anti-TRFX activity by, for example, binding the peptide or TRFX to a substrate, blocking with 1% BSA, reacting with rabbit antisera, washing, and reacting with radio-iodinated goat anti-rabbit IgG.

XIV. Purification of Naturally Occurring TREX Using Specific Antibodies

Naturally occurring or recombinant TRFX is substantially purified by immunoaffinity chromatography using antibodies specific for TRFX. An immunoaffinity column is constructed by covalently coupling anti-TRFX antibody to an activated chromatographic resin, such as CNBr-activated SEPHAROSE (Amersham Pharmacia Biotech). After the coupling, the resin is blocked and washed according to the manufacturer's instructions.

Media containing TRFX are passed over the immunoaffinity column, and the column is washed under conditions that allow the preferential absorbance of TRFX (e.g., high ionic strength buffers in the presence of detergent). The column is eluted under conditions that disrupt antibody/TRFX binding (e.g., a buffer of pH 2 to pH 3, or a high concentration of a chaotrope, such as urea or thiocyanate ion), and TRFX is collected.

XV. Identification of Molecules which Interact with TRFX

TRFX, or biologically active fragments thereof, are labeled with ¹²⁵I Bolton-Hunter reagent. (See, e.g., Bolton A. E. and W. M. Hunter (1973) Biochem. J. 133:529-539.) Candidate molecules previously arrayed in the wells of a multi-well plate are incubated with the labeled TRFX, washed, and any wells with labeled TRFX complex are assayed. Data obtained using different concentrations of TRFX are used to calculate values for the number, affinity, and association of TREX with the candidate molecules.

Alternatively, molecules interacting with TRFX are analyzed using the yeast two-hybrid system as described in Fields, S. and 0. Song (1989, Nature 340:245-246), or using commercially available kits based on the two-hybrid system, such as the MATCH ER system (Clontech).

TRFX may also be used in the PATHCAILING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

Various modifications and variations of the described methods and systems of the invention will be apparent to those skilled in the art without departing from the scope and spirit of the invention. Although the invention has been described in connection with certain embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1

| Polypeptide | Nucleotide | Clone | | |
|---|---|---|---|---|
| SEQ ID NO: | SEQ ID NO: | ID | Library | Fragments |
| 1 | 108 | 095210 | PITUNOT01 | 095210H1 (PITUNOT01), 095210R1 (PITUNOT01), 450088R1 (TLYMNOT02), |
| | | | | 1405954F6 (LATRTUT02), 1676067F6 (BLADNOT05), 3421076H1 (UCMCNOT04), |
| | | | | 3519949H1 (LUNGNON03), 3535670H1 (KIDNNOT25) |
| 2 | 109 | 157953 | THP1PLB02 | 157953F1 (THP1PLB02), 157953H1 (THP1PLB02), 279935H1 (LIVRNOT02), |
| | | | | 293820X24 (LIVRNOT04), 1210577R7 (BRSTNOT02), 2563416H1 (ADRETUT01), |
| | | | | 5049562H1 (BRSTNOT33), g678942 |
| 3 | 110 | 159196 | ADENINB01 | 159196H1 (ADENINB01), 873479R1 (LUNGAST01), 1695224F6 (COLNNOT23), |
| | | | | 4408025F6 (PROSDIT01), 4663865T6 (MEGBUNT01) |
| 4 | 111 | 343338 | THYMNOT02 | 343338H1 (THYMNOT02), 343338R6 (THYMNOT02), 343338T6 (THYMNOT02), |
| | | | | 1448112F6 (PLACNOT02), 1448112R1 (PLACNOT02), 2235177X14F1 (PANCTUT02), |
| | | | | 2235177X16F1 (PANCTUT02), 2235177X17F1 (PANCTUT02), 2241778F6 |
| | | | | (PANCTUT02), 2241778T6 (PANCTUT02), 2729457F6 (OVARTUT05), 4053846F6 |
| | | | | (SPLNNOT13), SBCA04252F1 |
| 5 | 112 | 402386 | TMLR3DT01 | 402386H1 (TMLR3DT01), 402386X11 (TMLR3DT01), 568243R1 (MMLR3DT01), |
| | | | | 568243T6 (MMLR3DT01), 731436H1 (LUNGNOT03), SAGA00508R1, SAGA00557R1 |
| 6 | 113 | 456487 | KERANOT01 | 168091H1 (LIVRNOT01), 456487H1 (KERANOT01), 532096R1 (BRAINOT03), |
| | | | | 619791H1 (PGANNOT01), 825933R1 (PROSNOT06), 1436382F1 (PANCNOT08), |
| | | | | 1439054F6 (PANCNOT08), 1700156F6 (BLADTUT05), 2274307R6 (PROSNON01), |
| | | | | 2515549H1 (LIVRTUT04), 5158675H1 (BRSTTMT02) |
| 7 | 114 | 490256 | HNT2AGT01 | 490256H1 (HNT2AGT01), 507309F1 (TMLR3DT02), 507309X15 (TMLR3DT02); |
| | | | | 2724951H1 (OVARTUT05), SZZZ00188R1, SZZZ02099R1, g825070, g1242173 |
| 8 | 115 | 494740 | HNT2NOT01 | 494740H1 (HNT2NOT01), 770196R1 (COLNCRT01), 1235126F1 (LUNGFET03), |
| | | | | 1235126T1 (LUNGFET03), 1326711F1 (LPARNOT02), 1816820F6 (PROSNOT20), |
| | | | | 1853059H1 (LUNGFET03) |
| 9 | 116 | 507475 | TMLR3DT02 | 507475H1 (TMLR3DT02), 535932R6 (ADRENOT03), 779955H1 (MYOMNOT01), |
| | | | | 1928396T6 (BRSTNOT02), 2078558H1 (ISLTNOT01) |
| 10 | 117 | 531581 | BRAINOT03 | 084009X13 (HYPONOB01), 413718R1 (BRSTNOT01), 413718X22F1 (BRSTNOT01), |
| | | | | 531581H1 (BRAINOT03), 531581T6 (BRAINOT03), 2171348H1 (ENDCNOT03), |
| | | | | 2795710T6 (NPOLNOT01), 2926562F7 (TLYMNOT04), 2926562T7 (TLYMNOT04), |
| | | | | 4341890H1 (BRAUNOT02), 4405904H1 (PROSDIT01) |
| 11 | 118 | 675190 | CRBLNOT01 | 675190H1 (CRBLNOT01), 675190X13 (CRBLNOT01), 1812672F6 (PROSTUT12), |
| | | | | 2573205R6 (HIPOAZT01) |
| 12 | 119 | 685434 | UTRSNOT02 | 685434CT1 (UTRSNOT02), 685434H1 (UTRSNOT02), 1904155F6 (OVARNOT07), |
| | | | | 2784031F6 (BRSTNOT13), 3129338F6 (LUNGTUT12) |
| 13 | 120 | 788663 | PROSNOT05 | 788663H1 (PROSNOT05), 1451931F1 (PENITUT01), 1960289H1 (BRSTNOT04), |
| | | | | 2083142F6 (UTRSNOT08), 2542122H1 (BONRTUT01), 2581153F6 (KIDNTUT13), |
| | | | | 3577780H1 (BRONNOT01) |
| 14 | 121 | 870100 | LUNGAST01 | 625091R6 (PGANNOT01), 870100H1 (LUNGAST01), 870100X12 (LUNGAST01), |
| | | | | 1231358H1 (BRAITUT01), STEQ00206R1, SZZZ00601R1, SZZZ02045R1 |
| 15 | 122 | 879500 | THYRNOT02 | 281880H1 (CARDNOT01), 859232R1 (BRAITUT03), 879500H1 (THYRNOT02), |
| | | | | 1250675F6 (LUNGFET03), 1601165F6 (BLADNOT03), 1823981F6 (GBLATUT01), |
| | | | | 2187360F6 (PROSNOT26), 2262956H1 (UTRSNOT02), 2433567H1 (BRAVUNT02), |
| | | | | 2656846F6 (LUNGTUT09), 2993077F6 (KIDNFET02), 3085846H1 (HEAONOT03), |
| | | | | 3181794T6 (TLYJNOT01), 4285141F6 (LIVRDIR01), 4774779H1 (BRAQNOT01), |
| | | | | 5507484H1 (BRADDIR01), 5512965H1 (BRADDIR01), SCMA05658V1, SCMA03540V1, |
| | | | | SCMA00007V1, g2224558 |
| 16 | 123 | 975377 | MUSCNOT02 | 026851R1 (SPLNFET01), 786313R1 (PROSNOT05), 975377H1 (MUSCNOT02), |
| | | | | 975377X19 (MUSCNOT02), 975377X21 (MUSCNOT02), 1354139X14 (LUNGNOT09), |
| | | | | 2546208H1 (UTRSNOT11) |
| 17 | 124 | 1208721 | BRSTNOT02 | 1208721H1 (BRSTNOT02), 1286769F1 (BRAINOT11), 1456447F6 (COLNFET02), |
| | | | | 1722840T6 (BLADNOT06), 1998475R6 (BRSTTUT03), 2740916F6 (BRSTTUT14), |
| | | | | 3234886H1 (COLNUCT03), 4588959H1 (MASTTXT01), 4710080H1 (BRAIFET02), |
| | | | | g1425135 |
| 18 | 125 | 1234329 | LUNGFET03 | 259818T6 (HNT2RAT01), 264365H1 (HNT2AGT01), 349606H1 (LVENNOT01), |
| | | | | 399059H1 (PITUNOT02), 1234329H1 (LUNGFET03), 1257012F1 (MENITUT03), |
| | | | | 1442838F1 (THYRNOT03), 1443014R1 (THYRNOT03), 1515850F1 (PANCTUT01), |
| | | | | 2186886F6 (PROSNOT26), 2655641F6 (THYMNOT04), 2703809F6 (OVARTUT10), |
| | | | | g1688736, g1985577 |
| 19 | 126 | 1238747 | LUNGTUT02 | 501158R6 (NEUTLPT01), 565047H1 (NEUTLPT01), 769541R6 (COLNCRT01), |
| | | | | 890561T6 (STOMTUT01), 1238747H1 (LUNGTUT02), 1510233F6 (LUNGNOT14), |
| | | | | 1510233T6 (LUNGNOT14) |
| 20 | 127 | 1265980 | BRAINOT09 | 1265980H1 (BRAINOT09), 2155287X13F1 (BRAINOT09), 2155287X23F1 |
| | | | | (BRAINOT09), 2158376T6 (BRAINOT09), SAGA02430F1 |
| 21 | 128 | 1297333 | BRSTNOT07 | 1297333H1 (BRSTNOT07), 1297333X12 (BRSTNOT07), 1297333X14 (BRSTNOT07), |
| | | | | SAGA00259F1, SAGA00400R1 |
| 22 | 129 | 1312824 | BLADTUT02 | 306400R6 (HEARNOT01), 1312824H1 (BLADTUT02), 1312824T6 (BLADTUT02), |
| | | | | 1840110H1 (EOSITXT01), 1846489R6 (COLNNOT09), 1985201R6 (LUNGAST01), |
| | | | | 2199162H1 (SPLNFET02), 2779784H1 (OVARTUT03), 3528903H1 (BLADNOT09), |
| | | | | 3767951H1 (BRSTNOT24), 4251647H1 (BRADDIR01), 5205078H2 (BRAFNOT02), |
| | | | | 5423679H1 (PROSTMT07), SANA02095F1, g1941058 |
| 23 | 130 | 1359294 | LUNGNOT12 | 139446H1 (LIVRNOT01), 258759H1 (HNT2RAT01), 268845H1 (HNT2NOT01), |
| | | | | 492813R1 (HNT2NOT01), 1213691H1 (BRSTTUT01), 1222480H1 (COLNTUT02), |
| | | | | 1243093H1 (LUNGNOT03), 1319296H1 (BLADNOT04), 1359294H1 (LUNGNOT12), |
| | | | | 1404752F6 (LATRTUT02), 1404752T6 (LATRTUT02), 1479678H1 (CORPNOT02), |
| | | | | 1558471H1 (SPLNNOT04), 1857126H1 (PROSNOT18), 1870761H1 (SKINBIT01) |
| 24 | 131 | 1377380 | LUNGNOT10 | 962085R1 (BRSTTUT03), 1377380H1 (LUNGNOT10), 1670530F6 (BMARNOT03), |
| | | | | 1853551T6 (LUNGFET03), 2119555R6 (BRSTTUT02), SCIA03178V1 |
| 25 | 132 | 1383473 | BRAITUT08 | 780421H1 (MYOMNOT01), 1344946F6 (PROSNOT11), 1383473F6 (BRAITUT08), |
| | | | | 1383473H1 (BRAITUT08), 1906164T6 (OVARNOT07), 2302122R6 (BRSTNOT05), |
| | | | | 2328233R6 (COLNNOT11), 2615335F6 (GBLANOT01), 5836742H1 (BRAIDIT05) |
| 26 | 133 | 1388860 | EOSINOT01 | 415763R1 (BRSTNOT01), 1388860H1 (EOSINOT01), SAFC02379F1, SAFC01030F1, |
| | | | | SAFC00771F1, SAFC02719F1 |
| 27 | 134 | 1395322 | THYRNOT03 | 1332909F6 (PANCNOT07), 1332909X16 (PANCNOT07), 1332909X23R1 (PANCNOT07), |
| | | | | 1332909X24R1 (PANCNOT07), 1395322H1 (THYRNOT03), 1477406F1 (CORPNOT02), |
| | | | | 3422017H1 (UCMCNOT04) |
| 28 | 135 | 1419370 | KIDNNOT09 | 243596H1 (HIPONOT01), 929439R1 (CERVNOT01), 1310519F1 (COLNFET02), |
| | | | | 1395856T1 (THYRNOT03), 1419370F1 (KIDNNOT09), 1419370H1 (KIDNNOT09), |
| | | | | 1666159F6 (BRSTNOT09), 3461531H1 (293TF2T01), 4710948H1 (BRAIFET02), |
| | | | | SBGA01870F1, g947108, g1991693 |
| 29 | 136 | 1429773 | SINTBST01 | 1306171T6 (PLACNOT02), 1313558F1 (BLADTUT02), 1429773H1 (SINTBST01), |
| | | | | 1469411F1 (PANCTUT02), 1626615F6 (COLNPOT01), 1807088F6 (SINTNOT13), |
| | | | | 2641613F6 (LUNGTUT08), 2692245F6 (LUNGNOT23), 2695323H1 (UTRSNOT12), |
| | | | | 2851378H1 (BRSTTUT13), 3387328F6 (LUNGTUT17) |
| 30 | 137 | 1470820 | PANCTUT02 | 1232690F6 (LUNGFET03), 1470820H1 (PANCTUT02), 1484705F1 (CORPNOT02), |
| | | | | 2831707F6 (TLYMNOT03), 3073715H1 (BONEUNT01) |
| 31 | 138 | 1483455 | CORPNOT02 | 487811X26 (HNT2AGT01), 1483455H1 (CORPNOT02), 1849167F6 (LUNGFET03), |
| | | | | 1856220F6 (PROSNOT18), 2822949F6 (ADRETUT06), 2822949T6 (ADRETUT06), |
| | | | | 2851743F6 (BRSTTUT13), g2159610 |
| 32 | 139 | 1527064 | UCMCL5T01 | 001612H1 (U937NOT01), 001923H1 (U937NOT01), 1235664H1 (LUNGFET03), |
| | | | | 1412779H1 (BRAINOT12), 1527064H1 (UCMCL5T01), 1598233T6 (BLADNOT03), |
| | | | | 1702565H1 (BLADTUT05), 1973691H1 (UCMCL5T01), 2227436H1 (SEMVNOT01), |
| | | | | 2472092F6 (THP1NOT03), 2634126H1 (COLNTUT15) |
| 33 | 140 | 1557491 | BLADTUT04 | 046771H1 (CORNNOT01), 1456684F6 (COLNFET02), 1456684T6 (COLNFET02), |
| | | | | 1554967F1 (BLADTUT04), 1557491H1 (BLADTUT04), 1992143H1 (CORPNOT02), |
| | | | | 2687476F6 (LUNGNOT23), 3139175F6 (SMCCNOT02), 4746319H1 (SMCRUNT01) |
| 34 | 141 | 1576862 | LNODNOT03 | 496787F1 (HNT2NOT01), 496787R1 (HNT2NOT01), 1572855F6 (LNODNOT03), |
| | | | | 1576862H1 (LNODNOT03), 1576862X11 (LNODNOT03), 1576862X19 (LNODNOT03), |
| | | | | 1576862X21 (LNODNOT03), 3284579T6 (HEAONOT05), SBIA03851D1, SBIA04892D1, |
| | | | | SBIA07089D1 |
| 35 | 142 | 1609731 | COLNTUT06 | 112132F1 (PITUNOT01), 112132R1 (PITUNOT01), 159643X1 (ADENINB01), |
| | | | | 1609731H1 (COLNTUT06), 1609731T6 (COLNTUT06), 5445363H1 (LNODNOT12), |
| | | | | g2204797 |
| 36 | 143 | 1674538 | BLADNOT05 | 1432420H1 (BEPINON01), 1579336F6 (DUODNOT01), 1674538F6 (BLADNOT05), |
| | | | | 1674538H1 (BLADNOT05), 2656555H1 (LUNGTUT09), 4249348H1 (BRADDIR01), |
| | | | | 4618275H1 (BRAYDIT01), 4760417H1 (BRAMNOT01), g3785154, g1623216, |
| | | | | g899854, g1717534 |
| 37 | 144 | 1675287 | BLADNOT05 | 868686T1 (LUNGAST01), 984876R1 (LVENNOT03), 1456253F1 (COLNFET02), |
| | | | | 1675287H1 (BLADNOT05), 1675845H1 (BLADNOT05), 2047281F6 (THP1T7T01), |
| | | | | 2808537H1 (BLADTUT08), 4883514F6 (LUNLTMT01) |
| 38 | 145 | 1693903 | COLNNOT23 | 1358877F1 (LUNGNOT09), 1573956F1 (LNODNOT03), 1693903F6 (COLNNOT23), |
| | | | | 1693903H1 (COLNNOT23), 2184065F6 (SININOT01), 3316112F6 (PROSBPT03), |
| | | | | SXAF02294V1 |
| 39 | 146 | 1702962 | DUODNOT02 | 794279R6 (OVARNOT03), 814285R6 (OVARTUT01), 1702962H1 (DUODNOT02), |
| | | | | 2186132H1 (PROSNOT26), 2880019F6 (UTRSTUT05), 5196364H1 (LUNLTUT04) |
| 40 | 147 | 1712916 | PROSNOT16 | 1712916F6 (PROSNOT16), 1712916H1 (PROSNOT16), 2186575F6 (PROSNOT26), |
| | | | | g3399946 |
| 41 | 148 | 1748313 | STOMTUT02 | 940469R6 (ADRENOT03), 1317481F6 (BLADTUT02), 1748313H1 (STOMTUT02), |
| | | | | 1870549F6 (SKINBIT01), 2169544F6 (ENDCNOT03), 2285816H1 (BRAINON01), |
| | | | | 2383066F6 (ISLTNOT01), 2613757F6 (ESOGTUT02), SZAS01459V1, SZAS00220V1 |
| 42 | 149 | 1754833 | LIVRTUT01 | 710767H1 (SYNORAT04), 1396892F6 (BRAITUT08), 1754833H1 (LIVRTUT01), |
| | | | | 1754833T6 (LIVRTUT01), 1879592F6 (LEUKNOT03), 2331424R6 (COLNNOT11), |
| | | | | 3125146H1 (LNODNOT05), 3212201H1 (BLADNOT08), 3585117H1 (293TF4T01) |
| 43 | 150 | 1798701 | COLNNOT27 | 122777F1 (LUNGNOT01), 122777R1 (LUNGNOT01), 1215026R6 (BRSTTUT01), |
| | | | | 1753224H1 (LIVRTUT01), 1798701H1 (COLNNOT27), 2041087H1 (HIPONON02), |
| | | | | SAEA00596F1, SAEA00135F1 |
| 44 | 151 | 1842496 | COLNNOT07 | 027249F1 (SPLNFET01), 1330406H1 (PANCNOT07), 1842496H1 (COLNNOT07), |
| | | | | 1981256R6 (LUNGTUT03), 3215321F7 (TESTNOT07) |
| 45 | 152 | 1868613 | SKINBIT01 | 1868613H1 (SKINBIT01), 1999115R6 (BRSTTUT03), 2159835F7 (ENDCNOT02), |
| | | | | 2453392H1 (ENDANOT01), 2753832H1 (THP1AZS08), 2781021T6 (OVARTUT03), |
| | | | | 3597161F6 (FIBPNOT01), 4567678H1 (HELATXT01), 4998328H1 (MYEPTXT02) |
| 46 | 153 | 1870609 | SKINBIT01 | 474617H1 (MMLR1DT01), 1391829F6 (THYRNOT03), 1722968F6 (BLADNOT06), |
| | | | | 1722968T6 (BLADNOT06), 1833131H1 (BRAINON01), 1870609F6 (SKINBIT01), |
| | | | | 1870609H1 (SKINBIT01), 1870609T6 (SKINBIT01), 2542675H2 (UTRSNOT11), |
| | | | | 2580351F6 (KIDNTUT13), 2653740H1 (THYMNOT04), 3228774H1 (COTRNOT01) |
| 47 | 154 | 1871961 | LEUKNOT02 | 743684F1 (BRAITUT01), 835705R1 (PROSNOT07), 1624519F6 (BRAITUT13), |
| | | | | 1688618F6 (PROSTUT10), 1871961F6 (LEUKNOT02), 1871961H1 (LEUKNOT02), |
| | | | | 1965802R6 (BRSTNOT04), 2453823F6 (ENDANOT01), 4689940H1 (PROSTMT05) |
| 48 | 155 | 1876258 | LEUKNOT02 | 808836R1 (LUNGNOT04), 1390870H1 (EOSINOT01), 1876258H1 (LEUKNOT02), |
| | | | | SZAH00430F1, SZAH03995F1, SZAH00534F1, SZAH01526F1 |
| 49 | 156 | 1929822 | COLNTUT03 | 040201F1 (TBLYNOT01), 424589R6 (BLADNOT01), 638245H1 (BRSTNOT03), |
| | | | | 1251025F1 (LUNGFET03), 1391470H1 (EOSINOT01), 1699535F6 (BLADTUT05), |
| | | | | 1929822H1 (COLNTUT03), 2218644H1 (LUNGNOT18), 2291751R6 (BRAINON01), |
| | | | | 3242060H1 (COLAUCT01), 3317796F6 (PROSBPT03), 3401711H1 (ESOGNOT03), |
| | | | | 3488355H1 (EPIGNOT01), 4030773H1 (BRAINOT23), 4180362H1 (SINITUT03), |
| | | | | 4891448H1 (PROSTMT05), 5539034H1 (KIDNFEC01), g3882288 |
| 50 | 157 | 1970095 | UCMCL5T01 | 114097F1 (TESTNOT01), 168757H1 (LIVRNOT01), 754038R1 (BRAITUT02), |
| | | | | 772953R1 (COLNCRT01), 880261R1 (THYRNOT02), 1970095F6 (UCMCL5T01), |
| | | | | 1970095H1 (UCMCL5T01), 2235148F6 (PANCTUT02), SAEA02374R1 |
| 51 | 158 | 1975473 | PANCTUT02 | 1340447F1 (COLNTUT03), 1500133F6 (SINTBST01), 1663908F6 (BRSTNOT09), |
| | | | | 1975473H1 (PANCTUT02), 3726008H1 (BRSTNOT23) |
| 52 | 159 | 1976527 | PANCTUT02 | 160328R6 (ADENINB01), 982222T2 (TONGTUT01), 993118R6 (COLNNOT11), |
| | | | | 1709642T6 (PROSNOT16), 1976527F6 (PANCTUT02), 1976527H1 (PANCTUT02), |
| | | | | 3586151F6 (293TF4T01), SXAE03918V1, SXAE05371V1 |
| 53 | 160 | 2108023 | BRAITUT03 | 1493429H1 (PROSNON01), 2012466H1 (TESTNOT03), 2108023H1 (BRAITUT03), |
| | | | | 2108023T6 (BRAITUT03) |
| 54 | 161 | 2135746 | ENDCNOT01 | 998857R6 (KIDNTUT01), 1384325F1 (BRAITUT08), 1727569F6 (PROSNOT14), |
| | | | | 2135746F6 (ENDCNOT01), 2135746H1 (ENDCNOT01), 2255539R6 (OVARTUT01), |
| | | | | 2999008H1 (OVARTUT07), 3623266H1 (ENDANOT03), 5412531H1 (BRATNOT03) |
| 55 | 162 | 2154810 | BRAINOT09 | 035033X11 (HUVENOB01), 035033X14 (HUVENOB01), 1857664F6 (PROSNOT18), |
| | | | | 1857664T6 (PROSNOT18), 2154810H1 (BRAINOT09), 2847166T6 (DRGLNOT01), |
| | | | | 5094009F6 (EPIMNON05) |
| 56 | 163 | 2228991 | PROSNOT16 | 2228991F6 (PROSNOT16), 2228991H1 (PROSNOT16), 3970066F6 (PROSTUT10) |
| 57 | 164 | 2241206 | PANCTUT02 | 1235632F6 (LUNGFET03), 1514392F1 (PANCTUT01), 1533915F1 (SPLNNOT04), |
| | | | | 2241206H1 (PANCTUT02), 2724267X303D1 (LUNGTUT10), 5218584T6 (BRSTNOT35), |
| | | | | 5567773H1 (TLYMNOT08) |
| 58 | 165 | 2259590 | OVARTUT01 | 935085T1 (CERVNOT01), 1915979H1 (PROSTUT04), 2259590H1 (OVARTUT01), |
| | | | | 2259590R6 (OVARTUT01), 2259590T6 (OVARTUT01) |
| 59 | 166 | 2307537 | NGANNOT01 | 628086T6 (KIDNNOT05), 931221T6 (CERVNOT01), 2307537H1 (NGANNOT01), |
| | | | | 2307537R6 (NGANNOT01), 2799812H1 (PENCNOT01), 3318983H1 (PROSBPT03), |
| | | | | 4158531F6 (ADRENOT14), SBZA00461V1, SBZA04079V1 |
| 60 | 167 | 2440675 | EOSITXT01 | 806660R6 (BSTMNOT01), 1390870H1 (EOSINOT01), 2440675H1 (EOSITXT01), |
| | | | | SZAH00430F1, SZAH03995F1, SZAH00534F1, SZAH01526F1 |
| 61 | 168 | 2463542 | THYRNOT08 | 2463542F6 (THYRNOT08), 2463542H1 (THYRNOT08), 2552885F6 (THYMNOT03), |
| | | | | 2655535F7 (THYMNOT04), 2869957T6 (THYRNOT10), 3042074F7 (BRSTNOT16), |
| | | | | 3769037H1 (BRSTNOT24), 3801333H1 (SPLNNOT12), 3927329H1 (KIDNNOT19) |
| 62 | 169 | 2486031 | CONUTUT01 | 1417222F6 (BRAINOT12), 2486031F6 (CONUTUT01), 2486031H1 (CONUTUT01), |
| | | | | 2634120X315D2 (COLNTUT15), 2951631T6 (KIDNFET01), g3806506 |
| 63 | 170 | 2493052 | ADRETUT05 | 1376888T6 (LUNGNOT10), 1488851F6 (UCMCL5T01), 2108437R6 (BRAITUT03), |
| | | | | 2493052F7 (ADRETUT05), 2493052H1 (ADRETUT05), 2493052T6 (ADRETUT05), |
| | | | | 2840241F6 (DRGLNOT01), 4364312H1 (SKIRNOT01) |
| 64 | 171 | 2512074 | CONUTUT01 | 008250X12 (HMC1NOT01), 030534X12 (THP1NOB01), 1686214F6 (PROSNOT15), |
| | | | | 2395458F6 (THP1AZT01), 2512074H1 (CONUTUT01), 2963912F6 (SCORNOT04), |
| | | | | 5326933F6 (DRGTNON04) |
| 65 | 172 | 2646274 | LUNGTUT11 | 724811R6 (SYNOOAT01), 2646274H1 (LUNGTUT11), 3436027F6 (PENCNOT05) |
| 66 | 173 | 2672566 | KIDNNOT19 | 1381053F1 (BRAITUT08), 2108293R6 (BRAITUT03), 2672566H1 (KIDNNOT19), |
| | | | | 2908546F6 (THYMNOT05), 3730092H1 (SMCCNON03) |
| 67 | 174 | 2689674 | LUNGNOT23 | 2256960T6 (OVARTUT01), 2507571F6 (CONUTUT01), 2689674F6 (LUNGNOT23), |
| | | | | 2689674H1 (LUNGNOT23), 2755742H1 (THP1AZS08), 5096438H1 (EPIMNON05) |
| 68 | 175 | 2703282 | OVARTUT10 | 056400H1 (FIBRNOT01), 1484887F6 (CORPNOT02), 1484887T6 (CORPNOT02), |
| | | | | 1641813F6 (HEARFET01), 1810188H1 (PROSTUT12), 2351291F6 (COLSUCT01), |
| | | | | 2703282H1 (OVARTUT10), 3790456H1 (BRSTNOT28), 4084543T6 (CONFNOT02), |
| | | | | 4994160H1 (LIVRTUT11), 5393763H1 (KIDNNOT32) |
| 69 | 176 | 2738293 | OVARNOT09 | 412176R1 (BRSTNOT01), 418633T6 (BRSTNOT01), 1232594F1 (LUNGFET03), |
| | | | | 1301651T6 (BRSTNOT07), 2738293F6 (OVARNOT09), 2738293H1 (OVARNOT09), |
| | | | | 5290883H1 (LIVRTUS02) |
| 70 | 177 | 2772776 | PANCNOT15 | 784334R1 (PROSNOT05), 2772776H1 (PANCNOT15), 3750404H1 (UTRSNOT18) |
| 71 | 178 | 2774476 | PANCNOT15 | 2774476H1 (PANCNOT15), 3664676T6 (PANCNOT16), 3835889F6 (PANCNOT17), |
| | | | | 4167883X305V1 (PANCNOT21), SCCA02152V1 |
| 72 | 179 | 2804624 | BLADTUT08 | 162435R1 (ADENINB01), 1304830T1 (PLACNOT02), 2080378X19F1 (UTRSNOT08), |
| | | | | 2660596H1 (LUNGTUT09), 2804624H1 (BLADTUT08) |
| 73 | 180 | 2848225 | BRSTTUT13 | 346073X101 (THYMNOT02), 346073X26C1 (THYMNOT02), 391609T6 (TMLR2DT01), |
| | | | | 2848225H1 (BRSTTUT13), 4624612T6 (ENDVNOT01) |
| 74 | 181 | 2882241 | UTRSTUT05 | 1637060F6 (UTRSNOT06), 1711682F6 (PROSNOT16), 1902475H1 (OVARNOT07), |
| | | | | 2017387F6 (THP1NOT01), 2882241F6 (UTRSTUT05), 2882241H1 (UTRSTUT05), |
| | | | | 3532864H1 (KIDNNOT25) |
| 75 | 182 | 2939011 | THYMFET02 | 897237R1 (BRSTNOT05), 897237T1 (BRSTNOT05), 1618381F6 (BRAITUT12), |
| | | | | 2679105F6 (SINIUCT01), 2939011F6 (THYMFET02), 2939011H1 (THYMFET02), |
| | | | | 2939011T6 (THYMFET02) |
| 76 | 183 | 2947188 | BRAITUT23 | 377292X1 (NEUTFMT01), 425953R6 (BLADNOT01), 425953T6 (BLADNOT01), |
| | | | | 425953X28 (BLADNOT01), 429350T6 (BLADNOT01), 451192F1 (TLYMNOT02), |
| | | | | 451192R1 (TLYMNOT02), 1786579H1 (BRAINOT10), 2947188H1 (BRAITUT23) |
| 77 | 184 | 3094001 | BRSTNOT19 | 1494663T6 (PROSNON01), 2083139X11F1 (UTRSNOT08), 3094001H1 (BRSTNOT19) |
| 78 | 185 | 3110061 | BRSTTUT15 | 986428R6 (LVENNOT03), 1449222R1 (PLACNOT02), 3085841F6 (HEAONOT03), |
| | | | | 3110061F7 (BRSTTUT15), 3110061H1 (BRSTTUT15), 4308349T6 (BRAUNOT01), |
| | | | | 4637040F6 (MYEPTXT01) |
| 79 | 186 | 3146614 | PENCNOT06 | 638370R1 (BRSTNOT03), 1398786T1 (BRAITUT08), 1435622F1 (PANCNOT08), |
| | | | | 1720684F6 (BLADNOT06), 2194122F6 (THYRTUT03), 2459594H1 (THYRNOT08), |
| | | | | 3146614H1 (PENCNOT06), 3278069H1 (STOMFET02), 3357696F6 (PROSTUT16) |
| 80 | 187 | 3295381 | TLYJINT01 | 2222227F6 (LUNGNOT18), 3295381H1 (TLYJINT01), SZZZ00995R1, SZZZ00226R1, |
| | | | | SZZZ00209R1, SZZZ00347R1, SZZZ00451R1 |
| 81 | 188 | 3364774 | PROSBPT02 | 1339847F6 (COLNTUT03), 1415866F6 (BRAINOT12), 2458556T6 (ENDANOT01), |
| | | | | 2515467F6 (LIVRTUT04), 2523246H1 (BRAITUT21), 3095773H1 (CERVNOT03), |
| | | | | 3315208F6 (293TF2T01), 3364774H1 (PROSBPT02), 3697590H1 (SININOT05), |
| | | | | 4618612H1 (BRAYDIT01), g1422476 |
| 82 | 189 | 3397777 | UTRSNOT16 | 2906192F6 (THYMNOT05), 3046506F7 (HEAANOT01), 3046506X329D1 (HEAANOT01), |
| | | | | 3046506X331D1 (HEAANOT01), 3397777F7 (UTRSNOT16), 3397777H1 (UTRSNOT16), |
| | | | | 3846636H1 (DENDNOT01), 4569754H2 (PROSTUT21) |
| 83 | 190 | 3403046 | ESOGNOT03 | 2754425R6 (THP1AZS08), 3403046H1 (ESOGNOT03), 3844619F6 (DENDNOT01) |
| 84 | 191 | 3538506 | SEMVNOT04 | 483831H1 (HNT2RAT01), 1451166F1 (PENITUT01), 3187785H1 (THYMON04), |
| | | | | 3538506F6 (SEMVNOT04), 3538506H1 (SEMVNOT04), 3868721F6 (BMARNOT03), |
| | | | | 5108547F6 (PROSTUS19), 5163595H1 (ENDIUNT01), 5324664H1 (FIBPFEN06), |
| | | | | g2056736 |
| 85 | 192 | 3575519 | BRONNOT01 | 970343R6 (MUSCNOT02), 975169R6 (MUSCNOT02), 3575519H1 (BRONNOT01), |
| | | | | SCSA04735V1, SCSA03846V1 |
| 86 | 193 | 3598694 | FIBPNOT01 | 1330295F1 (PANCNOT07), 3332508T6 (BRAIFET01), 3520552H1 (LUNGNON03), |
| | | | | 3598694H1 (FIBPNOT01), 5203510H1 (STOMNOT08), 5506937H1 (BRADDIR01), |
| | | | | SCCA00526V1, SCCA04468V1, SCCA03065V1, SCCA02377V1, SCCA00888V1, |
| | | | | SCCA02832V1 |
| 87 | 194 | 3638819 | LUNGNOT30 | 837827X22 (PROSNOT07), 837827X23 (PROSNOT07), 3638819H1 (LUNGNOT30) |
| 88 | 195 | 3717139 | PENCNOT10 | 3717139H1 (PENCNOT10), g2106014, g2980871 |
| 89 | 196 | 3892962 | BRSTTUT16 | 594617R6 (BRAVUNT02), 837890X18 (PROSNOT07), 1961640R6 (BRSTNOT04), |
| | | | | 2330093H1 (COLNNOT11), 2726737F6 (OVARTUT05), 3892962H1 (BRSTTUT16) |
| 90 | 197 | 4153521 | MUSLTMT01 | 118141F1 (MUSCNOT01), 487811X24 (HNT2AGT01), 487811X26 (HNT2AGT01), |
| | | | | 868070R6 (BRAITUT03), 1832527T6 (BRAINON01), 2851743T6 (BRSTTUT13), |
| | | | | 4153521H1 (MUSLTMT01), 4531734H1 (PROSTMT03), SZZZ01004R1 |
| 91 | 198 | 4585038 | OVARNOT13 | 546958R6 (BEPINOT01), 656154H1 (EOSINOT03), 3415219H1 (PTHYNOT04), |
| | | | | 3683524H1 (HEAANOT01), 3750253H1 (UTRSNOT18), 4089875H1 (LIVRNOT06), |
| | | | | 4585038H1 (OVARNOT13), g756767, g756768 |
| 92 | 199 | 4674640 | NOSEDIT02 | 191268R1 (SYNORAB01), 1414304F6 (BRAINOT12), 3272067F6 (BRAINOT20), |
| | | | | 4674640H1 (NOSEDIT02), SCDA05786V1, SCDA07745V1, SZAP01877V1 |
| 93 | 200 | 4676066 | NOSEDIT02 | 875407R1 (LUNGAST01), 1478971F6 (CORPNOT02), 1749564F6 (STOMTUT02), |
| | | | | 2263128H1 (UTRSNOT02), 4676066H1 (NOSEDIT02), 5449856H1 (BSCNDIT02), |
| | | | | 5487675H1 (DRGTNON04), g3118452 |
| 94 | 201 | 4830687 | BRAVTXT03 | 534025F1 (BRAINOT03), 4830687H1 (BRAVTXT03) |
| 95 | 202 | 4880891 | UTRMTMT01 | 055751H1 (FIBRNOT01), 1288342F6 (BRAINOT11), 1288342T6 (BRAINOT11), |
| | | | | 1396095F6 (THYRNOT03), 1820602F6 (GBLATUT01), 2123331F6 (BRSTNOT07), |
| | | | | 2462011F6 (THYRNOT08), 2645166X303D1 (OVARTUT03), 2666343H1 (ADRETUT06), |
| | | | | 2666343T6 (ADRETUT06), 2715208F6 (THYRNOT09), 2881019F6 (UTRSTUT05), |
| | | | | 3448078X331D1 (UTRSNON03), 4880891H1 (UTRMTMT01), 5465061H1 (LNODNOT11), |
| | | | | 5503746H1 (BRABDIR01), SBLA03155F1, SBLA02267F1 |
| 96 | 203 | 4909754 | THYMDIT01 | 014580H1 (THP1PLB01), 1348640F6 (PROSNOT11), 1685157F6 (PROSNOT15), |
| | | | | 3427741H1 (BRSTNOR01), 3540578H1 (SEMVNOT04), 4909754F6 (THYMDIT01), |
| | | | | 4909754H1 (THYMDIT01), 5834707H1 (BRAIDIT05), g1940399 |
| 97 | 204 | 4911931 | THYMDIT01 | 428504F1 (BLANDNOT01), 468041R6 (LATRNOT01), 2342984F6 (TESTTUT02), |
| | | | | 2887138H1 (SINJNOT02), 4911931H1 (THYMDIT01) |
| 98 | 205 | 4920433 | TESTNOT11 | 2006765R6 (TESTNOT03), 4920433F6 (TESTNOT11), 4920433H1 (TESTNOT11) |
| 99 | 206 | 5042113 | COLHTUT01 | 537782R6 (LNODNOT02), 537782T6 (LNODNOT02), 724003H1 (SYNOOAT01), |
| | | | | 2700935X302B2 (OVARTUT10), 2700935X302F1 (OVARTUT10), 3572973T6 |
| | | | | (BRONNOT01), 5042113H1 (COLHTUT01), SBIA02608D1, SBIA08390D1 |
| 100 | 207 | 5083853 | LNOGTUT01 | 1537455H1 (SINTTUT01), 5083853F6 (LNOGTUT01), 5083853H1 (LNOGTUT01), |
| | | | | 5083853T6 (LNOGTUT01) |
| 101 | 208 | 5283981 | TESTNON04 | 542319F1 (OVARNOT02), 542319X15F1 (OVARNOT02), 542319X17F1 (OVARNOT02), |
| | | | | 1710519F6 (PROSNOT16), 5283981H1 (TESTNON04) |
| 102 | 209 | 5510549 | BRADDIR01 | 1257226F6 (MENITUT03), 1654887F6 (PROSTUT08), 1866033F6 (PROSNOT19), |
| | | | | 2309180H1 (NGANNOT01), 2516285F6 (LIVRTUT04), 3558606H1 (LUNGNOT31), |
| | | | | 4689374H1 (LIVRTUT11), 5510549H1 (BRADDIR01) |
| 103 | 210 | 5544862 | TESTNOC01 | 1210853R1 (BRSTNOT02), 1803417F6 (SINTNOT13), 5544862F6 (TESTNOC01), |
| | | | | 5544862H1 (TESTNOC01), 5544862T6 (TESTNOC01), 5547247F6 (TESTNOC01), |
| | | | | g989649, g3246546, g2112974, g697810 |
| 104 | 211 | 5573394 | TLYMNOT08 | 027981H1 (SPLNFET01), 310525T6 (TMLR2DT01), 826528R1 (PROSNOT06), |
| | | | | 868061R6 (BRAITUT03), 1985188T6 (LUNGAST01), 2207165F6 (SINTFET03), |
| | | | | 5507004H1 (BRADDIR01), 5573394H1 (TLYMNOT08), SBIA11388D1, SBIA11986D1, |
| | | | | SBIA03475D1 |
| 105 | 212 | 5850840 | FIBAUNT02 | 232422F1 (SINTNOT02), 232442R1 (SINTNOT02), 826837R1 (PROSNOT06), |
| | | | | 1286853F1 (BRAINOT11), 2058494R6 (OVARNOT03), 2842471F6 (DRGLNOT01), |
| | | | | 3105825F6 (BRSTTUT15), 3617707H1 (EPIPNOT01), 3620903H1 (BRSTNOT25), |
| | | | | 4148432H1 (SINITUT04), 5850840H1 (FIBAUNT02) |
| 106 | 213 | 5942936 | COLADIT05 | 121785R6 (MUSCNOT01), 797379T6 (OVARNOT03), 797379X14R1 (OVARNOT03), |
| | | | | 797379X25R1 (OVARNOT03), 3690756H1 (HEAANOT01), 5942936H1 (COLADIT05) |
| 107 | 214 | 5951431 | LIVRTUN04 | 623984R6 (PGANNOT01), 676513T6 (CRBLNOT01), 1730442F6 (BRSTTUT08), |
| | | | | 2640175F6 (LUNGTUT08), 3360767F6 (PROSTUT16), 5951431H1 (LIVRTUN04), |
| | | | | SAEA03186R1 |

TABLE 2

| Polypep- | | | Potential | Potential | | | |
|---|---|---|---|---|---|---|---|
| tide | | Amino | Phospho- | Glycosyla- | | | Analytical |
| SEQ ID | | Acid | rylation | tion | Signature Sequences, | | Methods and |
| NO: | | Residues | Sites | Sites | Motifs and Domains | Homologous Sequences | Databases |
| 1 | 095210 | 463 | S72 T7 S16 S49 | N38 N53 | ATP/GTP-binding site | g498721 | MOTIFS |
| | | | T371 T58 S68 | | motif A (P-loop): | zinc finger protein | BLAST_GENBANK |
| | | | S72 | | G412-S419 | [Homo sapiens] | BLAST_PFAM |
| | | | | | Zinc finger C2H2 type | Abrink, M. et al. | BLIMPS_BLOCKS |
| | | | | | domain: C133-H153 | (1995) DNA Cell Biol. | BLIMPS_PRODOM |
| | | | | | C161-H181 C189-H209 | 14: 125-136 | BLAST_DOMO |
| | | | | | C217-H237 C245-H265 | | |
| | | | | | C273-H293 C301-H321 | | |
| | | | | | C329-H349 C357-H377 | | |
| | | | | | C385-H405 C413-H423 | | |
| | | | | | C441-H461 | | |
| | | | | | KRAB box domain: V6- | | |
| | | | | | R66 | | |
| 2 | 157953 | 216 | T28 T140 T2 | N152 | bZIP transcription | g4996451 leucine- | MOTIFS |
| | | | T139 S210 | | factors basic domain | zipper protein | BLAST_GENBANK |
| | | | | | signature: K147-R163 | | BLAST_PFAM |
| | | | | | | | BLIMPS_BLOCKS |
| | | | | | | | BLAST_DOMO |
| 3 | 159196 | 284 | S153 S44 T189 | N94 N95 | Zinc finger C2H2 type | g55471 Zinc finger | MOTIFS |
| | | | T232 S3 T62 | N207 | domain: C86-H106 C114- | protein expressed in | BLAST_GENBANK |
| | | | S125 S148 T245 | | H134 C142-H162 C170- | post-meiotic | BLAST_PFAM |
| | | | Y140 Y196 | | H190 C198-H218 C226- | spermatogenesis | |
| | | | | | H246 C254-H274 | Denny, P. and | |
| | | | | | | Ashworth, A. (1991) | |
| | | | | | | Gene 106: 221-227 | |
| 4 | 343338 | 1416 | S817 T406 S142 | N40 N261 | ATP/GTP-binding site | g7717364 | MOTIFS |
| | | | S236 S272 S329 | N409 | motif A (P-loop): | homolog to cAMP | BLAST_GENBANK |
| | | | S395 S412 S413 | N467 | A1086-T1093 A1131- | response element | BLAST_PFAM |
| | | | T426 S427 S439 | N1040 | T1138 | binding and beta | PROFILESCAN |
| | | | S474 S475 S476 | N1130 | Beta-transducin family | transducin family | BLIMPS_PRINTS |
| | | | T531 S669 S711 | N1167 | Trp-Asp repeats | proteins [Homo | BLAST_PRODOM |
| | | | T735 T832 S876 | | signature: v34-S48 | sapiens] | BLAST_DOMO |
| | | | S878 T954 T960 | | L77-L91 | | |
| | | | S972 S1051 | | Bromodomain signature: | | |
| | | | T1138 S1378 T42 | | A778-H853, P935-T991 | | |
| | | | S141 T262 T307 | | | | |
| | | | S315 T336 T345 | | | | |
| | | | S381 T400 T469 | | | | |
| | | | S482 S506 T625 | | | | |
| | | | T634 T707 T803 | | | | |
| | | | S843 S869 S891 | | | | |
| | | | T892 S993 S1002 | | | | |
| | | | T1033 S1103 T31 | | | | |
| | | | S1143 S1169 | | | | |
| | | | T1317 S1329 | | | | |
| | | | S1336 S1397 | | | | |
| | | | Y658 T1406 Y346 | | | | |
| | | | Y813 Y945 Y970 | | | | |
| 5 | 402386 | 426 | S292 T14 S65 | N12 | Zinc finger C2H2 type | g487785 zinc finger | MOTIFS |
| | | | S115 S24 T36 | | domain: F6-G44, C102- | protein ZNF136 | BLAST_GENBANK |
| | | | T139 T164 T192 | | H124, Y169-H191, C171- | Tommerup, N. and | BLAST_PFAM |
| | | | S196 S380 Y229 | | H191, Y225-H247, Y253- | Vissing, H. (1995) | BLIMPS_PRODOM |
| | | | | | H276, H282-H304, Y310- | Genomics 27: 259-264 | BLAST_PRODOM |
| | | | | | H332, Y338-H360, Y366- | | BLAST_DOMO |
| | | | | | H388 | | |
| | | | | | KRAB box domain: V4- | | |
| | | | | | V67 | | |
| 6 | 456487 | 686 | S407 T408 S27 | N79 N128 | Putative GTPase | g3880859 similar to | MOTIFS |
| | | | S94 S117 T176 | N213 | activating protein for | Ank repeat (2 domains) | BLAST_GENBANK |
| | | | S185 T224 S225 | N616 | Arf: A464-E584 | | BLAST_PFAM |
| | | | S260 S318 T426 | | HIV REV interacting | | BLIMPS_PRINTS |
| | | | T427 S460 S542 | | protein: N476-R512, | | BLAST_PRODOM |
| | | | S558 T559 S569 | | V516-N537 | | BLAST_DOMO |
| | | | T595 S611 T618 | | Zinc finger motif: | | |
| | | | S668 T6 T135 | | Q468-P581 | | |
| | | | S247 S256 S278 | | | | |
| | | | S293 T299 T337 | | | | |
| | | | S357 S386 S451 | | | | |
| | | | S555 | | | | |
| 7 | 490256 | 348 | T3 T108 T114 | | Zinc finger C2H2 type | g2316003 | MOTIFS |
| | | | T163 T181 S29 | | domain: C238-H258 | zinc finger protein | BLAST_GENBANK |
| | | | S134 S302 | | C266-H286 C294-H314 | [Homo sapiens] | BLAST_PFAM |
| | | | | | C322-H342 | Lee, P. L. et al. | BLIMPS_BLOCKS |
| | | | | | Zinc finger motif: E8- | (1997) Genomics | BLIMPS_PRINTS |
| | | | | | Q173 | 43: 191-201 | BLIMPS_PRODOM |
| | | | | | | | BLAST_DOMO |
| 8 | 494740 | 181 | T22 T37 S60 T78 | | Zinc finger motif: | g487836 transcription | MOTIFS |
| | | | T87 S12 T70 | | F79-G117 | factor | BLAST_GENBANK |
| | | | T124 S157 | | KRAB box: V77-R126 | | BLAST_PFAM |
| | | | | | | | BLIMPS_PRODOM |
| | | | | | | | BLAST_PRODOM |
| | | | | | | | BLAST_DOMO |
| 9 | 507475 | 126 | S2 S15 S71 S104 | | TFIIS zinc ribbon | g7212805 | MOTIFS |
| | | | y97 | | domain signature: G65- | transcription- | BLAST_GENBANK |
| | | | | | K123 | associated zinc ribbon | PROFILESCAN |
| | | | | | | protein [Homo sapiens] | BLIMPS_BLOCKS |
| | | | | | | Fan, W. et al. (2000) | BLAST_DOMO |
| | | | | | | Genomics 63: 139-141 | |
| 10 | 531581 | 610 | S177 S410 T438 | N194 | Zinc finger C2H2 type | g8843908 | MOTIFS |
| | | | T466 S44 S55 | N206 | domain: C304-H324 | zinc finger protein | BLAST_GENBANK |
| | | | S125 T146 S233 | | C332-H352 C360-H381 | SBBIZ1 [Homo sapiens] | HMMER_PFAM |
| | | | S239 S282 S289 | | C389-H409 C417-H437 | | BLIMPS_BLOCKS |
| | | | S482 S507 S523 | | C445-H465 C473-H493 | | BLIMPS_PRINTS |
| | | | S531 S532 T537 | | C501-H522 | | BLIMPS_PRODOM |
| | | | S539 T179 S188 | | Zinc finger activator | | BLAST_PRODOM |
| | | | T255 S279 S316 | | domain: M9-E124 | | BLAST_DOMO |
| | | | S462 Y81 Y415 | | | | |
| | | | Y443 Y471 | | | | |
| 11 | 675190 | 111 | T102 S17 S24 | N3 | Zinc finger protein | g10442700 | MOTIFS |
| | | | T33 T67 S9 S43 | | domain: F25-G63 | zinc-finger protein | BLAST_GENBANK |
| | | | S97 | | KRAB box domain: S22- | ZBRK1 [Homo sapiens] | BLIMPS_PRODOM |
| | | | | | P94 | Zheng L. et al. (2000) | BLAST_DOMO |
| | | | | | | Mol Cell 6: 757-768 | BLAST_PFAM |
| 12 | 685434 | 152 | T6 T17 S109 | N15 | | g4336830 RFX-Bdelta4 | MOTIFS |
| | | | | | | immunodeficiency- | BLAST_GENBANK |
| | | | | | | associated | |
| | | | | | | transcription factor | |
| | | | | | | Nagarajan, U. M. et al. | |
| | | | | | | (1999) Immunity | |
| | | | | | | 10: 153-162 | |
| 13 | 788663 | 131 | S30 S65 T73 | N122 | Transcription factor | G2583171 CCAAT-binding | MOTIFS |
| | | | S124 T45 S60 | | domain: R15-K96 | transcription factor | BLAST_GENBANK |
| | | | S65 | | | subunit AAB-1 | BLAST_DOMO |
| | | | | | | Chen, H. et al. (1998) | |
| | | | | | | Genetics 148: 123-130 | |
| 14 | 870100 | 541 | S7 S24 S69 S85 | N192 | Zinc finger C2H2 type | g189044 zinc finger | MOTIFS |
| | | | S99 S253 T255 | N450 | domain: C152-H172, | protein 42 (MZF-1, | BLAST_GENBANK |
| | | | T302 S505 S151 | N454 | C180-H200, C208-H228, | preferentially | HMMER_PFAM |
| | | | T245 T315 S356 | | C362-H382, C390-H410, | expressed in myeloma | BLIMPS_BLOCKS |
| | | | S521 | | C418-H438, C446-H466, | cells) | BLIMPS_PRINTS |
| | | | | | C474-H494 | Hromas, R. et al. | BLIMPS_PRODOM |
| | | | | | | (1991) J Biol Chem | |
| | | | | | | 266: 14183-14187 | |
| 15 | 879500 | 1828 | S1194 S1283 | N174 | Helicases conserved c- | g5106572 | MOTIFS |
| | | | S1307 S1390 | N725 | terminal domain: D672- | transcriptional | BLAST_GENBANK |
| | | | S1395 S1467 | N794 | G755 | activator SRCAP | HMMER_PFAM |
| | | | S1530 S1554 | N1197 | | Johnston, H. et al. | BLAST_PRODOM |
| | | | S1614 S1629 | | | (1999) J Biol Chem | BLAST_DOMO |
| | | | S1651 S1652 | | | 274: 16370-16376 | |
| | | | S1653 S1717 | | | | |
| | | | S1766 S1770 | | | | |
| | | | S1775 S1820 | | | | |
| | | | S323 S443 S487 | | | | |
| | | | S497 S691 S716 | | | | |
| | | | S767 S822 S894 | | | | |
| | | | S921 S926 S980 | | | | |
| | | | S994 T1271 | | | | |
| | | | T1322 T1333 | | | | |
| | | | T1354 T1482 | | | | |
| | | | T1712 T1731 | | | | |
| | | | T1784 T322 T451 | | | | |
| | | | T549 T692 T727 | | | | |
| | | | T770 T803 T908 | | | | |
| | | | T976 | | | | |
| 16 | 975377 | 482 | S185 S200 S258 | N306 | Zinc finger C3HC4 type | g1304599 ZNF127-Xp | MOTIFS |
| | | | S295 S319 S330 | | signature: K57-L112, | (associated with | BLAST_GENBANK |
| | | | S366 S408 S463 | | I208-C236, C305-I314 | Prader-Willi | BLAST_PFAM |
| | | | T118 T123 T196 | | | behavioral syndrome) | BLIMPS_BLOCKS |
| | | | T205 T209 T461 | | | Jong, M. T. et al. | |
| | | | T60 Y230 Y77 | | | (1999) Hum Mol Genet | |
| | | | | | | 8: 783-793 | |
| 17 | 1208721 | 264 | S11 S59 T100 | | | | MOTIFS |
| | | | T114 T235 S259 | | | | |
| | | | S23 S138 | | | | |
| 18 | 1234329 | 350 | S170 S229 T290 | | Zinc finger C3HC4 type | g3880441 | MOTIFS |
| | | | S303 S129 S235 | | signature: C298-C338 | similar to zinc finger | BLAST_GENBANK |
| | | | T331 | | PHD-finger: R313-Q327 | C3HC4 type | HMMER_PFAM |
| | | | | | | | PROFILESCAN |
| | | | | | | | BLIMPS_PFAM |
| 19 | 1238747 | 549 | S102 S175 S248 | N77 N328 | SAND DNA-binding | g9964115 | MOTIFS |
| | | | S273 S296 S303 | | domain: S454-L535 | transcriptional | BLAST_GENBANK |
| | | | S329 S346 S364 | | | coactivator Sp110 | HMMER_PFAM |
| | | | S437 S438 S485 | | | [Homo sapiens] | |
| | | | T201 T271 T287 | | | Bloch, D. B. et al. | |
| | | | T370 T375 T396 | | | (2000) Mol Cell Biol | |
| | | | T44 T467 T498 | | | 20: 6138-6146 | |
| | | | T524 T70 | | | | |
| 20 | 1265980 | 337 | S22 T84 T85 T56 | | Helix-loop-helix DNA | g4566748 basic helix- | MOTIFS |
| | | | S131 S238 S242 | | binding domain: R95- | loop-helix | BLAST_GENBANK |
| | | | T247 T326 S47 | | S147, M1-L73 | transcription factor | HMMER_PFAM |
| | | | T56 T127 T135 | | Myc-type ‘helix-loop- | Ndr1 | PROFILESCAN |
| | | | S230 S272 Y281 | | helix’ dimerization | Liao, J. et al. (1999) | BLIMPS_BLOCKS |
| | | | | | domain signature: | DNA Cell | BLAST_PRODOM |
| | | | | | E103-R118, T127-S147, | Biol 18: 333-344 | BLAST_DOMO |
| | | | | | N111-N164, E66-Q171 | | |
| | | | | | Transcription | | |
| | | | | | regulation domain: | | |
| | | | | | R191-N337 | | |
| 21 | 1297333 | 581 | S16 S29 T41 S47 | N78 N90 | Zinc finger C2H2 type | g387079 zinc finger | MOTIFS |
| | | | T35 S92 S110 | N201 | domain: C135-H155 | protein (mkr5) | BLAST_GENBANK |
| | | | T184 S254 T368 | N426 | C163-H183 C191-H212, | Chowdhury, K. et al. | HMMER_PFAM |
| | | | S480 S493 S531 | | C220-H240, C248-H268, | (1988) Nucleic Acids | BLIMPS_BLOCKS |
| | | | Y56 Y89 | | C276-H296 C304-H324 | Res 16: 9995-10011 | BLIMPS_PRINTS |
| | | | | | C332-H352 C360-H380, | | BLIMPS_PRODOM |
| | | | | | C388-H408, C416-H436, | | BLAST_PRODOM |
| | | | | | C444-H464, C472-H492, | | BLAST_DOMO |
| | | | | | C500-H520 | | |
| 22 | 1312824 | 591 | S126 S127 S167 | N296 | Ets-related | g972940 Elf-1 | MOTIFS |
| | | | S278 S293 S389 | N384 | transcription factor | Transcription | BLAST_GENBANK |
| | | | S404 S435 S460 | N489 | domain: D273-F591, | regulation protein | HMMER_PFAM |
| | | | S510 S546 S64 | | I180-F261 I180-K193, | Davis, J. N. and | PROFILESCAN |
| | | | S88 T203 T298 | | E206-K224, H225-Y243, | Roussel, M. F. (1996) | BLIMPS_BLOCKS |
| | | | T377 T554 Y233 | | Y244-K262 | Gene 171: 265-269 | BLIMPS_PRINTS |
| | | | | | | | BLAST_PRODOM |
| | | | | | | | BLAST_DOMO |
| 23 | 1359294 | 767 | S141 S391 S43 | N18 | ‘Cold-shock’ DNA- | g57455 unr protein | MOTIFS |
| | | | S461 S463 S485 | N288 | binding domain | Ferrer, N. et al. | BLAST_GENBANK |
| | | | S567 S620 S664 | N549 | signature: Y37-V88, | (1999) DNA Cell Biol | HMMER_PFAM |
| | | | S75 T111 T201 | N728 | L121-M146, F166-R215 | 18: 209-218 | BLIMPS_BLOCKS |
| | | | T278 T291 T301 | | F329-V365, F499-N549, | | BLAST_PRODOM |
| | | | T494 T580 T646 | | F654-W705 | | BLAST_DOMO |
| | | | T696 T730 T79 | | Unr protein DNA | | |
| | | | | | binding repeat domain: | | |
| | | | | | E98-D767 | | |
| 24 | 1377380 | 206 | S11 S131 S15 | | | g7012714 | MOTIFS |
| | | | S152 S163 S167 | | | L2DTL WD-40 repeat | BLAST_GENBANK |
| | | | S181 S193 S2 | | | protein [Homo sapiens] | |
| | | | S34 S38 S84 S85 | | | | |
| | | | S93 T51 | | | | |
| 25 | 1383473 | 352 | S74 T95 T154 | N104 | Signal peptide motif: | g4587558 Similar to X- | MOTIFS |
| | | | S165 S222 S322 | N205 | M1-A23 | linked apoptosis | BLAST_GENBANK |
| | | | S207 S236 S297 | | Transmembrane motif: | inhibitor | HMMER |
| | | | S308 | | L243-L259, C302-C339 | | |
| | | | | | Baculovirus inhibitor | | |
| | | | | | of apoptosis protein | | |
| | | | | | repeat (BIR): L298- | | |
| | | | | | C336 | | |
| 26 | 1388860 | 532 | S153 S27 S409 | N42 N65 | Zinc finger C2H2 type | g4519270 Kruppel-type | MOTIFS |
| | | | S465 S520 T103 | | domain: C201-H221 | zinc finger protein | BLAST_GENBANK |
| | | | T17 T360 T39 | | C229-H249 C257-H277 | Katoh, O. (1998) | HMMER_PFAM |
| | | | T49 Y138 Y367 | | C285-H305 C313-H333 | Biochem. Biophys. Res. | BLIMPS_BLOCKS |
| | | | | | C341-H361 C369-H389 | Commun. 249: 595-600 | BLIMPS_PRINTS |
| | | | | | C397-H417 C425-H445, | | BLIMPS_PRODOM |
| | | | | | C453-H473, C481-H500, | | BLAST_PRODOM |
| | | | | | C508-H528 | | BLAST_DOMO |
| | | | | | Zinc finger domain: | | |
| | | | | | F9-G47, K48-K146 | | |
| | | | | | KRAB box domain: D5- | | |
| | | | | | E78 | | |
| 27 | 1395322 | 444 | S105 S134 S155 | N54 | Zinc finger C2H2 type | g6063139 | MOTIFS |
| | | | S319 S375 T110 | N153 | domain: C283-H303, | POZ/zinc finger | BLAST_GENBANK |
| | | | T291 T347 T378 | N166 | C311-H331, C339-H359 | transcription factor | HMMER_PFAM |
| | | | T69 T7 T88 Y40 | N287 | C367-H387 C420-H440 | ODA-8 [Mus musculus] | BLIMPS_BLOCKS |
| | | | | | | | BLIMPS_PRINTS |
| | | | | | | | BLIMPS_PRODOM |
| 28 | 1419370 | 347 | S183 T307 T14 | | Zinc finger C3HC4 type | g11611473 | MOTIFS |
| | | | T263 T300 | | signature: C164-C202 | Deltex3 | BLAST_GENBANK |
| | | | | | | Kishi, N. et al. | HMMER_PFAM |
| | | | | | | (2001) Int. J. Dev. | BLIMPS_BLOCKS |
| | | | | | | Neurosci. 19: 21-35 | PROFILESCAN |
| 29 | 1429773 | 308 | S29 S31 T250 | N112 | Transmembrane domain: | g7542723 | MOTIFS |
| | | | S257 Y130 | | P213-M237 | DHHC1 protein [Homo | BLAST_GENBANK |
| | | | | | | sapiens] | HMMER |
| 30 | 1470820 | 80 | S14 S32 T21 S36 | | GC-rich sequence DNA | | MOTIFS |
| | | | S72 Y63 | | binding factor domain: | | |
| | | | | | R11-V75 (P-value = 5.9 × 10⁻⁶ | | |
| 31 | 1483455 | 570 | S116 S132 S181 | N212 | ATP/GTP-binding site | g7688669 | MOTIFS |
| | | | S211 S470 S564 | N502 | motif A (P-loop) A216- | zinc finger protein | BLAST_GENBANK |
| | | | S70 S79 S87 T14 | N530 | S223 | ZNF140-like protein | HMMER_PFAM |
| | | | T143 T168 T237 | | Zinc finger C2H2 type | [Homo sapiens] | BLIMPS_BLOCKS |
| | | | T5 T54 T569 T88 | | domain C238-H258, | | BLIMPS_PRINTS |
| | | | | | C266-H286, C294-H314, | | BLIMPS_PRODOM |
| | | | | | C322-H342, C350-H370, | | BLAST_PRODOM |
| | | | | | C378-H398, C406-H426, | | BLAST_DOMO |
| | | | | | C434-H454, C462-H482, | | |
| | | | | | C490-H510, C518-H538 | | |
| | | | | | Zinc finger protein | | |
| | | | | | motif: V4-W77 | | |
| | | | | | KRAB box domain: V4- | | |
| | | | | | M73 | | |
| 32 | 1527064 | 390 | S107 S145 S167 | N160 | Transcription factor | g 532313 NF45 protein | MOTIFS |
| | | | S344 S354 S52 | N214 | domain: V102-E371 | Kao, P. N. et al. | BLAST_GENBANK |
| | | | T112 T162 T172 | | Heat shock factor | (1994) J Biol Chem | BLIMPS_PRINTS |
| | | | T219 T352 | | (transcriptional | Aug 12, 1994; | BLAST_DOMO |
| | | | | | activator) signature: | 269: 20691-9 | |
| | | | | | L317-I329 | | |
| 33 | 1557491 | 601 | S158 S163 S179 | N2 N104 | Zinc finger C2H2 type | g 220643 zinc finger | MOTIFS |
| | | | S219 S313 S334 | N484 | domain C418-H438, | protein | BLAST_GENBANK |
| | | | S355 S513 S559 | | C446-H466, C474-H494, | | HMMER_PFAM |
| | | | S82 T186 T187 | | C502-H522, C533-H553 | | BLIMPS_BLOCKS |
| | | | T190 T218 T246 | | | | BLIMPS_PRINTS |
| | | | T318 T412 T430 | | | | BLIMPS_PRODOM |
| | | | T482 T486 T514 | | | | |
| | | | T594 Y75 | | | | |
| 34 | 1576862 | 834 | S127 S135 S36 | | PHD finger: C219-I233 | g1510153 similar to | MOTIFS |
| | | | S50 S520 S531 | | Zinc finger protein | human bromodomain | BLAST_GENBANK |
| | | | S628 S679 S696 | | motif: C202-R260, | protein BR140 | BLIMPS_PFAM |
| | | | S70 S745 S798 | | L256-H364 | Nagase, T. et al. DNA | BLAST_PRODOM |
| | | | S803 S805 S9 | | Peregrin | Res 1996 Oct | BLAST_DOMO |
| | | | T391 T445 T487 | | transcriptional | 31; 3(5): 321-9, 341-54 | |
| | | | T543 T663 T688 | | regulator domain: | | |
| | | | T724 T761 T791 | | D199-K389, A524-A551 | | |
| 35 | 1609731 | 499 | S104 S108 S16 | N139 | Zinc finger C2H2 type | g456269 zinc finger | MOTIFS |
| | | | S50 S56 S81 | | domain: C169-H189, | protein 30 | BLAST_GENBANK |
| | | | T155 T259 T7 | | C197-H217, C225-H245, | Denny, P. and | HMMER_PFAM |
| | | | Y134 | | C253-H273, C281-H301, | Ashworth, A. (1994) | BLIMPS_BLOCKS |
| | | | | | C309-H329, C337-H358, | Mamm. Genome | BLIMPS_PRINTS |
| | | | | | C365-H385, C393-H413, | 5: 643-645 | BLIMPS_PRODOM |
| | | | | | C421-H441, C449-H469, | | BLAST_PRODOM |
| | | | | | C477-H497 | | BLAST_DOMO |
| | | | | | KRAB box domain: Q3-V71 | | |
| 36 | 1674538 | 402 | S102 S158 S193 | N303 | Zinc finger C2H2 type | g 55473 zinc finger | MOTIFS |
| | | | S219 S324 S384 | N382 | domain: C73-H93, C101- | protein | BLAST_GENBANK |
| | | | T12 T292 T3 | | H121, C129-H149, C157- | | HMMER_PFAM |
| | | | T344 T354 Y351 | | H177, C185-H205, C213- | | BLIMPS_BLOCKS |
| | | | | | H233, C241-H261, C269- | | BLAST_PRODOM |
| | | | | | H289 | | BLAST_DOMO |
| | | | | | Zinc finger protein | | |
| | | | | | domain: | | |
| | | | | | E62-H121, Q82-K153, | | |
| | | | | | K162-K237, K246-K319 | | |
| 37 | 1675287 | 579 | S134 S22 S270 | | Zinc finger C3HC4 type | g1136384 C3HC4 | MOTIFS |
| | | | S347 S57 T176 | | signature: C400-C408 | containing protein | BLAST_GENBANK |
| | | | T222 T293 T526 | | Zinc finger protein | | BLIMPS_BLOCKS |
| | | | T535 T537 T82 | | domain: C206-C408 | | BLAST_PRODOM |
| | | | Y285 Y362 | | | | |
| 38 | 1693903 | 426 | S12 S231 S290 | N246 | CCCH-Zinc finger | | MOTIFS |
| | | | S328 S360 S381 | | protein motif: | | BLIMPS-PFAM |
| | | | S63 T114 T318 | | C113-H123 | | |
| | | | T408 | | | | |
| 39 | 1702962 | 266 | S78 T127 T163 | N20 | Zinc finger C2H2 type | g5001720 | MOTIFS |
| | | | T171 T196 T261 | | domain: F175-H197, | odd-skipped related 1 | BLAST_GENBANK |
| | | | Y203 | | H193-C205, E194-H221, | protein [Mus musculus] | BLAST_PFAM |
| | | | | | C205-H225, H225-H249, | So, P. L. and | BLIMPS_BLOCKS |
| | | | | | P230-S243, F231-H253 | Danielian P. S. (1999) | BLIMPS_PRINTS |
| | | | | | | Mech. Dev. 84: 157-160 | BLIMPS_PRODOM |
| | | | | | | | BLAST_DOMO |
| 40 | 1712916 | 358 | S160 S164 S21 | N228 | ‘Homeobox’ domain | g 1899230 iroquois- | MOTIFS |
| | | | S230 S255 S267 | N238 | signature: | class homeodomain | BLAST_GENBANK |
| | | | S269 S286 S291 | N249 | K74-K129, N95-L106, | protein IRX-2a | BLIMPS_BLOCKS |
| | | | S299 S350 T101 | N284 | L106-K129, S110-K129 | | BLIMPS_PRINTS |
| | | | T131 T99 Y97 | | | | BLAST_DOMO |
| 41 | 1748313 | 260 | S102 S183 S204 | N53 | | | MOTIFS |
| | | | S228 S35 S74 | N124 | | | |
| | | | T13 T145 T167 | N178 | | | |
| | | | T176 T30 | | | | |
| 42 | 1754833 | 263 | S109 S177 S45 | N258 | Zinc finger C3HC4 type | g3790583 RING-H2 | MOTIFS |
| | | | S83 S95 T31 T55 | | signature: C181-C221, | finger protein RHC1a | BLAST_GENBANK |
| | | | T59 T67 T74 | | S177-T232 | | HMMER_PFAM |
| | | | | | | | PROFILESCAN |
| 43 | 1798701 | 581 | S356 S368 S43 | N77 | | g6688742 | MOTIFS |
| | | | S473 S8 T145 | N164 | | putative TH1 protein | BLAST_GENBANK |
| | | | T166 T202 T309 | N550 | | [Mus musculus] | |
| | | | T360 T377 T425 | | | | |
| | | | T486 T556 T559 | | | | |
| | | | T56 T95 Y175 | | | | |
| 44 | 1842496 | 117 | S4 | | | g 4336506 | MOTIFS |
| | | | | | | transcription | BLAST_GENBANK |
| | | | | | | elongation factor | |
| 45 | 1868613 | 202 | | | | g171091 | MOTIFS |
| | | | | | | ASF1 [Saccharomyces | BLAST_GENBANK |
| | | | | | | cerevisiae] DNA | BLAST_PRODOM |
| | | | | | | repair-associated | |
| | | | | | | protein | |
| | | | | | | Le, S. et al. (1997) | |
| | | | | | | Yeast 13: 1029-1042 | |
| 46 | 1870609 | 442 | S166 S18 S308 | N398 | Zinc finger C3HC4 type | g5931953 | MOTIFS |
| | | | S315 S322 S341 | | signature: C140-C177 | autocrine motility | BLAST_GENBANK |
| | | | S358 S373 S400 | | | factor receptor [Mus | BLAST_PFAM |
| | | | S401 T129 T176 | | | musculus] | |
| | | | T26 T303 T333 | | | Shimizu, K. et al. | |
| | | | T422 | | | (1999) FEBS Lett. | |
| | | | | | | 456: 295-300 | |
| 47 | 1871961 | 765 | S177 S198 S264 | N81 | Zinc finger C2H2 type | g6672074 | MOTIFS |
| | | | S514 S547 S604 | N175 | domain: C595-H617, | nuclear protein NP94 | BLAST_GENBANK |
| | | | S682 T225 T269 | N520 | C687-H709 | [Homo sapiens] | |
| | | | T349 T504 T645 | | GAL 11 transcription | | |
| | | | T696 | | factor motif: T347- | | |
| | | | | | M627 | | |
| | | | | | Coiled coil domain: | | |
| | | | | | Q4-Q44, E206-Q428 | | |
| 48 | 1876258 | 352 | S118 S153 S222 | | ATP/GTP-binding site | g 4165083 growth | MOTIFS |
| | | | S255 S317 S9 | | motif A (P-loop): | factor independence-1B | BLAST_GENBANK |
| | | | T250 T289 T321 | | A193-T200 | (transcription factor | HMMER_PFAM |
| | | | | | Zinc finger C2H2 type | expressed in t- | BLIMPS_BLOCKS |
| | | | | | domain: C165-H186, | lymphocytes) | BLIMPS_PRINTS |
| | | | | | C168-S222, C194-H214, | B. Rodel et al. | BLIMPS_PRODOM |
| | | | | | C244-H264, C247-Q301, | Genomics 1998 Dec | BLAST_PRODOM |
| | | | | | C272-H292, C275-E329, | 15; 54(3): 580-2 | BLAST_DOMO |
| | | | | | P297-S310, C300-H320, | | |
| | | | | | L313-G322, H316-C328, | | |
| | | | | | C328-H349, F323-K352 | | |
| 49 | 1929822 | 1102 | S1001 S1008 | N50 | Homeobox domain: L771- | g4406073 activity- | MOTIFS |
| | | | S1051 S1067 S11 | N132 | R813 | dependent | BLAST_GENBANK |
| | | | S346 S365 S425 | N315 | Zinc finger C2H2 type | neuroprotective | BLIMPS_BLOCKS |
| | | | S740 S805 S82 | N398 | domain: C514-H536 | protein (contains a | |
| | | | S874 S891 S921 | N439 | Glutaredoxin active | glutaredoxin active | |
| | | | S934 S953 S955 | N486 | site: C514-V524 | site) | |
| | | | S970 S982 T142 | N674 | | Bassan, M. J Neurochem | |
| | | | T171 T18 T443 | N857 | | 1999 Mar; 72(3): 1283-93 | |
| | | | T488 T51 T52 | N887 | | | |
| | | | T520 T661 T782 | N951 | | | |
| | | | T995 Y764 Y818 | N1030 | | | |
| | | | | N1049 | | | |
| | | | | N1066 | | | |
| | | | | N1079 | | | |
| 50 | 1970095 | 121 | T25 S32 T39 T63 | N26 | | g5713279 | MOTIFS |
| | | | S72 S91 | | | Yippee protein | BLAST_GENBANK |
| | | | | | | [Drosophila | |
| | | | | | | melanogaster] | |
| 51 | 1975473 | 233 | T34 S8 S25 S65 | N26 | Signal peptide: M1- | g4704419 | MOTIFS |
| | | | T174 S199 | | A62 | WS basic-helix-loop- | BLAST_GENBANK |
| | | | | | Myc-type ‘helix-loop- | helix leucine zipper | HMMER_PFAM |
| | | | | | helix’ dimerization | protein [Homo sapiens] | BLIMPS_BLOCKS |
| | | | | | domain signature: L7- | Meng, X. et al. (1998) | BLAST_DOMO |
| | | | | | T63, V11-P122, R31- | Human Genetics | |
| | | | | | Q85, E39-H54, S65-Q85 | 103: 590-599 | |
| | | | | | FOS-type leucine | | |
| | | | | | zipper: L84-L105 | | |
| 52 | 1976527 | 147 | T63 S71 T114 | N28 N65 | Signal peptide: M1- | g9623363 | MOTIFS |
| | | | S122 | | A52 | DNA polymerase epsilon | BLAST_GENBANK |
| | | | | | NFYB transcription | p17 subunit [Homo | PROFILESCAN |
| | | | | | factor subunit: | sapiens] | BLIMPS_BLOCKS |
| | | | | | R4-K100, P5-R94, K20- | Li, Y. et al. (2000) | BLIMPS_PRINTS |
| | | | | | M76 | J. Biol. Chem. | BLAST_PRODOM |
| | | | | | CCAAT-binding | 275: 23247-23252 | BLAST_DOMO |
| | | | | | transcription factor | | |
| | | | | | motif: A56-R93, E6- | | |
| | | | | | D111 | | |
| 53 | 2108023 | 96 | T32 T7 S13 T50 | N36 | | g9294739 | MOTIFS |
| | | | T56 S73 | | | bithoraxoid-like | BLAST_GENBANK |
| | | | | | | protein [Homo sapiens] | |
| 54 | 2135746 | 259 | S56 S120 S166 | | Signal peptide: M1-G20 | | MOTIFS |
| | | | S181 S233 S23 | | | | SPSCAN |
| | | | S29 S89 T208 | | | | |
| 55 | 2154810 | 474 | S88 S156 S50 | N38 N97 | Zinc finger C2H2 type | g456269 zinc finger | MOTIFS |
| | | | S56 T80 T84 | | domain: C172-H192, | protein 30 | BLAST_GENBANK |
| | | | T124 S140 S145 | | C200-H220, C228-H248, | | HMMER_PFAM |
| | | | Y94 | | C256-H276, C284-H304, | | BLIMPS_BLOCKS |
| | | | | | C312-H332, C340-H360, | | BLIMPS_PRINTS |
| | | | | | C368-H388, C396-H416 | | BLIMPS_PRODOM |
| | | | | | Zinc finger protein | | BLAST_PRODOM |
| | | | | | motif: F8-G46 | | BLAST_DOMO |
| | | | | | KRAB box domain: S5- | | |
| | | | | | Y75 | | |
| 56 | 2228991 | 231 | T167 S213 T99 | N97 | Signal peptide: M1-I29 | | MOTIFS |
| | | | S186 T223 S10 | | Prenyl group binding | | SPSCAN |
| | | | S35 S67 T99 | | site (CAAX box) | | BLIMPS_PFAM |
| | | | | | T228-D231 | | |
| | | | | | Zinc finger domain: | | |
| | | | | | C166-H176 | | |
| 57 | 2241206 | 456 | S37 S404 S406 | N430 | RNA-binding RGG-box | g 1177636 | MOTIFS |
| | | | T183 T205 T212 | | domain I392-G452 | transcriptional | BLAST_GENBANK |
| | | | T264 T295 T300 | | | activator SPO8 | BLAST_DOMO |
| | | | T352 T50 T72 | | | | |
| 58 | 2259590 | 159 | S87 T96 S11 S24 | | Zinc finger protein | g506502 NK10 Zinc | MOTIFS |
| | | | S25 T118 T146 | | motif: F88-G126 | finger repressor | BLAST_GENBANK |
| | | | | | KRAB box domain: V86- | protein [Mus musculus] | BLIMPS_PRODOM |
| | | | | | C156 | 2.9e−15 47% ID aa 75-159 | BLAST_PRODOM |
| | | | | | | Lange, R. et al. DNA | BLAST_DOMO |
| | | | | | | Cell Biol 1995 | |
| | | | | | | Nov; 14(11): 971-81 | |
| 59 | 2307537 | 260 | T66 S124 S182 | N36 N195 | | g4325209 endocrine | MOTIFS |
| | | | S197 T7 S56 S77 | | | regulator | BLAST_GENBANK |
| 60 | 2440675 | 352 | S118 S153 S222 | | A193 ATP/GTP-binding | g 4165083 growth | MOTIFS |
| | | | S255 S317 S9 | | site motif A (P-loop) | factor independence-1B | BLAST_GENBANK |
| | | | T250 T289 T321 | | A193-T201 | Zinc finger protein | BLIMPS_PRINTS |
| | | | | | Zinc finger C2H2 type | Rodel, B. et al. | BLIMPS_PRODOM |
| | | | | | domain C165-H186 | Genomics 1998 Dec | BLAST_PRODOM |
| | | | | | C194-H215, C244-H265, | 15; 54 (3): 580-2 | BLAST_DOMO |
| | | | | | C272-H293, C300-H321, | | |
| | | | | | C328-H349 | | |
| 61 | 2463542 | 467 | S126 S132 S200 | N114 | Zinc finger C2H2 type | | MOTIFS |
| | | | S214 S220 S249 | N335 | domain: C6-H28 | | BLAST_GENBANK |
| | | | S393 S404 S419 | N354 | | | HMMER_PFAM |
| | | | S42 S430 S435 | | | | BLIMPS_BLOCKS |
| | | | S449 S77 T105 | | | | BLIMPS_PRINTS |
| | | | T14 T253 T397 | | | | |
| | | | T454 T81 Y313 | | | | |
| 62 | 2486031 | 550 | S115 S272 S317 | N238 | Homeobox domain: L70- | g 1504088 DNA-binding | MOTIFS |
| | | | S429 S441 S444 | N249 | I112 | protein | BLAST_GENBANK |
| | | | S62 S81 T302 | | | | BLIMPS_BLOCKS |
| | | | T360 T364 | | | | |
| 63 | 2493052 | 450 | S272 S293 S368 | N122 | Signal peptide: M1-S32 | g9230649 | MOTIFS |
| | | | S41 S411 S413 | N167 | Cytochrome c family | zinc finger protein | BLAST_GENBANK |
| | | | T108 T232 T238 | N185 | heme-binding site: | 277 [Homo sapiens] | HMMER_PFAM |
| | | | T374 T409 T418 | N403 | C359-V364 | Liang, H. et al. | BLIMPS_BLOCKS |
| | | | T433 T50 Y262 | | Zinc finger C2H2 type | (2000) Genomics | |
| | | | Y396 Y99 | | domain: | 66: 226-228 | |
| | | | | | C226-H248, C357-H381 | | |
| 64 | 2512074 | 378 | S132 S3 T9 T18 | N120 | Zinc finger C2H2 type | g 881564 ZNF157 | MOTIFS |
| | | | S77 S328 T182 | N150 | domain: C161-H181, | | BLAST_GENBANK |
| | | | S197 T279 S365 | N180 | C189-H209, C217-H237, | | HMMER_PFAM |
| | | | | N255 | C245-H265, C273-H293, | | BLIMPS_BLOCKS |
| | | | | N310 | C301-H321, C329-H349, | | BLIMPS_PRINTS |
| | | | | | C357-H377 | | BLIMPS_PRODOM |
| | | | | | Zinc finger protein | | BLAST_PRODOM |
| | | | | | domain: F10-G48 | | BLAST_DOMO |
| | | | | | KRAB box domain: Q5- | | |
| | | | | | P79 | | |
| 65 | 2646274 | 233 | S14 T34 T127 | N53 N67 | Protein I | g10046714 | MOTIFS |
| | | | | | transcription | transcription | BLAST_GENBANK |
| | | | | | initiation factor F84- | initiation factor IA | BLAST_PRODOM |
| | | | | | Y230 | protein [Homo sapiens] | |
| 66 | 2672566 | 102 | T66 | N11 T99 | | g 3220232 polyhomeotic | MOTIFS |
| | | | | | | Z protein | |
| | | | | | | Hemenway, C. S. et al. | |
| | | | | | | (1998) Oncogene | |
| | | | | | | 16: 2541-2547 | |
| | | | | | | Haluska, P. et al. | |
| | | | | | | (1999) Nucleic Acids | |
| | | | | | | Res. 27: 2538-2544 | |
| 67 | 2689674 | 287 | T25 T232 S32 | | Eukaryotic putative | g 1899188 DNA binding | MOTIFS |
| | | | S122 | | RNA-binding region | protein ACBF | BLAST_GENBANK |
| | | | | | RNP-1 signature: K137- | AC-rich binding factor | HMMER_PFAM |
| | | | | | D146, L98-F116 | | BLIMPS_BLOCKS |
| | | | | | RNA recognition motif: | | BLAST_PRODOM |
| | | | | | L98-L170, L5-K77 | | BLAST_DOMO |
| 68 | 2703282 | 208 | S11 S23 S117 | | | g5712754 | MOTIFS |
| | | | Y124 | | | sex comb on midleg- | BLAST_GENBANK |
| | | | | | | like-1 protein [Homo | |
| | | | | | | sapiens] | |
| | | | | | | van de Vosse, E. et | |
| | | | | | | al. (1998) Genomics | |
| | | | | | | 49: 96-102 | |
| 69 | 2738293 | 177 | S71 T43 S5 T115 | | | g11907923 | MOTIFS |
| | | | | | | enhancer of polycomb | BLAST_GENBANK |
| | | | | | | [Homo sapiens] | |
| | | | | | | Shimono, Y. et al. | |
| | | | | | | (2000) | |
| | | | | | | J. Biol. Chem. | |
| | | | | | | 275: 39411-39419 | |
| 70 | 2772776 | 179 | T173 S29 S39 | | Zinc finger protein | g6942207 | MOTIFS |
| | | | T63 T106 | | motif: P104-A166 | PPARgamma cofactor 2 | BLAST_GENBANK |
| | | | | | | [Mus musculus] | BLAST_PRODOM |
| | | | | | | Castillo, G. C. et al. | BLAST_DOMO |
| | | | | | | (1999) EMBO J. | |
| | | | | | | 18: 3676-3687 | |
| 71 | 2774476 | 212 | S132 S159 S196 | | RBP-J Kappa | g 2052119 | MOTIFS |
| | | | S20 S201 S31 | | Recombination signal | transcription factor | BLAST_GENBANK |
| | | | S45 T136 T205 | | motif: P39-D206 | RBP-L | BLAST_PRODOM |
| | | | T68 | | | | |
| 72 | 2804624 | 256 | S103 S202 S238 | N6 N101 | MAF-1 nuclear matrix | g3786409 contains | MOTIFS |
| | | | S244 S33 S7 S85 | N132 | protein motif: G82- | similarity to | BLAST_GENBANK |
| | | | S89 T112 T212 | | T210 | Saccharomyces | BLAST_PRODOM |
| | | | T245 T99 | | | cerevisiae MAF-1 | |
| | | | | | | protein | |
| 73 | 2848225 | 475 | S179 S180 S239 | N12 N93 | Zinc finger C2H2 type | g 930123 zinc finger | MOTIFS |
| | | | S24 S295 S378 | | domain: F6-R44, C171- | protein | BLAST_GENBANK |
| | | | S434 T14 T142 | | Q191, C199-H219, C227- | | HMMER_PFAM |
| | | | T164 T282 T332 | | H247, C255-H275, C283- | | BLIMPS_BLOCKS |
| | | | T36 Y136 Y268 | | H303, C311-H331, C339- | | BLIMPS_PRINTS |
| | | | | | H358, C366-H386, C394- | | BLIMPS_PRODOM |
| | | | | | H415, C422-H442, C450- | | BLAST_PRODOM |
| | | | | | H470 | | BLAST_DOMO |
| | | | | | Zinc finger 136: W37- | | |
| | | | | | Q145 | | |
| | | | | | Zinc finger 137: S259- | | |
| | | | | | R335 | | |
| | | | | | KRAB box: M1-D76 | | |
| 74 | 2882241 | 206 | S164 S166 S57 | | Helix-loop-helix DNA- | g 1184157 Max- | MOTIFS |
| | | | S161 Y29 | | binding domain: G58- | interacting | BLAST_GENBANK |
| | | | | | E110, H27-D165 | transcriptional | BLAST_PFAM |
| | | | | | Myc-type helix-loop- | repressor | BLAST_DOMO |
| | | | | | helix motif: E66-K81, | | |
| | | | | | C90-E110 | | |
| 75 | 2939011 | 596 | S152 S203 S212 | N84 N510 | | g 5081374 | MOTIFS |
| | | | S244 S272 S477 | | | glucocorticoid | BLAST_GENBANK |
| | | | S481 S516 S536 | | | modulatory element | |
| | | | T121 T164 T200 | | | binding protein-1 | |
| | | | T204 T229 T339 | | | | |
| | | | T361 T363 T396 | | | | |
| | | | T537 T543 | | | | |
| 76 | 2947188 | 644 | S116 S207 S22 | N66 | ATP/GTP-binding site | g5441615 zinc finger | MOTIFS |
| | | | S403 S488 S85 | N190 | motif A (P-loop): | protein | BLAST_GENBANK |
| | | | T110 T487 T52 | N265 | G142-S149 | | HMMER_PFAM |
| | | | T612 | N376 | Zinc finger C2H2 type | | BLIMPS_BLOCKS |
| | | | | | domain: C199-H219, | | BLIMPS_PRINTS |
| | | | | | C227-H247, C255-H275, | | BLIMPS_PRODOM |
| | | | | | C283-H303, C311-H331, | | BLAST_PRODOM |
| | | | | | C339-H359, C367-H387, | | |
| | | | | | C395-H415, C423-H443, | | |
| | | | | | C451-H471, C479-H499, | | |
| | | | | | C507-H527, C535-H555, | | |
| | | | | | C563-H583, C591-H611, | | |
| | | | | | C619-H639 | | |
| 77 | 3094001 | 194 | T59 T110 S27 | N17 | SSU72 start-site | g4156162 | MOTIFS |
| | | | S32 S183 Y65 | | selection protein: M1- | similar to yeast SSU72 | BLAST_GENBANK |
| | | | | | F194 | | BLAST_PRODOM |
| 78 | 3110061 | 536 | S21 S134 T157 | N132 | Zinc finger C2H2 type | g1017722 repressor | MOTIFS |
| | | | S214 T76 S83 | N380 | domain: C202-H222, | transcriptional factor | BLAST_GENBANK |
| | | | S252 S404 S462 | N389 | C230-H250, C258-H277, | | HMMER_PFAM |
| | | | | N445 | C286-H306, C314-H334, | | BLIMPS_BLOCKS |
| | | | | | C342-H362, C370-H390, | | BLIMPS_PRINTS |
| | | | | | C398-H418, C426-H446, | | BLIMPS_PRODOM |
| | | | | | C454-H474, C482-H502 | | BLAST_PRODOM |
| | | | | | Transcription factor | | BLAST_DOMO |
| | | | | | GATA zinc finger | | |
| | | | | | signature: T223-S240 | | |
| | | | | | Zinc finger signature: | | |
| | | | | | F13-G51, H330-C342 | | |
| 79 | 3146614 | 412 | S184 T158 T247 | | Signal peptide: M1-R29 | g2370560 putative | MOTIFS |
| | | | S402 | | Transcription | translational | BLAST_GENBANK |
| | | | | | regulation protein | repressor | SPSCAN |
| | | | | | domain: F3-L220 | | BLAST_PRODOM |
| | | | | | Leucine zipper motif: | | |
| | | | | | L80-L101 | | |
| 80 | 3295381 | 482 | S161 S216 S318 | | Zinc finger C2H2 type | g6118383 | MOTIFS |
| | | | S352 S385 S408 | | domain: | zinc finger protein | BLAST_GENBANK |
| | | | S456 S72 S99 | | C178-H198, C206-H226, | ZNF223 [Homo sapiens] | BLAST_PFAM |
| | | | T177 T18 T84 | | C234-H254, | | BLIMPS_BLOCKS |
| | | | T88 T9 T94 | | C262-H282, C290-H310, | | BLIMPS_PRINTS |
| | | | | | C346-H366, | | BLIMPS_PRODOM |
| | | | | | C374-H394, C402-H422 | | BLAST_PRODOM |
| | | | | | Zinc finger signature: | | BLAST_DOMO |
| | | | | | F10-G48 | | |
| | | | | | KRAB box: V8-G69 | | |
| 81 | 3364774 | 554 | S134 S183 S269 | N467 | ATP/GTP-binding site | g 3818515 zinc finger | MOTIFS |
| | | | S292 S307 S458 | | motif A (P-loop): | protein ZNF210 | BLAST_GENBANK |
| | | | S514 S62 S94 | | A505-S512 | | BLAST_PFAM |
| | | | T125 T16 T325 | | Zinc finger C2H2 type | | BLIMPS_BLOCKS |
| | | | T402 T497 | | domain: C310-H330, | | BLIMPS_PRINTS |
| | | | | | C338-H358, C366-H386, | | BLIMPS_PRODOM |
| | | | | | C394-H414, C422-H442, | | BLAST_PRODOM |
| | | | | | C450-H470, C478-H498, | | BLAST_DOMO |
| | | | | | C506-H526 | | |
| | | | | | KRAB box: V124-S183 | | |
| | | | | | Zinc finger signature: | | |
| | | | | | F126-P164 | | |
| 82 | 3397777 | 488 | S171 S235 S244 | N448 | Zinc finger C3HC4 | g11022688 | MOTIFS |
| | | | S271 S346 S356 | | type signature: | interferon-responsive | BLAST_GENBANK |
| | | | S417 S42 T153 | | C30-I40, C15-C59, V9- | finger protein 1 | HMMER_PFAM |
| | | | T178 T185 | | S64 | middle form [Homo | BLIMPS_BLOCKS |
| | | | | | Interleukin 2 | sapiens] | PROFILESCAN |
| | | | | | transcription down- | Orimo, A. et al. | BLIMPS_PFAM |
| | | | | | regulatory domain: | (2000) Genomics | BLAST_PRODOM |
| | | | | | T130-W333 | 69: 143-149 | BLAST_DOMO |
| | | | | | RFP Transforming | | |
| | | | | | protein: H67-G347 | | |
| 83 | 3403046 | 127 | T57 S31 S52 | | Signal peptide: M1-P19 | g 1890635 Jun | MOTIFS |
| | | | | | bZIP transcription | dimerization protein 1 | BLAST_GENBANK |
| | | | | | factors basic domain | JDP-1 | HMMER_PFAM |
| | | | | | signature: R40-K55, | | BLIMPS_BLOCKS |
| | | | | | E33-E97 | | BLAST_PRODOM |
| | | | | | FOS transforming | | BLAST_DOMO |
| | | | | | protein: Q28-K44, N45- | | |
| | | | | | D61, L63-L84 | | |
| | | | | | DNA-binding | | |
| | | | | | transcription factor: | | |
| | | | | | A17-L104 | | |
| | | | | | Leucine zipper motifs: | | |
| | | | | | L63-L84, L70-L91 | | |
| 84 | 3538506 | 532 | S143 S170 S185 | N280 | Signal peptide: M1- | g4056411 Human homolog | MOTIFS |
| | | | S191 S282 S283 | | A51 | of Mus musculus wizS | BLAST_GENBANK |
| | | | S327 S340 S457 | | C2H2 Zn finger domain: | protein | SPSCAN |
| | | | S49 S497 S72 | | C3-H23, C109-H129, | | HMMER-PFAM |
| | | | S99 T147 T156 | | C293-H313, C463-H483, | | BLIMPS-BLOCKS |
| | | | T223 T30 T331 | | C3-H19 | | MOTIFS |
| | | | T449 T501 T71 | | | | |
| | | | T86 | | | | |
| 85 | 3575519 | 353 | S110 S194 S210 | | C3HC4 Zn finger | g9945010 | MOTIFS |
| | | | S240 S256 S266 | | domain: C23-C78, C39- | RING-finger protein | BLAST_GENBANK |
| | | | S285 T189 T198 | | A48, K19-G88 | MURF [Mus musculus] | HMMER-PFAM |
| | | | T278 T326 T60 | | RFP (Zn finger | Spencer, J. A. et al. | PROFILESCAN |
| | | | T77 | | oncogenic protein): | (2000) J. Cell Biol. | BLAST-DOMO |
| | | | | | K106-K291 | 150: 771-784 | MOTIFS |
| 86 | 3598694 | 407 | S16 S76 S151 | | | g 5668703 XDRP1 | MOTIFS |
| | | | S315 T390 | | | | BLAST_GENBANK |
| | | | | | | | BLIMPS-BLOCKS |
| 87 | 3638819 | 350 | S199 S212 S236 | N12 N210 | Transmembrane domain: | g1020145 DNA binding | MOTIFS |
| | | | Y156 Y184 | | L310-F330 | protein | BLAST_GENBANK |
| | | | | | C2H2 Zn finger domain: | | HMMER |
| | | | | | G82-A264, Y114-H136, | | HMMER-PFAM |
| | | | | | C88-H108, Y142-H164, | | BLIMPS-PRINTS |
| | | | | | F170-H192, Y198-H220, | | BLAST-PRODOM |
| | | | | | F226-H248, P113-S126, | | BLAST-DOMO |
| | | | | | L129-G138 | | MOTIFS |
| 88 | 3717139 | 215 | S108 S198 S70 | N42 N196 | Homeobox domain: K33- | g 2632119 Splice | MOTIFS |
| | | | T193 | | N96, Q36-E95, E50- | variant of homeobox | BLAST_GENBANK |
| | | | | | A112, R79-N96, T58- | gene Prx3A | HMMER-PFAM |
| | | | | | L69, L73-R92 | alternative N-terminal | PROFILESCAN |
| | | | | | | region | BLIMPS-BLOCKS |
| | | | | | | | BLIMPS-PRINTS |
| | | | | | | | BLAST- PRODOM |
| | | | | | | | BLAST-DOMO |
| 89 | 3892962 | 489 | S227 S43 S113 | N110 | C2H2 Zn finger domain: | g 488555 zinc finger | MOTIFS |
| | | | T230 T97 S196 | N200 | C132-H152, L145-G154, | protein ZNF135 | BLAST_GENBANK |
| | | | T392 | N308 | C160-H180, C188-H208, | | HMMER-PFAM |
| | | | | N319 | C216-H236, C244-H264, | | BLIMPS-BLOCKS |
| | | | | N366 | C272-H292, C300-H320, | | BLIMPS-PRINTS |
| | | | | N450 | P325-S338, C328-H348, | | BLAST-PRODOM |
| | | | | N480 | C356-H376, C384-H404, | | BLAST-DOMO |
| | | | | | C412-H432, C440-H460, | | |
| | | | | | C468-H488, G108-H488 | | |
| | | | | | Zn finger protein | | |
| | | | | | domain: K127-H488 | | |
| 90 | 4153521 | 399 | S112 S14 S146 | N139 | C2H2 Zn finger domain: | g7688669 | MOTIFS |
| | | | S157 S164 S176 | N155 | C315-H335, C231-H251, | zinc finger protein | BLAST_GENBANK |
| | | | S364 S70 S75 | N177 | C343-H363, C287-H307, | ZNF140-like protein | HMMER-PFAM |
| | | | S83 T119 T123 | N184 | C203-H223, C259-H279, | [Homo sapiens] | BLIMPS-BLOCKS |
| | | | T133 T202 T5 | | P340-S353, L216-G225, | | BLIMPS-PRINTS |
| | | | T84 | | C371-H391 | | BLIMPS_PRODOM |
| | | | | | Zn finger protein | | BLAST-PRODOM |
| | | | | | domain: V4-W77, F6- | | BLAST-DOMO |
| | | | | | G44 | | |
| | | | | | KRAB box domain: S2- | | |
| | | | | | W73, V4-D72 | | |
| 91 | 4585038 | 309 | S106 S270 S293 | | transcriptional | g 4960159 GC-rich | MOTIFS |
| | | | S51 S52 S75 S94 | | repressor DNA binding | sequence DNA-binding | BLAST_GENBANK |
| | | | T132 T172 T198 | | signature: D45-K275 | factor candidate | BLAST-PRODOM |
| | | | T205 T237 T301 | | | | |
| | | | T31 T40 T57 | | | | |
| 92 | 4674640 | 361 | S28 S30 S352 | N288 | Type I antifreeze | g 3779240 zinc finger | MOTIFS |
| | | | S56 T170 T176 | | protein signature: | protein | BLAST_GENBANK |
| | | | T206 T77 Y233 | | Q253-F270 | | BLIMPS-PRINTS |
| | | | Y68 | | | | |
| 93 | 4676066 | 540 | S115 S135 S151 | N222 | Signal peptide: M1- | g 3916727 estrogen- | MOTIFS |
| | | | S202 S301 S34 | | S29 | responsive B box | BLAST_GENBANK |
| | | | S39 S405 S490 | | RFP (Zn finger | protein | SPSCAN |
| | | | S497 T368 T508 | | oncogenic protein): | | BLIMPS_PRINTS |
| | | | | | R381-I526 | | BLAST-DOMO |
| | | | | | Adrenomedullin | | |
| | | | | | signature: R111-A128 | | |
| 94 | 4830687 | 84 | | | Signal peptide: M1- | g7649253 | MOTIFS |
| | | | | | A66 | hepatocellular | BLAST_GENBANK |
| | | | | | C3HC4 Zn finger | carcinoma associated | HMMER_PFAM |
| | | | | | domain: C33-E79, C51- | ring finger protein | SPSCAN |
| | | | | | C76, N19-K81 | [Homo sapiens] | PROFILESCAN |
| | | | | | Glycoprotein hormone | | BLAST-DOMO |
| | | | | | signature: M1-H58 | | |
| 95 | 4880891 | 1312 | S105 S1060 | N294 | ARID (AT-Rich | g 5257005 Rb binding | MOTIFS |
| | | | S1063 S1067 | N432 | Interaction Domain) | protein homolog | BLAST_GENBANK |
| | | | S1128 S1129 | N755 | DNA binding domain: | | HMMER-PFAM |
| | | | S1135 S1153 | N856 | E303-V413 | | BLAST-PRODOM |
| | | | S1159 S1181 | N859 | Retinoblastoma binding | | BLAST-DOMO |
| | | | S1208 S1222 | N910 | protein: T742-R1312 | | |
| | | | S1249 S157 S158 | N1151 | | | |
| | | | S159 S17 S216 | N1226 | | | |
| | | | S274 S276 S295 | | | | |
| | | | S296 S47 S471 | | | | |
| | | | S483 S527 S591 | | | | |
| | | | S595 S656 S666 | | | | |
| | | | S680 S712 S713 | | | | |
| | | | S717 S736 S750 | | | | |
| | | | S758 S815 S860 | | | | |
| | | | S861 S862 S888 | | | | |
| | | | S945 S947 T100 | | | | |
| | | | T1025 T1034 | | | | |
| | | | T1046 T1228 | | | | |
| | | | T126 T1293 T140 | | | | |
| | | | T31 T41 T481 | | | | |
| | | | T507 T508 T531 | | | | |
| | | | T793 T801 T811 | | | | |
| | | | T812 T876 T939 | | | | |
| | | | T971 Y655 Y75 | | | | |
| | | | Y89 Y9 | | | | |
| 96 | 4909754 | 504 | S109 S181 S304 | N309 | Transcription factor- | g476099 transcription | MOTIFS |
| | | | S357 S36 S384 | N355 | like domain: T20-L120 | factor LSF | BLAST_GENBANK |
| | | | S389 S417 T194 | N421 | Lymphoid transcription | | BLIMPS_PRINTS |
| | | | T212 T246 T255 | | factor ENL: P10-N209 | | BLAST-PRODOM |
| | | | T323 T333 T365 | | P245 purinoceptor | | BLAST-DOMO |
| | | | T490 Y221 Y262 | | signature: F121-K131 | | |
| | | | Y85 | | | | |
| 97 | 4911931 | 227 | T190 S191 T157 | | Transcription factor- | g3878581 Similar to | MOTIFS |
| | | | Y62 | | like domain: T20-L120 | Human AF-9 leukemia | BLAST_GENBANK |
| | | | | | Lymphoid transcription | protein | BLAST-PRODOM |
| | | | | | factor ENL: P10-N209 | | BLAST-DOMO |
| | | | | | P245 purinoceptor | | |
| | | | | | signature: F121-K131 | | |
| 98 | 4920433 | 233 | S43 S50 T62 S77 | N122 | Signal peptide: M1- | | SPSCAN |
| | | | S110 S131 T165 | N192 | A33 | | MOTIFS |
| | | | S17 T69 S194 | | LysR helix-turn helix | | |
| | | | Y191 | | domain: T97-N122 | | |
| 99 | 5042113 | 511 | S176 S203 S276 | | Signal peptide: M1- | | MOTIFS |
| | | | S278 S430 S436 | | A34 | | SPSCAN |
| | | | S455 S56 S99 | | Brain natriuretic | | BLIMPS-PRINTS |
| | | | T12 T173 T239 | | peptide: A481-Q499 | | |
| | | | T247 T274 T372 | | | | |
| | | | T449 T504 T509 | | | | |
| | | | Y79 | | | | |
| 100 | 5083853 | 247 | S102 S110 S123 | | C-type natriuretic | g 4519621 OASIS | MOTIFS |
| | | | S19 S190 S58 | | peptide: S44-D54 | (transcription factor) | BLAST_GENBANK |
| | | | S84 T150 T163 | | | protein | |
| | | | T212 | | | | |
| 101 | 5283981 | 276 | S160 T68 T126 | N20 | C2H2 Zn finger domain: | g 5001720 odd-skipped | MOTIFS |
| | | | T168 T193 | | K170-H190, F172-H194, | related 1 protein | BLAST_GENBANK |
| | | | | | C174-H194, L187-D196, | | HMMER-PFAM |
| | | | | | E191-H246, Y200-H222, | | BLIMPS-BLOCKS |
| | | | | | F228-H250, C202-H218, | | BLIMPS-PRINTS |
| | | | | | S223-Q252, P227-S240. | | BLAST-PRODOM |
| | | | | | C230-H250 | | BLAST-DOMO |
| 102 | 5510549 | 220 | S66 T144 S173 | N202 | C3HC4 Zn finger | g9759106 | MOTIFS |
| | | | T67 S153 | | domain: C168-C208, | contains similarity to | BLAST_GENBANK |
| | | | | | E164-A219, C168-D211 | C3HC4-type RING zinc | HMMER-PFAM |
| | | | | | | finger protein | PROFILESCAN |
| | | | | | | Sato, S. et al. (1997) | BLAST-DOMO |
| | | | | | | DNA Res. 4: 215-230 | |
| 103 | 5544862 | 608 | S16 S25 S326 | N29 | Small proline rich | | MOTIFS |
| | | | S401 S416 S423 | N251 | protein DNA binding | | BLIMPS-PRINTS |
| | | | S424 S44 S481 | N538 | signature: | | |
| | | | S51 S517 S518 | | E48-P57, P230-P238 | | |
| | | | S530 S543 S553 | | Leucine zipper: L63- | | |
| | | | S566 S574 S597 | | L84 | | |
| | | | T118 T182 T256 | | | | |
| | | | T402 T437 T494 | | | | |
| | | | Y552 Y579 | | | | |
| 104 | 5573394 | 653 | G650 S299 S371 | N115 | Nonstructural | | MOTIFS |
| | | | S499 S552 S593 | N220 | polyprotein domain: | | BLAST_DOMO |
| | | | S599 S78 T240 | N293 | L118-K284 | | |
| | | | T262 T270 T300 | N597 | | | |
| | | | T381 T432 T525 | | | | |
| | | | T53 T57 T9 | | | | |
| 105 | 5850840 | 154 | T9 S19 S25 T30 | | C3HC4 Zn finger | g 3873857 similar to | MOTIFS |
| | | | T63 S138 S149 | | domain: C99-C139, K95- | C3HC4 type zinc finger | BLAST_GENBANK |
| | | | S21 S92 | | S150 | | HMMER-PFAM |
| | | | | | | | PROFILESCAN |
| 106 | 5942936 | 337 | T8 S10 S12 S67 | N25 N65 | Helix-loop-helix DNA | g 5059323 hairy and | MOTIFS |
| | | | T77 S138 T214 | | binding domain: R49- | enhancer of split | BLAST_GENBANK |
| | | | S84 T162 | | E132, K51-Q104, E57- | related-1 | HMMER-PFAM |
| | | | | | R72, S84-Q104, E88- | | BLIMPS-BLOCKS |
| | | | | | L103 | | BLAST-DOMO |
| 107 | 5951431 | 535 | S152 S201 S212 | N6 N87 | Signal peptide: M1- | g9651765 | MOTIFS |
| | | | S254 S256 S287 | N137 | R54 | zinc finger protein | BLAST_GENBANK |
| | | | S348 S351 S354 | | GATA-type Zn finger | 289 [Mus musculus] | HMMER_PFAM |
| | | | S378 S385 S409 | | domain: A19-W74, M1- | | SPSCAN |
| | | | S414 S428 S475 | | H95 | | BLAST-PRODOM |
| | | | S527 T22 T418 | | | | BLAST-DOMO |
| | | | T66 T98 Y210 | | | | |
| | | | Y459 | | | | |

TABLE 3

| Nucleotide SEQ | | Tissue Expression | Disease or Condition | |
|---|---|---|---|---|
| ID NO: | | (Fraction of Total) | (Fraction of Total) | Vector |
| 108 | 095210 | Reproductive (0.257) | Cancer (0.429) | PBLUESCRIPT |
| | | Hematopoietic/Immune (0.200) | Inflammation (0.257) | |
| | | Nervous (0.171) | Cell Proliferation (0.143) | |
| 109 | 157953 | Reproductive (0.293) | Cancer (0.483) | PBLUESCRIPT |
| | | Hematopoietic/Immune (0.207) | Cell Proliferation (0.310) | |
| | | Gastrointestinal (0.172) | Inflammation (0.224) | |
| 110 | 159196 | Reproductive (0.296) | Cancer (0.444) | PBLUESCRIPT |
| | | Cardiovascular (0.222) | Inflammation (0.370) | |
| | | Hematopoietic/Immune (0.111) | Cell Proliferation (0.222) | |
| | | Gastrointestinal (0.111) | | |
| | | Urologic (0.111) | | |
| 111 | 343338 | Hematopoietic/Immune (0.300) | Cancer (0.380) | PBLUESCRIPT |
| | | Nervous (0.260) | Inflammation (0.320) | |
| | | Reproductive (0.140) | Cell Proliferation (0.220) | |
| 112 | 402386 | Hematopoietic/Immune (0.381) | Inflammation (0.476) | PBLUESCRIPT |
| | | Reproductive (0.190) | Cancer (0.333) | |
| | | Gastrointestinal (0.143) | | |
| | | Nervous (0.143) | | |
| 113 | 456487 | Reproductive (0.248) | Cancer (0.488) | PBLUESCRIPT |
| | | Nervous (0.198) | Inflammation (0.207) | |
| | | Gastrointestinal (0.132) | Cell Proliferation (0.165) | |
| 114 | 490256 | Developmental (0.231) | Cancer (0.231) | PBLUESCRIPT |
| | | Reproductive (0.231) | Cell Proliferation (0.231) | |
| | | Endocrine (0.154) | Inflammation (0.231) | |
| | | Hematopoietic/Immune (0.154) | | |
| | | Gastrointestinal (0.154) | | |
| 115 | 494740 | Gastrointestinal (0.209) | Inflammation (0.395) | PBLUESCRIPT |
| | | Nervous (0.209) | Cancer (0.302) | |
| | | Hematopoietic/Immune (0.186) | Cell Proliferation (0.209) | |
| 116 | 507475 | Reproductive (0.246) | Cancer (0.426) | PBLUESCRIPT |
| | | Hematopoietic/Immune (0.180) | Cell Proliferation (0.230) | |
| | | Gastrointestinal (0.148) | Inflammation (0.230) | |
| 117 | 531581 | Hematopoietic/Immune (0.231) | Cancer (0.385) | PSPORT1 |
| | | Reproductive (0.231) | Cell Proliferation (0.231) | |
| | | Nervous (0.154) | Inflammation (0.205) | |
| 118 | 675190 | Reproductive (0.389) | Cancer (0.722) | PSPORT1 |
| | | Nervous (0.278) | Inflammation (0.111) | |
| | | Cardiovascular (0.111) | Trauma (0.111) | |
| | | Urologic (0.111) | | |
| 119 | 685434 | Reproductive (0.333) | Cancer (0.556) | PSPORT1 |
| | | Nervous (0.194) | Inflammation (0.278) | |
| | | Cardiovascular (0.111) | Cell Proliferation (0.111) | |
| | | Hematopoietic/Immune (0.111) | | |
| 120 | 788663 | Reproductive (0.303) | Cancer (0.455) | PSPORT1 |
| | | Cardiovascular (0.182) | Inflammation (0.303) | |
| | | Hematopoietic/Immune (0.152) | Cell Proliferation (0.212) | |
| 121 | 870100 | Reproductive (0.298) | Cancer (0.660) | PSPORT1 |
| | | Nervous (0.170) | Inflammation (0.170) | |
| | | Cardiovascular (0.128) | Cell Proliferation (0.149) | |
| 122 | 879500 | Reproductive (0.203) | Cancer (0.373) | PSPORT1 |
| | | Gastrointestinal (0.153) | Inflammation (0.322) | |
| | | Hematopoietic/Immune (0.136) | Cell Proliferation (0.203) | |
| 123 | 975377 | Reproductive (0.215) | Cancer (0.418) | PSPORT1 |
| | | Nervous (0.177) | Inflammation (0.291) | |
| | | Hematopoietic/Immune (0.152) | Cell Proliferation (0.127) | |
| 124 | 1208721 | Reproductive (0.282) | Cancer (0.471) | PSPORT1 |
| | | Nervous (0.200) | Inflammation (0.282) | |
| | | Hematopoietic/Immune (0.141) | Cell Proliferation (0.141) | |
| 125 | 1234329 | Reproductive (0.277) | Cancer (0.553) | pINCY |
| | | Nervous (0.191) | Cell Proliferation (0.234) | |
| | | Cardiovascular (0.128) | Inflammation (0.213) | |
| | | Hematopoietic/Immune (0.128) | | |
| 126 | 1238747 | Hematopoietic/Immune (0.283) | Cancer (0.400) | PSPORT1 |
| | | Gastrointestinal (0.167) | Inflammation (0.300) | |
| | | Reproductive (0.150) | Trauma (0.117) | |
| | | | Cell Proliferation (0.117) | |
| 127 | 1265980 | Nervous (0.900) | Cell Proliferation (0.400) | pINCY |
| | | Developmental (0.100) | Inflammation (0.200) | |
| | | | Neurological (0.200) | |
| 128 | 1297333 | Developmental (0.273) | Cell Proliferation (0.273) | pINCY |
| | | Reproductive (0.273) | Inflammation (0.273) | |
| | | Hematopoietic/Immune (0.273) | Cancer (0.182) | |
| 129 | 1312824 | Reproductive (0.238) | Cancer (0.429) | pINCY |
| | | Hematopoietic/Immune (0.222) | Inflammation (0.238) | |
| | | Gastrointestinal (0.159) | Cell Proliferation (0.175) | |
| 130 | 1359294 | Reproductive (0.219) | Cancer (0.438) | pINCY |
| | | Nervous (0.157) | Inflammation (0.247) | |
| | | Gastrointestinal (0.145) | Cell Proliferation (0.188) | |
| 131 | 1377380 | Reproductive (0.385) | Cancer (0.538) | pINCY |
| | | Developmental (0.231) | Cell Proliferation (0.385) | |
| | | Hematopoietic/Immune (0.231) | Inflammation (0.154) | |
| 132 | 1383473 | Reproductive (0.318) | Cancer (0.515) | pINCY |
| | | Nervous (0.182) | Inflammation (0.288) | |
| | | Hematopoietic/Immune (0.121) | Cell Proliferation (0.197) | |
| 133 | 1388860 | Cardiovascular (0.167) | Cancer (0.444) | pINCY |
| | | Nervous (0.167) | Inflammation (0.222) | |
| | | Reproductive (0.167) | Cell Proliferation (0.167) | |
| | | | Trauma (0.167) | |
| 134 | 1395322 | Nervous (0.261) | Cancer (0.478) | pINCY |
| | | Reproductive (0.261) | Inflammation (0.304) | |
| | | | Cell Proliferation (0.130) | |
| | | | Trauma (0.130) | |
| 135 | 1419370 | Reproductive (0.290) | Cancer (0.522) | pINCY |
| | | Nervous (0.246) | Cell Proliferation (0.188) | |
| | | Gastrointestinal (0.116) | Inflammation (0.130) | |
| 136 | 1429773 | Reproductive (0.255) | Cancer (0.521) | pINCY |
| | | Gastrointestinal (0.160) | Inflammation (0.191) | |
| | | Cardiovascular (0.128) | Cell Proliferation (0.170) | |
| 137 | 1470820 | Reproductive (0.231) | Cancer (0.385) | pINCY |
| | | Developmental (0.154) | Cell Proliferation (0.231) | |
| | | Gastrointestinal (0.154) | Inflammation (0.231) | |
| | | Hematopoietic/Immune (0.154) | | |
| | | Nervous (0.154) | | |
| | | Gastrointestinal (0.154) | | |
| 138 | 1483455 | Nervous (0.222) | Cancer (0.422) | pINCY |
| | | Urologic (0.156) | Cell Proliferation (0.244) | |
| | | Cardiovascular (0.111) | Inflammation (0.244) | |
| | | Developmental (0.111) | | |
| | | Reproductive (0.111) | | |
| 139 | 1527064 | Reproductive (0.262) | Cancer (0.481) | PBLUESCRIPT |
| | | Nervous (0.169) | Cell Proliferation (0.257) | |
| | | Cardiovascular (0.131) | Inflammation (0.224) | |
| 140 | 1557491 | Nervous (0.222) | Cancer (0.444) | pINCY |
| | | Reproductive (0.222) | Neurological (0.167) | |
| | | Cardiovascular (0.167) | Cell Proliferation (0.111) | |
| | | | Inflammation (0.111) | |
| 141 | 1576862 | Gastrointestinal (0.280) | Cancer (0.480) | pINCY |
| | | Hematopoietic/Immune (0.240) | Inflammation (0.320) | |
| | | Nervous (0.160) | Cell Proliferation (0.120) | |
| 142 | 1609731 | Gastrointestinal (0.286) | Cancer (0.429) | pINCY |
| | | Nervous (0.286) | Cell Proliferation (0.286) | |
| | | Cardiovascular (0.143) | Neurological (0.143) | |
| | | Developmental (0.143) | Trauma (0.143) | |
| | | Urologic (0.143) | | |
| 143 | 1674538 | Nervous (0.364) | Cancer (0.364) | pINCY |
| | | Cardiovascular (0.364) | Cell Proliferation (0.182) | |
| | | Gastrointestinal (0.182) | | |
| 144 | 1675287 | Reproductive (0.373) | Cancer (0.492) | pINCY |
| | | Hematopoietic/Immune (0.169) | Inflammation (0.305) | |
| | | Urologic (0.119) | Cell Proliferation (0.153) | |
| 145 | 1693903 | Reproductive (0.212) | Cancer (0.434) | pINCY |
| | | Hematopoietic/Immune (0.186) | Inflammation (0.327) | |
| | | Nervous (0.177) | Cell Proliferation (0.257) | |
| 146 | 1702962 | Reproductive (0.389) | Cancer (0.556) | pINCY |
| | | Cardiovascular (0.167) | Trauma (0.222) | |
| | | Gastrointestinal (0.167) | | |
| 147 | 1712916 | Reproductive (1.000) | Cancer (1.000) | pINCY |
| 148 | 1748313 | Nervous (0.265) | Cancer (0.456) | pINCY |
| | | Reproductive (0.162) | Inflammation (0.279) | |
| | | Hematopoietic/Immune (0.147) | Cell Proliferation (0.176) | |
| 149 | 1754833 | Hematopoietic/Immune (0.208) | Inflammation (0.377) | pINCY |
| | | Gastrointestinal (0.189) | Cancer (0.358) | |
| | | Nervous (0.151) | Cell Proliferation (0.170) | |
| 150 | 1798701 | Nervous (0.237) | Cancer (0.449) | pINCY |
| | | Reproductive (0.212) | Inflammation (0.237) | |
| | | Gastrointestinal (0.119) | Cell Proliferation (0.178) | |
| 151 | 1842496 | Reproductive (0.254) | Cancer (0.500) | PSPORT1 |
| | | Nervous (0.187) | Cell Proliferation (0.224) | |
| | | Gastrointestinal (0.119) | Inflammation (0.149) | |
| 152 | 1868613 | Hematopoietic/Immune (0.286) | Cell Proliferation (0.486) | pINCY |
| | | Reproductive (0.257) | Cancer (0.400) | |
| | | Cardiovascular (0.114) | Inflammation (0.286) | |
| | | Gastrointestinal (0.114) | | |
| 153 | 1870609 | Nervous (0.207) | Cancer (0.439) | pINCY |
| | | Reproductive (0.195) | Inflammation (0.244) | |
| | | Gastrointestinal (0.159) | Cell Proliferation (0.171) | |
| 154 | 1871961 | Reproductive (0.268) | Cancer (0.474) | pINCY |
| | | Nervous (0.196) | Cell Proliferation (0.247) | |
| | | Hematopoietic/Immune (0.113) | Inflammation (0.165) | |
| 155 | 1876258 | Hematopoietic/Immune (0.600) | Inflammation (0.400) | pINCY |
| | | Cardiovascular (0.200) | Trauma (0.200) | |
| | | Reproductive (0.100) | Cancer (0.100) | |
| | | Gastrointestinal (0.100) | | |
| 156 | 1929822 | Reproductive (0.255) | Cancer (0.479) | pINCY |
| | | Nervous (0.160) | Inflammation (0.223) | |
| | | Hematopoietic/Immune (0.128) | Cell Proliferation (0.213) | |
| | | Gastrointestinal (0.128) | | |
| 157 | 1970095 | Nervous (0.205) | Cancer (0.385) | PBLUESCRIPT |
| | | Reproductive (0.205) | Inflammation (0.256) | |
| | | Cardiovascular (0.133) | Cell Proliferation (0.159) | |
| 158 | 1975473 | Gastrointestinal (0.464) | Cancer (0.536) | pINCY |
| | | Reproductive (0.250) | Inflammation (0.214) | |
| | | | Cell Proliferation (0.179) | |
| 159 | 1976527 | Reproductive (0.247) | Cancer (0.466) | pINCY |
| | | Gastrointestinal (0.192) | Inflammation (0.247) | |
| | | Nervous (0.178) | Cell Proliferation (0.233) | |
| 160 | 2108023 | Reproductive (0.750) | Cancer (0.500) | PSPORT1 |
| | | Nervous (0.250) | Inflammation (0.250) | |
| | | | Trauma (0.250) | |
| 161 | 2135746 | Nervous (0.321) | Cancer (0.500) | pINCY |
| | | Cardiovascular (0.214) | Inflammation (0.214) | |
| | | Reproductive (0.143) | Trauma (0.179) | |
| 162 | 2154810 | Cardiovascular (0.222) | Cancer (0.333) | pINCY |
| | | Developmental (0.222) | Cell Proliferation (0.333) | |
| | | Hematopoietic/Immune (0.222) | Inflammation (0.222) | |
| 163 | 2228991 | Hematopoietic/Immune (0.500) | Inflammation (0.333) | pINCY |
| | | Gastrointestinal (0.167) | Cancer (0.250) | |
| | | Reproductive (0.167) | Cell Proliferation (0.167) | |
| 164 | 2241206 | Cardiovascular (0.269) | Cancer (0.346) | pINCY |
| | | Gastrointestinal (0.154) | Cell Proliferation (0.346) | |
| | | Nervous (0.154) | Inflammation (0.308) | |
| 165 | 2259590 | Reproductive (0.375) | Cancer (0.500) | PSPORT1 |
| | | Urologic (0.250) | Cell Proliferation (0.250) | |
| | | Hematopoietic/Immune (0.125) | Inflammation (0.250) | |
| | | Developmental (0.125) | | |
| | | Endocrine (0.125) | | |
| 166 | 2307537 | Reproductive (0.241) | Cancer (0.414) | PSPORT1 |
| | | Gastrointestinal (0.138) | Cell Proliferation (0.241) | |
| | | Nervous (0.138) | Inflammation (0.241) | |
| 167 | 2440675 | Hematopoietic/Immune (0.600) | Inflammation (0.400) | pINCY |
| | | Cardiovascular (0.200) | Trauma (0.200) | |
| | | Reproductive (0.100) | Cell Proliferation (0.100) | |
| | | Gastrointestinal (0.100) | Cancer (0.100) | |
| 168 | 2463542 | Reproductive (0.333) | Cancer (0.542) | pINCY |
| | | Nervous (0.250) | Inflammation (0.292) | |
| | | Hematopoietic/Immune (0.125) | Trauma (0.125) | |
| 169 | 2486031 | Reproductive (0.333) | Cancer (0.333) | pINCY |
| | | Cardiovascular (0.167) | Cell Proliferation (0.250) | |
| | | Gastrointestinal (0.167) | | |
| | | Nervous (0.167) | | |
| 170 | 2493052 | Nervous (0.200) | Cancer (0.429) | pINCY |
| | | Gastrointestinal (0.171) | Cell Proliferation (0.343) | |
| | | Reproductive (0.171) | Inflammation (0.229) | |
| 171 | 2512074 | Hematopoietic/Immune (0.333) | Inflammation (0.500) | pINCY |
| | | Reproductive (0.333) | Cancer (0.417) | |
| | | Nervous (0.250) | Cell Proliferation (0.333) | |
| 172 | 2646274 | Gastrointestinal (0.207) | Cancer (0.379) | pINCY |
| | | Reproductive (0.207) | Inflammation (0.310) | |
| | | Developmental (0.138) | Cell Proliferation (0.207) | |
| 173 | 2672566 | Nervous (0.400) | Cancer (0.600) | pINCY |
| | | Gastrointestinal (0.200) | Cell Proliferation (0.100) | |
| | | Cardiovascular (0.100) | Inflammation (0.100) | |
| | | Hematopoietic/Immune (0.100) | Neurological (0.100) | |
| | | Reproductive (0.100) | | |
| 174 | 2689674 | Gastrointestinal (0.191) | Cancer (0.489) | pINCY |
| | | Reproductive (0.191) | Inflammation (0.191) | |
| | | Hematopoietic/Immune (0.170) | Cell Proliferation (0.149) | |
| 175 | 2703282 | Reproductive (0.409) | Cancer (0.409) | pINCY |
| | | Nervous (0.136) | Inflammation (0.386) | |
| | | Gastrointestinal (0.114) | Cell Proliferation (0.205) | |
| | | Hematopoietic/Immune (0.114) | | |
| 176 | 2738293 | Reproductive (0.333) | Cancer (0.416) | pINCY |
| | | Cardiovascular (0.167) | Cell Proliferation (0.167) | |
| | | Gastrointestinal (0.167) | Inflammation (0.167) | |
| | | | Trauma (0.167) | |
| 177 | 2772776 | Reproductive (0.232) | Cancer (0.500) | pINCY |
| | | Gastrointestinal (0.152) | Inflammation (0.205) | |
| | | Nervous (0.134) | Cell Proliferation (0.152) | |
| 178 | 2774476 | Gastrointestinal (0.712) | Trauma (0.429) | pINCY |
| | | Developmental (0.143) | Cancer (0.286) | |
| | | Nervous (0.143) | Cell Proliferation (0.286) | |
| 179 | 2804624 | Reproductive (0.252) | Cancer (0.480) | pINCY |
| | | Gastrointestinal (0.173) | Inflammation (0.236) | |
| | | Cardiovascular (0.150) | Trauma (0.134) | |
| 180 | 2848225 | Reproductive (0.385) | Cancer (0.385) | pINCY |
| | | Hematopoietic/Immune (0.231) | Trauma (0.308) | |
| | | Gastrointestinal (0.154) | Cell Proliferation (0.154) | |
| | | | Inflammation (0.154) | |
| 181 | 2882241 | Hematopoietic/Immune (0.259) | Cancer (0.519) | pINCY |
| | | Gastrointestinal (0.185) | Inflammation (0.333) | |
| | | Reproductive (0.185) | Cell Proliferation (0.148) | |
| 182 | 2939011 | Hematopoietic/Immune (0.263) | Cancer (0.316) | pINCY |
| | | Cardiovascular (0.158) | Cell Proliferation (0.316) | |
| | | Gastrointestinal (0.158) | Inflammation (0.316) | |
| | | Urologic (0.158) | | |
| | | Nervous (0.158) | | |
| 183 | 2947188 | Nervous (0.308) | Cancer (0.346) | pINCY |
| | | Gastrointestinal (0.154) | Inflammation (0.308) | |
| | | Reproductive (0.154) | Cell Proliferation (0.154) | |
| | | | Trauma (0.154) | |
| 184 | 3094001 | Reproductive (0.266) | Cancer (0.500) | pINCY |
| | | Gastrointestinal (0.160) | Inflammation (0.223) | |
| | | Nervous (0.160) | Cell Proliferation (0.160) | |
| 185 | 3110061 | Cardiovascular (0.333) | Inflammation (0.467) | pINCY |
| | | Hematopoietic/Immune (0.267) | Cancer (0.400) | |
| | | Nervous (0.200) | Cell Proliferation (0.267) | |
| | | Reproductive (0.200) | | |
| 186 | 3146614 | Reproductive (0.326) | Cancer (0.512) | pINCY |
| | | Nervous (0.209) | Inflammation (0.186) | |
| | | Gastrointestinal (0.163) | | |
| 187 | 3295381 | Hematopoietic/Immune (0.267) | Cancer (0.533) | pINCY |
| | | Musculoskeletal (0.200) | Inflammation (0.400) | |
| | | Reproductive (0.200) | | |
| 188 | 3364774 | Nervous (0.375) | Cancer (0.583) | pINCY |
| | | Gastrointestinal (0.208) | Cell Proliferation (0.250) | |
| | | Reproductive (0.167) | | |
| 189 | 3397777 | Reproductive (0.231) | Cancer (0.462) | pINCY |
| | | Cardiovascular (0.154) | Inflammation (0.385) | |
| | | Gastrointestinal (0.154) | | |
| | | Endocrine (0.154) | | |
| | | Hematopoietic/Immune (0.154) | | |
| 190 | 3403046 | Reproductive (0.500) | Cancer (0.500) | pINCY |
| | | Hematopoietic/Immune (0.250) | Inflammation (0.250) | |
| | | Nervous (0.250) | | |
| 191 | 3538506 | Reproductive (0.438) | Cancer (0.625) | pINCY |
| | | Gastrointestinal (0.188) | Trauma (0.250) | |
| | | Hematopoietic/Immune (0.188) | Cell Proliferation (0.188) | |
| | | Nervous (0.188) | | |
| 192 | 3575519 | Cardiovascular (0.455) | Trauma (0.455) | pINCY |
| | | Musculoskeletal (0.273) | Cancer (0.273) | |
| 193 | 3598694 | Nervous (0.247) | Cancer (0.575) | pINCY |
| | | Reproductive (0.247) | Cell Proliferation (0.233) | |
| | | | Inflammation (0.110) | |
| 194 | 3638819 | Reproductive (0.333) | Cancer (0.556) | pINCY |
| | | Nervous (0.222) | Inflammation (0.185) | |
| | | Gastrointestinal (0.111) | | |
| 195 | 3717139 | Hematopoietic/Immune (0.500) | Cancer (0.500) | pINCY |
| | | Reproductive (0.500) | Inflammation (0.500) | |
| 196 | 3892962 | Reproductive (0.455) | Cancer (0.909) | pINCY |
| | | Musculoskeletal (0.182) | Cell Proliferation (0.182) | |
| | | Nervous (0.182) | | |
| 197 | 4153521 | Nervous (0.281) | Cancer (0.453) | pINCY |
| | | Urologic (0.156) | Inflammation (0.203) | |
| | | Reproductive (0.141) | Cell Proliferation (0.156) | |
| 198 | 4585038 | Cardiovascular (0.261) | Cancer (0.261) | pINCY |
| | | Nervous (0.261) | Inflammation (0.261) | |
| | | Hematopoietic/Immune (0.174) | Cell Proliferation (0.130) | |
| | | | Trauma (0.130) | |
| 199 | 4674640 | Nervous (0.283) | Cancer (0.391) | pINCY |
| | | Reproductive (0.239) | Inflammation (0.304) | |
| | | Gastrointestinal (0.174) | Cell Proliferation (0.109) | |
| | | | Neurological (0.109) | |
| 200 | 4676066 | Reproductive (0.317) | Cancer (0.508) | pINCY |
| | | Cardiovascular (0.175) | Inflammation (0.159) | |
| | | Gastrointestinal (0.175) | Trauma (0.127) | |
| | | Nervous (0.175) | | |
| 201 | 4830687 | Reproductive (0.276) | Cancer (0.482) | pINCY |
| | | Gastrointestinal (0.147) | Cell Proliferation (0.218) | |
| | | Nervous (0.147) | Inflammation (0.194) | |
| 202 | 4880891 | Hematopoietic/Immune (0.190) | Cancer (0.381) | pINCY |
| | | Gastrointestinal (0.159) | Inflammation (0.333) | |
| | | Nervous (0.159) | Cell Proliferation (0.222) | |
| 203 | 4909754 | Reproductive (0.333) | Cancer (0.381) | pINCY |
| | | Hematopoietic/Immune (0.190) | Inflammation (0.333) | |
| | | Gastrointestinal (0.143) | Cell Proliferation (0.286) | |
| 204 | 4911931 | Nervous (0.219) | Cancer (0.375) | pINCY |
| | | Hematopoietic/Immune (0.188) | Cell Proliferation (0.312) | |
| | | Reproductive (0.125) | Inflammation (0.219) | |
| | | Cardiovascular (0.125) | | |
| 205 | 4920433 | Reproductive (1.000) | Inflammation (1.000) | pINCY |
| 206 | 5042113 | Gastrointestinal (0.206) | Cancer (0.413) | pINCY |
| | | Reproductive (0.159) | Inflammation (0.206) | |
| | | Nervous (0.159) | Cell Proliferation (0.190) | |
| 207 | 5083853 | Gastrointestinal (0.250) | Cancer (0.375) | pINCY |
| | | Hematopoietic/Immune (0.250) | Inflammation (0.375) | |
| | | Musculoskeletal (0.125) | Neurological (0.125) | |
| | | Reproductive (0.125) | | |
| | | Cardiovascular (0.125) | | |
| | | Nervous (0.125) | | |
| 208 | 5283981 | Reproductive (0.686) | Cancer (0.514) | pINCY |
| | | | Inflammation (0.171) | |
| | | | Cell proliferation (0.114) | |
| 209 | 5510549 | Hematopoietic/Immune (0.222) | Cancer (0.593) | pINCY |
| | | Reproductive (0.222) | Inflammation (0.148) | |
| | | Nervous (0.148) | Trauma (0.111) | |
| | | | Cell Proliferation (0.111) | |
| 210 | 5544862 | Endocrine (0.222) | Trauma (0.333) | pINCY |
| | | Nervous (0.222) | Inflammation (0.222) | |
| | | Reproductive (0.222) | Cell Proliferation (0.111) | |
| | | Gastrointestinal (0.111) | Neurological (0.111) | |
| | | Hematopoietic/Immune (0.111) | Cancer (0.111) | |
| 211 | 5573394 | Reproductive (0.194) | Cancer (0.463) | pINCY |
| | | Cardiovascular (0.149) | Inflammation (0.343) | |
| | | Hematopoietic/Immune (0.149) | Cell Proliferation (0.164) | |
| 212 | 5850840 | Nervous (0.295) | Cancer (0.416) | pINCY |
| | | Reproductive (0.268) | Inflammation (0.208) | |
| | | Cardiovascular (0.128) | Cell Proliferation (0.134) | |
| 213 | 5942936 | Nervous (0.444) | Cancer (0.556) | pINCY |
| | | Reproductive (0.333) | Inflammation (0.333) | |
| | | Cardiovascular (0.111) | Neurological (0.111) | |
| | | Musculoskeletal (0.111) | Trauma (0.111) | |
| 214 | 5951431 | Reproductive (0.317) | Cancer (0.518) | pINCY |
| | | Nervous (0.194) | Inflammation (0.194) | |
| | | Gastrointestinal (0.151) | Cell Proliferation (0.180) | |

TABLE 4

| Nucleotide | | | |
|---|---|---|---|
| SEQ ID NO: | | Library | Library Description |
| 108 | 095210 | PITUNOT01 | Library was constructed using RNA isolated from pituitary glands removed from a pool |
| | | | of 18 male and female Caucasian donors, 16 to 70 years old, who died from trauma. (RNA |
| | | | came from Clontech.) |
| 109 | 157953 | THP1PLB02 | Library was constructed by reamplification of a library made using RNA isolated from |
| | | | THP-1 cells cultured for 48 hours with 100 ng/ml phorbol ester (PMA), followed by a 4- |
| | | | hour culture in media containing 1 ug/ml LPS. THP-1 is a human promonocyte line |
| | | | derived from the peripheral blood of a 1-year-old male with acute monocytic leukemia. |
| | | | One million primary clones were amplified following phage packaging. |
| 110 | 159196 | ADENINB01 | Library was constructed using RNA isolated from the inflamed adenoid tissue of a 3- |
| | | | year-old child. (RNA came from Clontech.) |
| 111 | 343338 | THYMNOT02 | Library was constructed using RNA isolated from thymus tissue removed from a 3-year- |
| | | | old Caucasian male, who died from drowning. |
| 112 | 402386 | TMLR3DT01 | Library was constructed using RNA isolated from non-adherent and adherent peripheral |
| | | | blood mononuclear cells collected from two unrelated Caucasian male donors (25 and 29 |
| | | | years old). |
| 113 | 456487 | KERANOT01 | Library was constructed using RNA isolated from neonatal keratinocytes obtained from |
| | | | the leg skin of a spontaneously aborted black male. |
| 114 | 490256 | HNT2AGT01 | Library was constructed at Stratagene (STR937233), using RNA isolated from the hNT2 |
| | | | cell line derived from a human teratocarcinoma that exhibited properties |
| | | | characteristic of a committed neuronal precursor. Cells were treated with retinoic |
| | | | acid for 5 weeks, with mitotic inhibitors for two weeks and allowed to mature for an |
| | | | additional 4 weeks in conditioned medium. |
| 115 | 494740 | HNT2NOT01 | Library was constructed at Stratagene (STR937230), using RNA isolated from the hNT2 |
| | | | cell line (derived from a human teratocarcinoma that exhibited properties |
| | | | characteristic of a committed neuronal precursor). |
| 116 | 507475 | TMLR3DT01 | Library was constructed using RNA isolated from non-adherent and adherent peripheral |
| | | | blood mononuclear cells collected from two unrelated Caucasian male donors (25 and 29 |
| | | | years old). |
| 117 | 531581 | BRAINOT03 | Library was constructed using RNA isolated from brain tissue removed from a 26-year- |
| | | | old Caucasian male during cranioplasty and excision of a cerebral meningeal lesion. |
| | | | Pathology for the associated tumor tissue indicated a grade 4 oligoastrocytoma in the |
| | | | right fronto-parietal part of the brain. |
| 118 | 675190 | CRBLNOT01 | Library was constructed using RNA isolated from the cerebellum tissue of a 69-year-old |
| | | | Caucasian male who died from chronic obstructive pulmonary disease. Patient history |
| | | | included myocardial infarction, hypertension, and osteoarthritis. |
| 119 | 685434 | UTRSNOT02 | Library was constructed using RNA isolated from uterine tissue removed from a 34-year- |
| | | | old Caucasian female during a vaginal hysterectomy. Patient history included mitral |
| | | | valve disorder. Family history included stomach cancer, congenital heart anomaly, |
| | | | irritable bowel syndrome, ulcerative colitis, colon cancer, cerebrovascular disease, |
| | | | type II diabetes, and depression. |
| 120 | 788663 | PROSNOT05 | Library was constructed using RNA isolated from diseased prostate tissue removed from |
| | | | a 67-year-old Caucasian male during radical prostatectomy and lymph node biopsy. This |
| | | | library has been determined to contain some tumor cells. Pathology indicated |
| | | | adenofibromatous hyperplasia was present. Pathology for the associated tumor tissue |
| | | | indicated adenocarcinoma Gleason grade 3 + 3. Patient history included coronary artery |
| | | | disease, stomach ulcer, and osteoarthritis. Family history included congestive heart |
| | | | failure. |
| 121 | 870100 | LUNGAST01 | Library was constructed using RNA isolated from the lung tissue of a 17-year-old |
| | | | Caucasian male, who died from head trauma. Patient history included asthma. |
| 122 | 879500 | THYRNOT02 | Library was constructed using RNA isolated from the diseased thyroid tissue of a 16- |
| | | | year-old Caucasian female with Graves' disease (hyperthyroidism). |
| 123 | 975377 | MUSCNOT02 | Library was constructed using RNA isolated from the psoas muscle tissue of a 12-year- |
| | | | old Caucasian male. |
| 124 | 1208721 | BRSTNOT02 | Library was constructed using RNA isolated from diseased breast tissue removed from a |
| | | | 55-year-old Caucasian female during a unilateral extended simple mastectomy. |
| | | | Pathology indicated proliferative fibrocysytic changes characterized by apocrine |
| | | | metaplasia, sclerosing adenosis, cyst formation, and ductal hyperplasia without |
| | | | atypia. Pathology for the associated tumor tissue indicated an invasive grade 4 |
| | | | mammary adenocarcinoma. Patient history included atrial tachycardia and a benign |
| | | | neoplasm. Family history included cardiovascular and cerebrovascular disease. |
| 125 | 1234329 | LUNGFET03 | Library was constructed using RNA isolated from lung tissue removed from a Caucasian |
| | | | female fetus, who died at 20 weeks' gestation. |
| 126 | 1238747 | LUNGTUT02 | Library was constructed using RNA isolated from the metastatic lung tumor tissue of a |
| | | | 79-year-old Caucasian male. Pathology indicated a grade 4 carcinoma of the upper and |
| | | | lower left lobes. Patient history included a benign prostate neoplasm, |
| | | | atherosclerosis, and tobacco use. |
| 127 | 1265980 | BRAINOT09 | Library was constructed using RNA isolated from brain tissue removed from a Caucasian |
| | | | male fetus, who died at 23 weeks' gestation. |
| 128 | 1297333 | BRSTNOT07 | Library was constructed using RNA isolated from diseased breast tissue removed from a |
| | | | 43-year-old Caucasian female during a unilateral extended simple mastectomy. |
| | | | Pathology indicated mildly proliferative fibrocystic changes with epithelial |
| | | | hyperplasia, papillomatosis, and duct ectasia. Pathology for the associated tumor |
| | | | tissue indicated invasive grade 4, nuclear grade 3 mammary adenocarcinoma with |
| | | | extensive comedo necrosis. Family history included epilepsy, cardiovascular disease, |
| | | | and type II diabetes. |
| 129 | 1312824 | BLADTUT02 | Library was constructed using RNA isolated from bladder tumor tissue removed from an |
| | | | 80-year-old Caucasian female during a radical cystectomy and lymph node excision. |
| | | | Pathology indicated grade 3 invasive transitional cell carcinoma. Family history |
| | | | included acute renal failure, osteoarthritis, and atherosclerosis. |
| 130 | 1359294 | LUNGNOT12 | Library was constructed using RNA isolated from lung tissue removed from a 78-year-old |
| | | | Caucasian male during a segmental lung resection and regional lymph node resection. |
| | | | Pathology indicated fibrosis pleura was puckered, but not invaded. Pathology for the |
| | | | associated tumor tissue indicated an invasive pulmonary grade 3 adenocarcinoma. |
| | | | Patient history included cerebrovascular disease, arteriosclerotic coronary artery |
| | | | disease, thrombophlebitis, chronic obstructive pulmonary disease, and asthma. Family |
| | | | history included intracranial hematoma, cerebrovascular disease, arteriosclerotic |
| | | | coronary artery disease, and type I diabetes. |
| 131 | 1377380 | LUNGNOT10 | Library was constructed using RNA isolated from the lung tissue of a Caucasian male |
| | | | fetus, who died at 23 weeks' gestation. |
| 132 | 1383473 | BRAITUT08 | Library was constructed using RNA isolated from brain tumor tissue removed from the |
| | | | left frontal lobe of a 47-year-old Caucasian male during excision of cerebral |
| | | | meningeal tissue. Pathology indicated grade 4 fibrillary astrocytoma with focal |
| | | | tumoral radionecrosis. Patient history included cerebrovascular disease, deficiency |
| | | | anemia, hyperlipidemia, epilepsy, and tobacco use. Family history included |
| | | | cerebrovascular disease and a malignant prostate neoplasm. |
| 133 | 1388860 | EOSINOT01 | Library was constructed using RNA isolated from microscopically normal eosinophils |
| | | | from 31 non-allergic donors. |
| 134 | 1395322 | THYRNOT03 | Library was constructed using RNA isolated from thyroid tissue removed from the left |
| | | | thyroid of a 28-year-old Caucasian female during a complete thyroidectomy. Pathology |
| | | | indicated a small nodule of adenomatous hyperplasia present in the left thyroid. |
| | | | Pathology for the associated tumor tissue indicated dominant follicular adenoma, |
| | | | forming a well-encapsulated mass in the left thyroid. |
| 135 | 1419370 | KIDNNOT09 | Library was constructed using RNA isolated from the kidney tissue of a Caucasian male |
| | | | fetus, who died at 23 weeks' gestation. |
| 136 | 1429773 | SINTBST01 | Library was constructed using RNA isolated from ileum tissue obtained from an 18-year- |
| | | | old Caucasian female during bowel anastomosis. Pathology indicated Crohn's disease of |
| | | | the ileum, involving 15 cm of the small bowel. Family history included |
| | | | cerebrovascular disease and atherosclerotic coronary artery disease. |
| 137 | 1470820 | PANCTUT02 | Library was constructed using RNA isolated from pancreatic tumor tissue removed from a |
| | | | 45-year-old Caucasian female during radical pancreaticoduodenectomy. Pathology |
| | | | indicated a grade 4 anaplastic carcinoma. Family history included benign hypertension, |
| | | | hyperlipidemia and atherosclerotic coronary artery disease. |
| 138 | 1483455 | CORPNOT02 | Library was constructed using RNA isolated from diseased corpus callosum tissue |
| | | | removed from the brain of a 74-year-old Caucasian male who died from Alzheimer's |
| | | | disease. |
| 139 | 1527064 | UCMCL5T01 | Library was constructed using RNA isolated from mononuclear cells obtained from the |
| | | | umbilical cord blood of 12 individuals. The cells were cultured for 12 days with IL-5 |
| | | | before RNA was obtained from the pooled lysates. |
| 140 | 1557491 | BLADTUT04 | Library was constructed using RNA isolated from bladder tumor tissue removed from a |
| | | | 60-year-old Caucasian male during a radical cystectomy, prostatectomy, and vasectomy. |
| | | | Pathology indicated grade 3 transitional cell carcinoma in the left bladder wall. |
| | | | Carcinoma in-situ was identified in the dome and trigone. Patient history included |
| | | | tobacco use. Family history included type I diabetes, malignant neoplasm of the |
| | | | stomach, atherosclerotic coronary artery disease, and acute myocardial infarction. |
| 141 | 1576862 | LNODNOT03 | Library was constructed using RNA isolated from lymph node tissue obtained from a 67- |
| | | | year-old Caucasian male during a segmental lung resection and bronchoscopy. This |
| | | | tissue was extensively necrotic with 10% viable tumor. Pathology for the associated |
| | | | tumor tissue indicated invasive grade 3-4 squamous cell carcinoma. Patient history |
| | | | included hemangioma. Family history included atherosclerotic coronary artery disease, |
| | | | benign hypertension, and congestive heart failure. |
| 142 | 1609731 | COLNTUT06 | Library was constructed using RNA isolated from colon tumor tissue obtained from a 45- |
| | | | year-old Caucasian female during a total colectomy and total abdominal hysterectomy. |
| | | | Pathology indicated invasive grade 2 colonic adenocarcinoma forming a cecal mass. |
| | | | Patient history included benign neoplasms of the rectum and anus, multiple sclerosis |
| | | | and mitral valve disorder. Previous surgeries included a polypectomy. Family history |
| | | | included type I diabetes, cerebrovascular disease, malignant skin neoplasm, |
| | | | hypertension, atherosclerotic coronary artery disease and malignant neoplasm of the |
| | | | colon. |
| 143 | 1674538 | BLADNOT05 | Library was constructed using RNA isolated from bladder tissue removed from a 60-year- |
| | | | old Caucasian male during a radical cystectomy, prostatectomy, and vasectomy. |
| | | | Pathology for the associated tumor tissue indicated grade 3 transitional cell |
| | | | carcinoma. Carcinoma in-situ was identified in the dome and trigone. Patient history |
| | | | included tobacco use. |
| 144 | 1675287 | BLADNOT05 | Library was constructed using RNA isolated from bladder tissue removed from a 60-year- |
| | | | old Caucasian male during a radical cystectomy, prostatectomy, and vasectomy. |
| | | | Pathology for the associated tumor tissue indicated grade 3 transitional cell |
| | | | carcinoma. Carcinoma in-situ was identified in the dome and trigone. Patient history |
| | | | included tobacco use. |
| 145 | 1693903 | COLNNOT23 | Library was constructed using RNA isolated from diseased colon tissue removed from a |
| | | | 16-year-old Caucasian male during a total colectomy with abdominal/perineal resection. |
| | | | Pathology indicated gastritis and pancolonitis consistent with the acute phase of |
| | | | ulcerative colitis. Inflammation was more severe in the transverse colon, with |
| | | | inflammation confined to the mucosa. There was only mild involvement of the ascending |
| | | | and sigmoid colon. Family history included irritable bowel syndrome. |
| 146 | 1702962 | DUODNOT02 | Library was constructed using RNA isolated from duodenal tissue of an 8-year-old |
| | | | Caucasian female, who died from head trauma. Serology was positive for cytomegalovirus |
| | | | (CMV). |
| 147 | 1712916 | PROSNOT16 | Library was constructed using RNA isolated from diseased prostate tissue removed from |
| | | | a 68-year-old Caucasian male during a radical prostatectomy. Pathology indicated |
| | | | adenofibromatous hyperplasia. Pathology for the associated tumor tissue indicated an |
| | | | adenocarcinoma (Gleason grade 3 + 4). The patient presented with elevated prostate |
| | | | specific antigen (PSA). During this hospitalization, the patient was diagnosed with |
| | | | myasthenia gravis. Patient history included osteoarthritis, and type II diabetes. |
| | | | Family history included benign hypertension, acute myocardial infarction, |
| | | | hyperlipidemia, and arteriosclerotic coronary artery disease. |
| 148 | 1748313 | STOMTUT02 | Library was constructed using RNA isolated from stomach tumor tissue obtained from a |
| | | | 68-year-old Caucasian female during a partial gastrectomy. Pathology indicated a |
| | | | malignant lymphoma of diffuse large-cell type. Previous surgeries included |
| | | | cholecystectomy. Patient history included thalassemia. Family history included acute |
| | | | leukemia, malignant esophagus and stomach neoplasms, and atherosclerotic coronary |
| | | | artery disease. |
| 149 | 1754833 | LIVRTUT01 | Library was constructed using RNA isolated from liver tumor tissue removed from a 51- |
| | | | year-old Caucasian female during a hepatic lobectomy. Pathology indicated metastatic |
| | | | grade 3 adenocarcinoma consistent with colon cancer. Family history included a |
| | | | malignant neoplasm of the liver. |
| 150 | 1798701 | COLNNOT27 | Library was constructed using RNA isolated from diseased cecal tissue removed from a |
| | | | 31-year-old Caucasian male during a total intra-abdominal colectomy, appendectomy, and |
| | | | permanent ileostomy. Pathology indicated severe active Crohn's disease involving the |
| | | | colon from the cecum to the rectum. There were deep rake-like ulcerations that spared |
| | | | the intervening mucosa. The ulcers extended into the muscularis, and there was |
| | | | transmural inflammation. Patient history included an irritable colon. Previous |
| | | | surgeries included a colonscopy. |
| 151 | 1842496 | COLNNOT07 | Library was constructed using RNA isolated from colon tissue removed from a 60-year- |
| | | | old Caucasian male during a left hemicolectomy. |
| 152 | 1868613 | SKINBIT01 | Library was constructed using RNA isolated from diseased skin tissue of the left lower |
| | | | leg. Patient history included erythema nodosum of the left lower leg. |
| 153 | 1870609 | SKINBIT01 | Library was constructed using RNA isolated from diseased skin tissue of the left lower |
| | | | leg. Patient history included erythema nodosum of the left lower leg. |
| 154 | 1871961 | LEUKNOT02 | Library was constructed using RNA isolated from white blood cells of a 45-year-old |
| | | | female with blood type O+. The donor tested positive for cytomegalovirus (CMV). |
| 155 | 1876258 | LEUKNOT02 | Library was constructed using RNA isolated from white blood cells of a 45-year-old |
| | | | female with blood type O+. The donor tested positive for cytomegalovirus (CMV). |
| 156 | 1929822 | COLNTUT03 | Library was constructed using RNA isolated from colon tumor tissue obtained from the |
| | | | sigmoid colon of a 62-year-old Caucasian male during a sigmoidectomy and permanent |
| | | | colostomy. Pathology indicated invasive grade 2 adenocarcinoma. One lymph node |
| | | | contained metastasis with extranodal extension. Patient history included |
| | | | hyperlipidemia, cataract disorder, and dermatitis. Family history included benign |
| | | | hypertension, atherosclerotic coronary artery disease, hyperlipidemia, breast cancer |
| | | | and prostate cancer. |
| 157 | 1970095 | UCMCL5T01 | Library was constructed using RNA isolated from mononuclear cells obtained from the |
| | | | umbilical cord blood of 12 individuals. The cells were cultured for 12 days with IL-5 |
| | | | before RNA was obtained from the pooled lysates. |
| 158 | 1975473 | PANCTUT02 | Library was constructed using RNA isolated from pancreatic tumor tissue removed from a |
| | | | 45-year-old Caucasian female during radical pancreaticoduodenectomy. Pathology |
| | | | indicated a grade 4 anaplastic carcinoma. Family history included benign hypertension, |
| | | | hyperlipidemia and atherosclerotic coronary artery disease. |
| 159 | 1976527 | PANCTUT02 | Library was constructed using RNA isolated from pancreatic tumor tissue removed from a |
| | | | 45-year-old Caucasian female during radical pancreaticoduodenectomy. Pathology |
| | | | indicated a grade 4 anaplastic carcinoma. Family history included benign hypertension, |
| | | | hyperlipidemia and atherosclerotic coronary artery disease. |
| 160 | 2108023 | BRAITUT03 | Library was constructed using RNA isolated from brain tumor tissue removed from the |
| | | | left frontal lobe a 17-year-old Caucasian female during excision of a cerebral |
| | | | meningeal lesion. Pathology indicated a grade 4 fibrillary giant and small-cell |
| | | | astrocytoma. Family history included benign hypertension and cerebrovascular disease. |
| 161 | 2135746 | ENDCNOT01 | Library was constructed using RNA isolated from endothelial cells removed from the |
| | | | coronary artery of a 58-year-old Hispanic male. |
| 162 | 2154810 | BRAINOT09 | Library was constructed using RNA isolated from brain tissue removed from a Caucasian |
| | | | male fetus, who died at 23 weeks' gestation. |
| 163 | 2228991 | PROSNOT16 | Library was constructed using RNA isolated from diseased prostate tissue removed from |
| | | | a 68-year-old Caucasian male during a radical prostatectomy. Pathology indicated |
| | | | adenofibromatous hyperplasia. Pathology for the associated tumor tissue indicated an |
| | | | adenocarcinoma (Gleason grade 3 + 4). The patient presented with elevated prostate |
| | | | specific antigen (PSA). During this hospitalization, the patient was diagnosed with |
| | | | myasthenia gravis. Patient history included osteoarthritis, and type II diabetes. |
| | | | Family history included benign hypertension, acute myocardial infarction, |
| | | | hyperlipidemia, and arteriosclerotic coronary artery disease. |
| 164 | 2241206 | PANCTUT02 | Library was constructed using RNA isolated from pancreatic tumor tissue removed from a |
| | | | 45-year-old Caucasian female during radical pancreaticoduodenectomy. Pathology |
| | | | indicated a grade 4 anaplastic carcinoma. Family history included benign hypertension, |
| | | | hyperlipidemia and atherosclerotic coronary artery disease. |
| 165 | 2259590 | OVARTUT01 | Library was constructed using RNA isolated from ovarian tumor tissue removed from a |
| | | | 43-year-old Caucasian female during removal of the fallopian tubes and ovaries. |
| | | | Pathology indicated grade 2 mucinous cystadenocarcinoma involving the entire left |
| | | | ovary. Patient history included mitral valve disorder, pneumonia, and viral |
| | | | hepatitis. Family history included atherosclerotic coronary artery disease, |
| | | | pancreatic cancer, stress reaction, cerebrovascular disease, breast cancer, and |
| | | | uterine cancer. |
| 166 | 2307537 | NGANNOT01 | Library was constructed using RNA isolated from tumorous neuroganglion tissue removed |
| | | | from a 9-year-old Caucasian male during a soft tissue excision of the chest wall. |
| | | | Pathology indicated a ganglioneuroma. Family history included asthma. |
| 167 | 2440675 | EOSITXT01 | Library was constructed using RNA isolated from eosinophils stimulated with IL-5. |
| 168 | 2463542 | THYRNOT03 | Library was constructed isolated from the diseased left thyroid tissue removed from a |
| | | | 13-year-old Caucasian female during a complete thyroidectomy. Pathology indicated |
| | | | lymphocytic thyroiditis. |
| 169 | 2486031 | CONUTUT01 | Library was constructed using RNA isolated from sigmoid mesentery tumor tissue |
| | | | obtained from a 61-year-old female during a total abdominal hysterectomy and bilateral |
| | | | salpingo-oophorectomy with regional lymph node excision. Pathology indicated a |
| | | | metastatic grade 4 malignant mixed mullerian tumor present in the sigmoid mesentery at |
| | | | two sites. |
| 170 | 2493052 | ADRETUT05 | Library was constructed using RNA isolated from adrenal tumor tissue removed from a |
| | | | 52-year-old Caucasian female during a unilateral adrenalectomy. Pathology indicated a |
| | | | pheochromocytoma. |
| 171 | 2512074 | CONUTUT01 | Library was constructed using RNA isolated from sigmoid mesentery tumor tissue |
| | | | obtained from a 61-year-old female during a total abdominal hysterectomy and bilateral |
| | | | salpingo-oophorectomy with regional lymph node excision. Pathology indicated a |
| | | | metastatic grade 4 malignant mixed mullerian tumor present in the sigmoid mesentery at |
| | | | two sites. |
| 172 | 2646274 | LUNGTUT11 | Library was constructed using RNA isolated from lung tumor tissue removed from the |
| | | | right lower lobe of a 57-year-old Caucasian male during a segmental lung resection. |
| | | | Pathology indicated an infiltrating grade 4 squamous cell carcinoma. Multiple |
| | | | intrapulmonary peribronchial lymph nodes showed metastatic squamous cell carcinoma. |
| | | | Patient history included a benign brain neoplasm and tobacco abuse. Family history |
| | | | included spinal cord cancer, type II diabetes, cerebrovascular disease, and malignant |
| | | | prostate neoplasm. |
| 173 | 2672566 | KIDNNOT19 | Library was constructed using RNA isolated from kidney tissue removed a 65-year-old |
| | | | Caucasian male during an exploratory laparotomy and nephroureterectomy. Patient |
| | | | history included malignant melanoma of the abdominal skin, benign neoplasm of colon, |
| | | | cerebrovascular disease, and umbilical hernia. Family history included |
| | | | cerebrovascular disease, prostate cancer, myocardial infarction, and atherosclerotic |
| | | | coronary artery disease. |
| 174 | 2689674 | LUNGNOT23 | Library was constructed using RNA isolated from left lobe lung tissue removed from a |
| | | | 58-year-old Caucasian male. Patient history included soft tissue cancer, secondary |
| | | | cancer of the lung, prostate cancer, and an acute duodenal ulcer with hemorrhage. |
| | | | Family history included prostate cancer, breast cancer, and acute leukemia. |
| 175 | 2703282 | OVARTUT10 | Library was constructed using RNA isolated from ovarian tumor tissue removed from the |
| | | | left ovary of a 58-year-old Caucasian female during a total abdominal hysterectomy, |
| | | | removal of a solitary ovary, and repair of inguinal hernia. Pathology indicated a |
| | | | metastatic grade 3 adenocarcinoma of colonic origin, forming a partially cystic and |
| | | | necrotic tumor mass in the left ovary, and an adenocarcinoma of colonic origin, |
| | | | forming a nodule in the left mesovarium. A single intramural leiomyoma was identified |
| | | | in the myometrium. The cervix showed mild chronic cystic cervicitis. Patient history |
| | | | included benign hypertension, follicular cyst of the ovary, colon cancer, benign colon |
| | | | neoplasm, and osteoarthritis. Family history included emphysema, myocardial |
| | | | infarction, atherosclerotic coronary artery disease, benign hypertension, and |
| | | | hyperlipidemia. |
| 176 | 2738293 | OVARNOT09 | Library was constructed using RNA isolated from ovarian tissue removed from a 28-year- |
| | | | old Caucasian female during a vaginal hysterectomy and removal of the fallopian tubes |
| | | | and ovaries. Pathology indicated multiple follicular cysts ranging in size from 0.4 |
| | | | to 1.5 cm in the right and left ovaries, chronic cervicitis and squamous metaplasia of |
| | | | the cervix, and endometrium in weakly proliferative phase. Family history included |
| | | | benign hypertension, hyperlipidemia, and atherosclerotic coronary artery disease. |
| 177 | 2772776 | PANCNOT15 | Library was constructed using RNA isolated from diseased pancreatic tissue removed |
| | | | from a 15-year-old Caucasian male during an exploratory laparotomy with distal |
| | | | pancreatectomy and total splenectomy. Pathology indicated islet cell hyperplasia. |
| | | | Family history included prostate cancer and cardiovacular disease. |
| 178 | 2774476 | PANCNOT15 | Library was constructed using RNA isolated from diseased pancreatic tissue removed |
| | | | from a 15-year-old Caucasian male during an exploratory laparotomy with distal |
| | | | pancreatectomy and total splenectomy. Pathology indicated islet cell hyperplasia. |
| | | | Family history included prostate cancer and cardiovacular disease. |
| 179 | 2804624 | BLADTUT08 | Library was constructed using RNA isolated from bladder tumor tissue removed from a |
| | | | 72-year-old Caucasian male during a radical cystectomy and prostatectomy. Pathology |
| | | | indicated an invasive grade 3 (of 3) transitional cell carcinoma in the right bladder |
| | | | base. Patient history included pure hypercholesterolemia and tobacco abuse. Family |
| | | | history included cerebrovascular disease, brain cancer, and myocardial infarction. |
| 180 | 2848225 | BRSTTUT13 | Library was constructed using RNA isolated from breast tumor tissue removed from the |
| | | | right breast of a 46-year-old Caucasian female during a unilateral extended simple |
| | | | mastectomy with breast reconstruction. Pathology indicated an invasive grade 3 |
| | | | adenocarcinoma, ductal type with apocrine features and greater than 50% intraductal |
| | | | component. Patient history included breast cancer. |
| 181 | 2882241 | UTRSTUT05 | Library was constructed using RNA isolated from uterine tumor tissue removed from a |
| | | | 41-year-old Caucasian female during a vaginal hysterectomy with dilation and |
| | | | curettage. Pathology indicated uterine leiomyoma. The endometrium was secretory and |
| | | | contained fragments of endometrial polyps. Benign endo- and ectocervical mucosa were |
| | | | identified in the endocervix. Patient history included a ventral hernia and a benign |
| | | | ovarian neoplasm. |
| 182 | 2939011 | THYMFET02 | Library was constructed using RNA isolated from thymus tissue removed from a Caucasian |
| | | | female fetus, who died at 17 weeks' gestation from anencephalus. |
| 183 | 2947188 | BRAITUT23 | Library was constructed using RNA isolated from left posterior brain tumor tissue |
| | | | removed from a 36-year-old male during a cerebral meninges lesion excision. Pathology |
| | | | indicated meningioma. Family history included malignant skin melanoma, |
| | | | atherosclerotic coronary artery disease, repair of unspecified vessel, hyperlipidemia, |
| | | | Huntington's chorea, and rheumatoid arthritis. |
| 184 | 3094001 | BRSTNOT19 | Library was constructed using RNA isolated from breast tissue removed from a 67-year- |
| | | | old Caucasian female during a unilateral extended simple mastectomy. Patient history |
| | | | included depressive disorder and benign large bowel neoplasm. Family history included |
| | | | cerebrovascular disease, benign hypertension, congestive heart failure, and lung |
| | | | cancer. |
| 185 | 3110061 | BRSTNOT19 | Library was constructed using RNA isolated from breast tissue removed from a 67-year- |
| | | | old Caucasian female during a unilateral extended simple mastectomy. Patient history |
| | | | included depressive disorder, benign large bowel neoplasm, and hemorrhoids. Family |
| | | | history included cerebrovascular and cardiovascular disease and lung cancer. |
| 186 | 3146614 | BRSTTUT15 | Library was constructed using RNA isolated from breast tumor tissue removed from a 46- |
| | | | year-old Caucasian female during a unilateral extended simple mastectomy. Pathology |
| | | | indicated invasive grade 3, nuclear grade 2 adenocarcinoma, ductal type. An |
| | | | intraductal carcinoma component, non-comedo, comprised approximately 50% of the |
| | | | neoplasm, including the lactiferous ducts. Angiolymphatic involvement was present. |
| | | | Metastatic adenocarcinoma was present in 7 of 10 axillary lymph nodes. The largest |
| | | | nodal metastasis measured 3 cm, and focal extracapsular extension was identified. |
| | | | Family history included atherosclerotic coronary artery disease, type II diabetes, |
| | | | cerebrovascular disease, and depression. |
| 187 | 3295381 | PENCNOT06 | Library was constructed using RNA isolated from penis corpora cavernosa tissue removed |
| | | | from a 3-year-old Black male. |
| 188 | 3364774 | TLYJINT01 | Library was constructed using RNA isolated from a Jurkat cell line derived from the T |
| | | | cells of a male. The cells were treated for 18 hours with 50 ng/ml. phorbol ester (PMA) |
| | | | and 1 micromolar calcium ionophore. Patient history included acute T-cell leukemia. |
| 189 | 3397777 | PROSBPT02 | Library was constructed using RNA isolated from diseased prostate tissue removed from |
| | | | a 65-year-old Caucasian male during a radical prostatectomy. Pathology indicated |
| | | | benign prostatic hyperplasia (BPH). One (of 7) right pelvic lymph nodes was positive |
| | | | for metastatic adenocarcinoma. The patient presented with induration and elevated |
| | | | prostate specific antigen (PSA). Patient history included a benign neoplasm of the |
| | | | large bowel and benign hypertension. |
| 190 | 3403046 | ESOGNOT03 | Library was constructed using RNA isolated from esophageal tissue obtained from a 53- |
| | | | year-old Caucasian male during a partial esophagectomy, proximal gastrectomy, and |
| | | | regional lymph node biopsy. Patient history included membranous nephritis, |
| | | | hyperlipidemia, benign hypertension, and anxiety state. Previous surgeries included |
| | | | an adenotonsillectomy. Family history included cirrhosis, abdominal aortic aneurysm |
| | | | rupture, breast cancer, myocardial infarction, and atherosclerotic coronary artery |
| | | | disease. |
| 191 | 3538506 | SEMVNOT04 | Library was constructed using RNA isolated from seminal vesicle tissue removed from a |
| | | | 61-year-old Caucasian male during a radical prostatectomy. Pathology for the |
| | | | associated tumor tissue indicated adenocarcinoma, Gleason grade 3 + 3. The patient |
| | | | presented with induration, hyperplasia of the prostate, and elevated prostate specific |
| | | | antigen. Patient history included renal failure, osteoarthritis, left renal artery |
| | | | stenosis, thrombocytopenia, hyperlipidemia, and hepatitis C (carrier). Family history |
| | | | included benign hypertension. |
| 192 | 3575519 | BRONNOT01 | Library was constructed using RNA isolated from bronchial tissue removed from a 15- |
| | | | year-old Caucasian male. |
| 193 | 3598694 | FIBPNOT01 | Library was constructed using RNA isolated from fibroblasts of the prostate stroma |
| | | | removed from a male fetus, who died after 26 weeks' gestation. |
| 194 | 3638819 | LUNGNOT30 | Library was constructed using RNA isolated from lung tissue removed from a Caucasian |
| | | | male fetus, who died from Patau's syndrome (trisomy 13) at 20-weeks' gestation. |
| 195 | 3717139 | PENCNOT10 | Library was constructed using RNA isolated from penis left corpora cavernosa tissue |
| | | | removed from a male. |
| 196 | 3892962 | BRSTTUT16 | Library was constructed using RNA isolated from breast tumor tissue removed from a 43- |
| | | | year-old Caucasian female during a unilateral extended simple mastectomy. Pathology |
| | | | indicated recurrent grade 4, nuclear grade 3, ductal carcinoma. Angiolymphatic space |
| | | | invasion was identified. Left breast needle biopsy indicated grade 4 ductal |
| | | | adenocarcinoma. Paraffin embedded tissue was estrogen positive. Patient history |
| | | | included breast cancer and deficiency anemia. Family history included cervical |
| | | | cancer. |
| 197 | 4153521 | MUSLTMT01 | Library was constructed using RNA isolated from glossal muscle tissue removed from a |
| | | | 41-year-old Caucasian female during partial glossectomy. Pathology for the matched |
| | | | tumor tissue indicated invasive grade 3, squamous cell carcinoma forming an ulcerated |
| | | | mass of the tongue. The tumor infiltrated superficially into muscle. One high lymph |
| | | | node contained a necrotizing granuloma. The patient presented with a complicated open |
| | | | wound of the tongue. Patient history included obesity, unspecified nasal and sinus |
| | | | disease, and normal delivery. Patient medications included Premarin, Hydrocodone, and |
| | | | Equate nasal spray. Family history included benign hypertension, atherosclerotic |
| | | | coronary artery disease, upper lobe lung cancer, type II diabetes, hyperlipidemia, and |
| | | | cirrhosis of the liver. |
| 198 | 4585038 | OVARNOT13 | Library was constructed using RNA isolated from left ovary tissue removed from a 47- |
| | | | year-old Caucasian female during a vaginal hysterectomy with bilateral salpingo- |
| | | | oophorectomy, and dilation and curettage. Pathology for the associated tumor tissue |
| | | | indicated a single intramural leiomyoma. The endometrium was in the secretory phase. |
| | | | The patient presented with metrorrhagia. Patient history included hyperlipidemia and |
| | | | benign hypertension. Family history included colon cancer, benign hypertension, |
| | | | atherosclerotic coronary artery disease, and breast cancer. |
| 199 | 4674640 | NOSEDIT02 | Library was constructed using RNA isolated from nasal polyp tissue. |
| 200 | 4676066 | NOSEDIT02 | Library was constructed using RNA isolated from nasal polyp tissue. |
| 201 | 4830687 | BRAVTXT03 | Library was constructed using RNA isolated from treated astrocytes removed from the |
| | | | brain of a female fetus who died after 22 weeks' gestation. The cells were treated |
| | | | with tumor necrosis factor-alpha (TNF) and interleukin 1 (IL-1), 10 ng/ml each for 24 |
| | | | hours. |
| 202 | 4880891 | UTRMTMT01 | Library was constructed using RNA isolated from myometrial tissue removed from a 45- |
| | | | year-old Caucasian female during vaginal hysterectomy and bilateral salpingo- |
| | | | oophorectomy. Pathology for the matched tumor tissue indicated multiple (23) |
| | | | subserosal, intramural, and submucosal leiomyomata. The endometrium was in |
| | | | proliferative phase. The right ovary contained an old corpus luteum. The patient |
| | | | presented with stress incontinence. Patient history included normal delivery. Patient |
| | | | medications included Motrin, iron sulfate, Premarin, prednisone, Tylenol #3, and |
| | | | Colace. Family history included cerebrovascular disease, depression, and |
| | | | atherosclerotic coronary artery disease. |
| 203 | 4909754 | THYMDIT01 | Library was constructed using RNA isolated from diseased thymus tissue removed from a |
| | | | 16-year-old Caucasian female during a total excision of thymus and regional lymph node |
| | | | excision. Pathology indicated thymic follicular hyperplasia. The right lateral thymus |
| | | | showed reactive lymph nodes. A single reactive lymph node was also identified at the |
| | | | inferior thymus margin. The patient presented with myasthenia gravis, malaise, |
| | | | fatigue, dysphagia, severe muscle weakness, and prominent eyes. Patient history |
| | | | included frozen face muscles. Family history included depression, hepatitis B, |
| | | | myocardial infarction, atherosclerotic coronary artery disease, leukemia, multiple |
| | | | sclerosis, and lupus. |
| 204 | 4911931 | THYMDIT01 | Library was constructed using RNA isolated from diseased thymus tissue removed from a |
| | | | 16-year-old Caucasian female during a total excision of thymus and regional lymph node |
| | | | excision. Pathology indicated thymic follicular hyperplasia. The right lateral thymus |
| | | | showed reactive lymph nodes. A single reactive lymph node was also identified at the |
| | | | inferior thymus margin. The patient presented with myasthenia gravis, malaise, |
| | | | fatigue, dysphagia, severe muscle weakness, and prominent eyes. Patient history |
| | | | included frozen face muscles. Family history included depression, hepatitis B, |
| | | | myocardial infarction, atherosclerotic coronary artery disease, leukemia, multiple |
| | | | sclerosis, and lupus. |
| 205 | 4920433 | TESTNOT11 | Library was constructed using RNA isolated from testicular tissue removed from a 16- |
| | | | year-old Caucasian male who died from hanging. |
| 206 | 5042113 | COLHTUT01 | Library was constructed using RNA isolated from colon tumor tissue removed from the |
| | | | hepatic flexure of a 55-year-old Caucasian male during right hemicolectomy, incidental |
| | | | appendectomy, and permanent colostomy. Pathology indicated invasive grade 3 |
| | | | adenocarcinoma. Patient history included benign hypertension, anxiety, abnormal blood |
| | | | chemistry, blepharitis, heart block, osteoporosis, acne, and hyperplasia of prostate. |
| | | | Family history included prostate cancer, acute myocardial infarction, stroke, and |
| | | | atherosclerotic coronary artery disease. |
| 207 | 5083853 | LNOGTUT01 | Library was constructed using RNA isolated from gastric lymph node tumor tissue |
| | | | removed from a 61-year-old Caucasian male during proximal gastrectomy and partial |
| | | | esophagectomy. Pathology indicated invasive grade 3 adenocarcinoma forming an |
| | | | ulcerated, plaque-like mass situated at the lower esophagus just proximal to the |
| | | | gastroesophageal junction, with partial involvement of cardiac mucosa. Metastatic |
| | | | adenocarcinoma was identified in 2 of 3 paraesophageal and 9 of 14 paragastric lymph |
| | | | nodes with perinodal extension to form grossly matted nodes. The paraesophageal lymph |
| | | | node contained metastatic grade 3 adenocarcinoma with perinodal extension. Tissue from |
| | | | the mesentery showed dense fibrosis with chronic inflammation and focal calcification. |
| | | | Patient history included a benign colon neoplasm and hyperlipidemia. Family history |
| | | | included type II diabetes, accessory sinus cancer, atherosclerotic coronary artery |
| | | | disease, and acute myocardial infarction. |
| 208 | 5283981 | TESTNON04 | This normalized testis tissue library was constructed from 6.48 million independent |
| | | | clones from a pool of two testicular libraries. Starting RNA was made from testicular |
| | | | tissue removed from a 16-year-old Caucasian male who died from hanging. The library |
| | | | was normalized in two rounds using conditions adapted from Soares et al., PNAS (1994) |
| | | | 91: 9228 and Bonaldo et al. except that a significantly longer (48-hours/round) |
| | | | reannealing hybridization was used. |
| 209 | 5510549 | BRADDIR01 | Library was constructed using RNA isolated from diseased choroid plexus tissue of the |
| | | | lateral ventricle removed from the brain of a 57-year-old Caucasian male, who died |
| | | | from a cerebrovascular accident. Patient history included Huntington's disease and |
| | | | emphysema. |
| 210 | 5544862 | BRADDIR01 | Library was constructed using RNA isolated from diseased choroid plexus tissue of the |
| | | | lateral ventricle removed from the brain of a 57-year-old Caucasian male, who died |
| | | | from a cerebrovascular accident. Patient history included Huntington's disease and |
| | | | emphysema. |
| 211 | 5573394 | TLYMNOT08 | Library was constructed using RNA isolated from anergic allogenic T-lymphocyte tissue |
| | | | removed from an adult (40-50-year-old) Caucasian male. The cells were incubated for 3 |
| | | | days in the presence of OKT3 mAb (1 microgram/mlOKT3) and 5% human serum. |
| 212 | 5850840 | FIBAUNT02 | Library was constructed using RNA isolated from untreated aortic adventitial |
| | | | fibroblasts removed from a 65-year-old Caucasian female. |
| 213 | 5942936 | COLADIT05 | Library was constructed using RNA isolated from diseased ascending colon tissue |
| | | | removed from a 32-year-old Caucasian male during a total intra-abdominal colectomy, |
| | | | abdominal-perineal rectal resection, and temporary ileostomy. Pathology indicated |
| | | | chronic ulcerative colitis extending in a continuous fashion from the mid-portion of |
| | | | the ascending colon to the rectum. This was characterized by crypt abscess formation |
| | | | and inflammation confined to the mucosa and submucosa. The terminal ileum exhibited |
| | | | ileitis and the rectal mucosa showed crypt abscess formation. The patient presented |
| | | | with ulcerative colitis and blood in the stools. Patient history included tobacco use. |
| | | | Patient medications included Imuran, prednisone, sulfasalazine, and azathioprine. |
| | | | Family history included ulcerative colitis, malignant breast neoplasm and acute |
| | | | myocardial infarction. |
| 214 | 5951431 | LIVRTUN04 | This normalized library was constructed from 1.72 million independent clones from an |
| | | | untreated C3A liver tumor library. C3A is a derivative of Hep G2, a cell line derived |
| | | | from a hepatoblastoma removed from a 15-year-old Caucasian male. The library was |
| | | | normalized in two rounds using conditions adapted from Soares et al., PNAS (1994) |
| | | | 91: 9228-9232 and Bonaldo et al., Genome Research 6 (1996): 791, except that a |
| | | | significantly longer (48 hours/round) reannealing hybridization was used. |

TABLE 5

| Program | Description | Reference | Parameter Threshold |
|---|---|---|---|
| ABI | A program that removes vector sequences and | Applied Biosystems, Foster City, CA. | |
| FACT- | masks ambiguous bases in nucleic acid sequences. | | |
| URA | | | |
| ABI/ | A Fast Data Finder useful in comparing and | Applied Biosystems, Foster City, CA; | Mismatch < 50% |
| PARA- | | | |
| CEL | | | |
| FDF | | | |
| | annotating amino acid or nucleic acid sequences. | Paracel Inc., Pasadena, CA. | |
| ABI Auto- | A program that assembles nucleic acid sequences. | Applied Biosystems, Foster City, CA. | |
| Assembler | | | |
| BLAST | A Basic Local Alignment Search Tool useful in | Altschul, S.F. et al. (1990) J. Mol. Biol. | ESTs: Probability value = 1.0E−8 |
| | sequence similarity search for amino acid and | 215:403410; Altschul, S.F. et al. (1997) | or less |
| | nucleic acid sequences. BLAST includes five | Nucleic Acids Res. 25:3389-3402. | Full Length |
| | sequences: Probability | | |
| | functions: blastp, blastn, blastx, tblastn, and tblastx. | | value = 1.0E−10 or less |
| FASTA | A Pearson and Lipruan algorithm that searches for | Pearson, W.R. and D.J. Lipruan (1988) Proc. | ESTs: fasta E value = 1.06E−6 |
| | similarity between a query sequence and a group of | Natl. Acad Sci. USA 85:2444-2448; Pearson, | Assembled ESTs: fasta |
| | sequences of the same type. FASTA comprises as | W.R. (1990) Methods Enzymol. 183:63-98; | Identity= 95% or greater and |
| | least five functions: fasta, tfasta, fastx, tfastx, and | and Smith, T.F. and M.S. Waterman (1981) | Match length = 200 bases or |
| | ssearch. | Adv. Appl. Math. 2:482-489. | greater; fastx E value = 1.0E−8 or |
| | | | less Full Length sequences: |
| | | | fastx score = 100 or greater |
| BLIMPS | A BLocks IMProved Searcher that matches a | Henikoff, S. and J.G. Henikoff (1991) Nucleic | Score = 1000 or greater; |
| | sequence against those in BLOCKS, PRINTS, | Acids Res. 19:6565-6572; Henikoff, J.G. and | Ratio of Score/Strength = 0.75 or |
| | DOMO, PRODOM, and PEAM databases to search | S. Henikoff (1996) Methods Enzymol. | larger; and, if applicable, |
| | for gene families, sequence homology, and structural | 266:88-105; and Attwood, T.K. et al. (1997) J. | Probability value = 1.0E−3 or less |
| | fingerprint regions. | Chem. Inf. Comput. Sci. 37:417-424. | |
| HMMER | An algorithm for searching a query sequence against | Krogh, A. et al. (1994) J. Mol. Biol. | Score = 10-50 bits for PFAM hits, |
| | hidden Markov model (HMM)-based databases of | 235:1501-1531; Sonnhammer, E.L.L. et al. | depending on individual protein |
| | protein family consensus sequences, such as PFAM. | (1988) Nucleic Acids Res. 26:320-322; | families |
| | | Durbin, R. et al. (1998) Our World View, in a | |
| | | Nutshell, Cambridge Univ. Press, pp. 1-350. | |
| Profile- | An algorithm that searches for structural and sequence | Gribskov, M. et al. (1988) CABIOS 4:61-66; | Normalized quality score ≦ GCG- |
| Scan | motifs in protein sequences that match | Gribskov, M. et al. (1989) Methods Enzymol. | specified “HIGH” value for that |
| | sequence patterns defined in Prosite. | 183:146-159; Bairoch, A. et al. (1997) | particular Prosite motif. |
| | | Nucleic Acids Res. 25:217-221. | Generally, score = 1.4-2.1. |
| Phred | A base-calling algorithm that examines automated | Ewing, B. et al. (1998) Genome Res. | |
| | sequencer traces with high sensitivity and probability. | 8:175-185; Ewing, B. and P. Green | |
| | | (1998) GenomeRes. 8:186-194. | |
| Phrap | A Phils Revised Assembly Program including | Smith, T.F. and M.S. Waterman (1981) Adv. | Score = 120 or greater; |
| | SWAT and CrossMatch, programs based on efficient | Appl. Math. 2:482-489; Smith, T.F. and M.S. | Match length = 56 or greater |
| | implementation of the Smith-Waterman algorithm, | Waterman (1981) J. Mol. Biol. 147:195-197; | |
| | useful in searching sequence homology and | and Green, P., University of Washington, | |
| | assembling DNA sequences | Seattle, WA. | |
| Consed | A graphical tool for viewing and | Gordon, D. et al. (1998) | |
| | editing Phrap assemblies. | Genome Res. 8:195-202. | |
| SPScan | A weight matrix analysis program that scans protein | Nielson, H. et al. (1997) Protein Engineering | Score = 3.5 or greater |
| | sequences for the presence of secretory signal peptides. | 10:1-6; Claverie, J.M. and S. Audic (1997) | |
| | | CABIOS 12:431-439. | |
| Motifs | A program that searches amino acid sequences for | Bairoch, A. et al. (1997) Nucleic | |
| | patterns that matched those defined in Prosite. | Acids Res. 25:217-221; | |
| | | Wisconsin Package Program Manual, | |
| | | version 9, page M51-59, | |
| | | Genetics Computer Group, Madison, WI. | |

What is claimed is:

1. An isolated polypeptide comprising an amino acid sequence selected from the group consisting of:
a) an amino acid sequence selected from the group consisting of SEQ ID NO:1-107,
b) a naturally occurring amino acid sequence having at least 90% sequence identity to an amino acid sequence selected from the group consisting of SEQ ID NO:1-107,
c) a biologically active fragment of an amino acid sequence selected from the group consisting of SEQ ID NO:1-107, and
d) an immunogenic fragment of an amino acid sequence selected from the group consisting of SEQ ID NO:1-107.

2. An isolated polypeptide of claim 1 selected from the group consisting of SEQ ID NO:1-107.

3. An isolated polynucleotide encoding a polypeptide of claim 1.

4. An isolated polynucleotide encoding a polypeptide of claim 2.

5. An isolated polynucleotide of claim 4 selected from the group consisting of SEQ ID NO:108-214.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method for producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. An isolated antibody which specifically binds to a polypeptide of claim 1.

11. An isolated polynucleotide comprising a polynucleotide sequence selected from the group consisting of:
a) a polynucleotide sequence selected from the group consisting of SEQ ID NO:108-214,
b) a naturally occurring polynucleotide sequence having at least 90% sequence identity to a polynucleotide sequence selected from the group consisting of SEQ ID NO:108-214,
c) a polynucleotide sequence complementary to a),
d) a polynucleotide sequence complementary to b), and
e) an RNA equivalent of a)-d).

12. An isolated polynucleotide comprising at least 60 contiguous nucleotides of a polynucleotide of claim 11.

13. A method for detecting a target polynucleotide in a sample, said target polynucleotide having a sequence of a polynucleotide of claim 11, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

14. A method of claim 13, wherein the probe comprises at least 60 contiguous nucleotides.

15. A method for detecting a target polynucleotide in a sample, said target polynucleotide having a sequence of a polynucleotide of claim 11, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

16. A composition comprising an effective amount of a polypeptide of claim 1 and a pharmaceutically acceptable excipient.

17. A composition of claim 16, wherein the polypeptide comprises an amino acid sequence selected from the group consisting of SEQ ID NO:1-107.

18. A method for treating a disease or condition associated with decreased expression of functional TRFX, comprising administering to a patient in need of such treatment the composition of claim 16.

19. A method for screening a compound for effectiveness as an agonist of a polypeptide of claim 1, the method comprising:
a) exposing a sample comprising a polypeptide of claim 1 to a compound, and
b) detecting agonist activity in the sample.

20. A composition comprising an agonist compound identified by a method of claim 19 and a pharmaceutically acceptable excipient.

21. A method for treating a disease or condition associated with decreased expression of functional BOX, comprising administering to a patient in need of such treatment a composition of claim 20.

22. A method for screening a compound for effectiveness as an antagonist of a polypeptide of claim 1, the method comprising:
a) exposing a sample comprising a polypeptide of claim 1 to a compound, and
b) detecting antagonist activity in the sample.

23. A composition comprising an antagonist compound identified by a method of claim 22 and a pharmaceutically acceptable excipient.

24. A method for treating a disease or condition associated with overexpression of functional TRFX, comprising administering to a patient in need of such treatment a composition of claim 23.

25. A method of screening for a compound that specifically binds to the polypeptide of claim 1, said method comprising the steps of:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

26. A method of screening for a compound that modulates the activity of the polypeptide of claim 1, said method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

27. A method for screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

28. A method for assessing toxicity of a test compound, said method comprising:
a) treating a biological sample containing nucleic acids with the test compound;
b) hybridizing the nucleic acids of the treated biological sample with a probe comprising at least 20 contiguous nucleotides of a polynucleotide of claim 11 under conditions whereby a specific hybridization complex is formed between said probe and a target polynucleotide in the biological sample, said target polynucleotide comprising a polynucleotide sequence of a polynucleotide of claim 11 or fragment thereof;
c) quantifying the amount of hybridization complex; and
d) comparing the amount of hybridization complex in the treated biological sample with the amount of hybridization complex in an untreated biological sample, wherein a difference in the amount of hybridization complex in the treated biological sample is indicative of toxicity of the test compound.
d) comparing the amount of hybridization complex in the treated biological sample with the amount of hybridization complex in an untreated biological sample wherein a difference in the amount of hybridization complex in the treated biological sample is indicative of toxicity of the test compound.

29. A method of claim 9, wherein the polypeptide has the sequence of SEQ ID NO:1.

30. A method of claim 9, wherein the polypeptide has the sequence of SEQ ID NO:2.

31. A method of claim 9, wherein the polypeptide has the sequence of SEQ ID NO:3.

32. A method of claim 9, wherein the polypeptide has the sequence of SEQ ID NO:4.

33. A method of claim 9, wherein the polypeptide has the sequence of SEQ ID NO:5.

34. A method of claim 9, wherein the polypeptide has the sequence of SEQ ID NO:6.

35. A method of claim 9, wherein the polypeptide has the sequence of SEQ ID NO:7.

36. A method of claim 9, wherein the polypeptide has the sequence of SEQ ID NO:8.

37. A method of claim 9, wherein the polypeptide has the sequence of SEQ ID NO:9.

38. A method of claim 9, wherein the polypeptide has the sequence of SEQ ID NO:10.

39. A method of claim 9, wherein the polypeptide has the sequence of SEQ ID NO:11.

40. A method of claim 9, wherein the polypeptide has the sequence of SEQ ID NO:12.

41. A method of claim 9, wherein the polypeptide has the sequence of SEQ ID NO:13.

42. A method of claim 9, wherein the polypeptide has the sequence of SEQ ID NO:14.

43. A method of claim 9, wherein the polypeptide has the sequence of SEQ ID NO:15.

44. A method of claim 9, wherein the polypeptide has the sequence of SEQ ID NO:16.

45. A method of claim 9, wherein the polypeptide has the sequence of SEQ ID NO:17.

46. A method of claim 9, wherein the polypeptide has the sequence of SEQ ID NO:18.

47. A method of claim 9, wherein the polypeptide has the sequence of SEQ ID NO:19.

48. A method of claim 9, wherein the polypeptide has the sequence of SEQ ID NO:20.

49. A method of claim 9, wherein the polypeptide has the sequence of SEQ ID NO:21.

50. A method of claim 9, wherein the polypeptide has the sequence of SEQ ID NO:22.

51. A method of claim 9, wherein the polypeptide has the sequence of SEQ ID NO:23.

52. A method of claim 9, wherein the polypeptide has the sequence of SEQ ID NO:24.

53. A method of claim 9, wherein the polypeptide has the sequence of SEQ ID NO:25.

54. A method of claim 9, wherein the polypeptide has the sequence of SEQ ID NO:26.

55. A method of claim 9, wherein the polypeptide has the sequence of SEQ ID NO:27.

56. A method of claim 9, wherein the polypeptide has the sequence of SEQ ID NO:28.

57. A method of claim 9, wherein the polypeptide has the sequence of SEQ ID NO:29.

58. A method of claim 9, wherein the polypeptide has the sequence of SEQ ID NO:30.

59. A method of claim 9, wherein the polypeptide has the sequence of SEQ ID NO:31.

60. A method of claim 9, wherein the polypeptide has the sequence of SEQ ID NO:32.

61. A method of claim 9, wherein the polypeptide has the sequence of SEQ ID NO:33.

62. A method of claim 9, wherein the polypeptide has the sequence of SEQ ID NO:34.

63. A method of claim 9, wherein the polypeptide has the sequence of SEQ ID NO:35.

64. A method of claim 9, wherein the polypeptide has the sequence of SEQ ID NO:36.

65. A method of claim 9, wherein the polypeptide has the sequence of SEQ ID NO:37.

66. A method of claim 9, wherein the polypeptide has the sequence of SEQ ID NO:38.

67. A method of claim 9, wherein the polypeptide has the sequence of SEQ ID NO:39.

68. A method of claim 9, wherein the polypeptide has the sequence of SEQ ID NO:40.

69. A method of claim 9, wherein the polypeptide has the sequence of SEQ ID NO:41.

70. A method of claim 9, wherein the polypeptide has the sequence of SEQ ID NO:42.

71. A method of claim 9, wherein the polypeptide has the sequence of SEQ ID NO:43.

72. A method of claim 9, wherein the polypeptide has the sequence of SEQ ID NO:44.

73. A method of claim 9, wherein the polypeptide has the sequence of SEQ ID NO:45.

74. A method of claim 9, wherein the polypeptide has the sequence of SEQ ID NO:46.

75. A method of claim 9, wherein the polypeptide has the sequence of SEQ ID NO:47.

76. A method of claim 9, wherein the polypeptide has the sequence of SEQ ID NO:48.

77. A method of claim 9, wherein the polypeptide has the sequence of SEQ ID NO:49.

78. A method of claim 9, wherein the polypeptide has the sequence of SEQ ID NO:50.

79. A method of claim 9, wherein the polypeptide has the sequence of SEQ ID NO:51.

80. A method of claim 9, wherein the polypeptide has the sequence of SEQ ID NO:52.

81. A method of claim 9, wherein the polypeptide has the sequence of SEQ ID NO:53.

82. A method of claim 9, wherein the polypeptide has the sequence of SEQ ID NO:54.

83. A method of claim 9, wherein the polypeptide has the sequence of SEQ ID NO:55.

84. A method of claim 9, wherein the polypeptide has the sequence of SEQ ID NO:56.

85. A method of claim 9, wherein the polypeptide has the sequence of SEQ ID NO:57.

86. A method of claim 9, wherein the polypeptide has the sequence of SEQ ID NO:58.

87. A method of claim 9, wherein the polypeptide has the sequence of SEQ ID NO:59.

88. A method of claim 9, wherein the polypeptide has the sequence of SEQ ID NO:60.

89. A method of claim 9, wherein the polypeptide has the sequence of SEQ ID NO:61.

90. A method of claim 9, wherein the polypeptide has the sequence of SEQ ID NO:62.

91. A method of claim 9, wherein the polypeptide has the sequence of SEQ ID NO:63.

92. A method of claim 9, wherein the polypeptide has the sequence of SEQ ID NO:64.

93. A method of claim 9, wherein the polypeptide has the sequence of SEQ ID NO:65.

94. A method of claim 9, wherein the polypeptide has the sequence of SEQ ID NO:66.

95. A method of claim 9, wherein the polypeptide has the sequence of SEQ ID NO:67.

96. A method of claim 9, wherein the polypeptide has the sequence of SEQ ID NO:68.

97. A method of claim 9, wherein the polypeptide has the sequence of SEQ ID NO:69.

98. A method of claim 9, wherein the polypeptide has the sequence of SEQ ID NO:70.

99. A method of claim 9, wherein the polypeptide has the sequence of SEQ ID NO:71.

100. A method of claim 9, wherein the polypeptide has the sequence of SEQ ID NO:72.

101. A method of claim 9, wherein the polypeptide has the sequence of SEQ ID NO:73.

102. A method of claim 9, wherein the polypeptide has the sequence of SEQ ID NO:74.

103. A method of claim 9, wherein the polypeptide has the sequence of SEQ ID NO:75.

104. A method of claim 9, wherein the polypeptide has the sequence of SEQ ID NO:76.

105. A method of claim 9, wherein the polypeptide has the sequence of SEQ ID NO:77.

106. A method of claim 9, wherein the polypeptide has the sequence of SEQ ID NO:78.

107. A method of claim 9, wherein the polypeptide has the sequence of SEQ ID NO:79.

108. A method of claim 9, wherein the polypeptide has the sequence of SEQ ID NO:80.

109. A method of claim 9, wherein the polypeptide has the sequence of SEQ ID NO:81.

110. A method of claim 9, wherein the polypeptide has the sequence of SEQ ID NO:82.

111. A method of claim 9, wherein the polypeptide has the sequence of SEQ ID NO:83.

112. A method of claim 9, wherein the polypeptide has the sequence of SEQ ID NO:84.

113. A method of claim 9, wherein the polypeptide has the sequence of SEQ ID NO:85.

114. A method of claim 9, wherein the polypeptide has the sequence of SEQ ID NO:86.

115. A method of claim 9, wherein the polypeptide has the sequence of SEQ ID NO:87.

116. A method of claim 9, wherein the polypeptide has the sequence of SEQ ID NO:88.

117. A method of claim 9, wherein the polypeptide has the sequence of SEQ ID NO:89.

118. A method of claim 9, wherein the polypeptide has the sequence of SEQ ID NO:90.

119. A method of claim 9, wherein the polypeptide has the sequence of SEQ ID NO:91.

120. A method of claim 9, wherein the polypeptide has the sequence of SEQ ID NO:92.

121. A method of claim 9, wherein the polypeptide has the sequence of SEQ ID NO:93.

122. A method of claim 9, wherein the polypeptide has the sequence of SEQ ID NO:94.

123. A method of claim 9, wherein the polypeptide has the sequence of SEQ ID NO:95.

124. A method of claim 9, wherein the polypeptide has the sequence of SEQ ID NO:96.

125. A method of claim 9, wherein the polypeptide has the sequence of SEQ ID NO:97.

126. A method of claim 9, wherein the polypeptide has the sequence of SEQ ID NO:98.

127. A method of claim 9, wherein the polypeptide has the sequence of SEQ ID NO:99.

128. A method of claim 9, wherein the polypeptide has the sequence of SEQ ID NO:100.

129. A method of claim 9, wherein the polypeptide has the sequence of SEQ ID NO:101.

130. A method of claim 9, wherein the polypeptide has the sequence of SEQ ID NO:102.

131. A method of claim 9, wherein the polypeptide has the sequence of SEQ ID NO:103.

132. A method of claim 9, wherein the polypeptide has the sequence of SEQ ID NO:104.

133. A method of claim 9, wherein the polypeptide has the sequence of SEQ ID NO:105.

134. A method of claim 9, wherein the polypeptide has the sequence of SEQ ID NO:106.

135. A method of claim 9, wherein the polypeptide has the sequence of SEQ ID NO:107.

136. A diagnostic test for a condition or disease associated with the expression of human transcription factors (TRFX) in a biological sample comprising the steps of:
a) combining the biological sample with an antibody of claim 10, under conditions suitable for the antibody to bind the polypeptide and form an antibody:polypeptide complex; and
b) detecting the complex wherein the presence of the complex correlates with the presence of the polypeptide in the biological sample.

137. The antibody of claim 10, wherein the antibody is:
a) a chimeric antibody,
b) a single chain antibody,
c) a Fab fragment,
d) a F(ab′)₂ fragment, or
e) a humanized antibody.

138. A composition comprising an antibody of claim 10 and an acceptable excipient.

139. A method of diagnosing a condition or disease associated with the expression of human transcription factors (TRFX) in a subject, comprising administering to said subject an effective amount of the composition of claim 138.

140. A composition of claim 138, wherein the antibody is labeled.

141. A method of diagnosing a condition or disease associated with the expression of human transcription factors (TRFX) in a subject, comprising administering to said subject an effective amount of the composition of claim 140.

142. A method of preparing a polyclonal antibody with the specificity of the antibody of claim 10 comprising:
a) immunizing an animal with a polypeptide having an amino acid sequence selected from the group consisting of SEQ ID NO:1-107 or an immunogenic fragment thereof, under conditions to elicit an antibody response;
b) isolating antibodies from said animal; and
c) screening the isolated antibodies with the polypeptide, thereby identifying a polyclonal antibody which binds specifically to a polypeptide having an amino acid sequence selected from the group consisting of SEQ ID NO:1-107.

143. An antibody produced by a method of claim 142.

144. A composition comprising the antibody of claim 143 and a suitable carrier.

145. A method of making a monoclonal antibody with the specificity of the antibody of claim 10 comprising:
a) immunizing an animal with a polypeptide having an amino acid sequence selected from the group consisting of SEQ ID NO:1-107 or an immunogenic fragment thereof, under conditions to elicit an antibody response;
b) isolating antibody producing cells from the animal;
c) fusing the antibody producing cells with immortalized cells to form monoclonal antibody-producing hybridoma cells;
d) culturing the hybridoma cells; and
e) isolating from the culture monoclonal antibody which binds specifically to a polypeptide having an amino acid sequence selected from the group consisting of SEQ ID NO:1-107.

146. A monoclonal antibody produced by a method of claim 145.

147. A composition comprising the antibody of claim 146 and a suitable carrier.

148. The antibody of claim 10, wherein the antibody is produced by screening a Fab expression library.

149. The antibody of claim 10, wherein the antibody is produced by screening a recombinant immunoglobulin library.

150. A method for detecting a polypeptide having an amino acid sequence selected from the group consisting of SEQ ID NO:1-107 in a sample, comprising the steps of:
a) incubating the antibody of claim 10 with a sample under conditions to allow specific binding of the antibody and the polypeptide; and
b) detecting specific binding, wherein specific binding indicates the presence of a polypeptide having an amino acid sequence selected from the group consisting of SEQ ID NO:1-107 in the sample.

151. A method of purifying a polypeptide having an amino acid sequence selected from the group consisting of SEQ ID NO:1-107 from a sample, the method comprising:
a) incubating the antibody of claim 10 with a sample under conditions to allow specific binding of the antibody and the polypeptide; and
b) separating the antibody from the sample and obtaining the purified polypeptide having an amino acid sequence selected from the group consisting of SEQ ID NO:1-107.

152. A microarray wherein at least one element of the microarray is a polynucleotide of claim 12.

153. A method for generating a transcript image of a sample which contains polynucleotides, the method comprising the steps of:
a) labeling the polynucleotides of the sample,
b) contacting the elements of the microarray of claim 152 with the labeled polynucleotides of the sample under conditions suitable for the formation of a hybridization complex, and
c) quantifying the expression of the polynucleotides in the sample.

154. An array comprising different nucleotide molecules affixed in distinct physical locations on a solid substrate, wherein at least one of said nucleotide molecules comprises a first oligonucleotide or polynucleotide sequence specifically hybridizable with at least 30 contiguous nucleotides of a target polynucleotide, said target polynucleotide having a sequence of claim 11.

155. An array of claim 154, wherein said first oligonucleotide or polynucleotide sequence is completely complementary to at least 30 contiguous nucleotides of said target polynucleotide.

156. An array of claim 154, wherein said first oligonucleotide or polynucleotide sequence is completely complementary to at least 60 contiguous nucleotides of said target polynucleotide.

157. An array of claim 154, which is a microarray.

158. An array of claim 154, further comprising said target polynucleotide hybridized to said first oligonucleotide or polynucleotide.

159. An array of claim 154, wherein a linker joins at least one of said nucleotide molecules to said solid substrate.

160. An array of claim 154, wherein each distinct physical location on the substrate contains multiple nucleotide molecules having the same sequence, and each distinct physical location on the substrate contains nucleotide molecules having a sequence which differs from the sequence of nucleotide molecules at another physical location on the substrate.

161. A polypeptide of claim 1, comprising the amino acid sequence of SEQ ID NO:1.

162. A polypeptide of claim 1, comprising the amino acid sequence of SEQ ID NO:2.

163. A polypeptide of claim 1, comprising the amino acid sequence of SEQ ID NO:3.

164. A polypeptide of claim 1, comprising the amino acid sequence of SEQ ID NO:4.

165. A polypeptide of claim 1, comprising the amino acid sequence of SEQ ID NO:5.

166. A polypeptide of claim 1, comprising the amino acid sequence of SEQ ID NO:6.

167. A polypeptide of claim 1, comprising the amino acid sequence of SEQ ID NO:7.

168. A polypeptide of claim 1, comprising the amino acid sequence of SEQ ID NO:8.

169. A polypeptide of claim 1, comprising the amino acid sequence of SEQ ID NO:9.

170. A polypeptide of claim 1, comprising the amino acid sequence of SEQ ID NO:10.

171. A polypeptide of claim 1, comprising the amino acid sequence of SEQ ID NO:11.

172. A polypeptide of claim 1, comprising the amino acid sequence of SEQ ID NO:12.

173. A polypeptide of claim 1, comprising the amino acid sequence of SEQ ID NO:13.

174. A polypeptide of claim 1, comprising the amino acid sequence of SEQ ID NO:14.

175. A polypeptide of claim 1, comprising the amino acid sequence of SEQ ID NO:15.

176. A polypeptide of claim 1, comprising the amino acid sequence of SEQ ID NO:16.

177. A polypeptide of claim 1, comprising the amino acid sequence of SEQ ID NO:17.

178. A polypeptide of claim 1, comprising the amino acid sequence of SEQ ID NO:18.

179. A polypeptide of claim 1, comprising the amino acid sequence of SEQ ID NO:19.

180. A polypeptide of claim 1, comprising the amino acid sequence of SEQ ID NO:20.

181. A polypeptide of claim 1, comprising the amino acid sequence of SEQ ID NO:21.

182. A polypeptide of claim 1, comprising the amino acid sequence of SEQ ID NO:22.

183. A polypeptide of claim 1, comprising the amino acid sequence of SEQ ID NO:23.

184. A polypeptide of claim 1, comprising the amino acid sequence of SEQ ID NO:24.

185. A polypeptide of claim 1, comprising the amino acid sequence of SEQ ID NO:25.

186. A polypeptide of claim 1, comprising the amino acid sequence of SEQ ID NO:26.

187. A polypeptide of claim 1, comprising the amino acid sequence of SEQ ID NO:27.

188. A polypeptide of claim 1, comprising the amino acid sequence of SEQ ID NO:28.

189. A polypeptide of claim 1, comprising the amino acid sequence of SEQ ID NO:29.

190. A polypeptide of claim 1, comprising the amino acid sequence of SEQ ID NO:30.

191. A polypeptide of claim 1, comprising the amino acid sequence of SEQ ID NO:31.

192. A polypeptide of claim 1, comprising the amino acid sequence of SEQ ID NO:32.

193. A polypeptide of claim 1, comprising the amino acid sequence of SEQ ID NO:33.

194. A polypeptide of claim 1, comprising the amino acid sequence of SEQ ID NO:34.

195. A polypeptide of claim 1, comprising the amino acid sequence of SEQ ID NO:35.

196. A polypeptide of claim 1, comprising the amino acid sequence of SEQ ID NO:36.

197. A polypeptide of claim 1, comprising the amino acid sequence of SEQ ID NO:37.

198. A polypeptide of claim 1, comprising the amino acid sequence of SEQ ID NO:38.

199. A polypeptide of claim 1, comprising the amino acid sequence of SEQ ID NO:39.

200. A polypeptide of claim 1, comprising the amino acid sequence of SEQ ID NO:40.

201. A polypeptide of claim 1, comprising the amino acid sequence of SEQ ID NO:41.

202. A polypeptide of claim 1, comprising the amino acid sequence of SEQ ID NO:42.

203. A polypeptide of claim 1, comprising the amino acid sequence of SEQ ID NO:43.

204. A polypeptide of claim 1, comprising the amino acid sequence of SEQ ID NO:44.

205. A polypeptide of claim 1, comprising the amino acid sequence of SEQ ID NO:45.

206. A polypeptide of claim 1, comprising the amino acid sequence of SEQ ID NO:46.

207. A polypeptide of claim 1, comprising the amino acid sequence of SEQ ID NO:47.

208. A polypeptide of claim 1, comprising the amino acid sequence of SEQ ID NO:48.

209. A polypeptide of claim 1, comprising the amino acid sequence of SEQ ID NO:49.

210. A polypeptide of claim 1, comprising the amino acid sequence of SEQ ID NO:50.

211. A polypeptide of claim 1, comprising the amino acid sequence of SEQ ID NO:51.

212. A polypeptide of claim 1, comprising the amino acid sequence of SEQ ID NO:52.

213. A polypeptide of claim 1, comprising the amino acid sequence of SEQ ID NO:53.

214. A polypeptide of claim 1, comprising the amino acid sequence of SEQ ID NO:54.

215. A polypeptide of claim 1, comprising the amino acid sequence of SEQ ID NO:55.

216. A polypeptide of claim 1, comprising the amino acid sequence of SEQ ID NO:56.

217. A polypeptide of claim 1, comprising the amino acid sequence of SEQ ID NO:57.

218. A polypeptide of claim 1, comprising the amino acid sequence of SEQ ID NO:58.

219. A polypeptide of claim 1, comprising the amino acid sequence of SEQ ID NO:59.

220. A polypeptide of claim 1, comprising the amino acid sequence of SEQ ID NO:60.

221. A polypeptide of claim 1, comprising the amino acid sequence of SEQ ID NO:61.

222. A polypeptide of claim 1, comprising the amino acid sequence of SEQ ID NO:62.

223. A polypeptide of claim 1, comprising the amino acid sequence of SEQ ID NO:63.

224. A polypeptide of claim 1, comprising the amino acid sequence of SEQ ID NO:64.

225. A polypeptide of claim 1, comprising the amino acid sequence of SEQ ID NO:65.

226. A polypeptide of claim 1, comprising the amino acid sequence of SEQ ID NO:66.

227. A polypeptide of claim 1, comprising the amino acid sequence of SEQ ID NO:67.

228. A polypeptide of claim 1, comprising the amino acid sequence of SEQ ID NO:68.

229. A polypeptide of claim 1, comprising the amino acid sequence of SEQ ID NO:69.

230. A polypeptide of claim 1, comprising the amino acid sequence of SEQ ID NO:70.

231. A polypeptide of claim 1, comprising the amino acid sequence of SEQ ID NO:71.

232. A polypeptide of claim 1, comprising the amino acid sequence of SEQ ID NO:72.

233. A polypeptide of claim 1, comprising the amino acid sequence of SEQ ID NO:73.

234. A polypeptide of claim 1, comprising the amino acid sequence of SEQ ID NO:74.

235. A polypeptide of claim 1, comprising the amino acid sequence of SEQ ID NO:75.

236. A polypeptide of claim 1, comprising the amino acid sequence of SEQ ID NO:76.

237. A polypeptide of claim 1, comprising the amino acid sequence of SEQ ID NO:77.

238. A polypeptide of claim 1, comprising the amino acid sequence of SEQ ID NO:78.

239. A polypeptide of claim 1, comprising the amino acid sequence of SEQ ID NO:79.

240. A polypeptide of claim 1, comprising the amino acid sequence of SEQ ID NO:80.

241. A polypeptide of claim 1, comprising the amino acid sequence of SEQ ID NO:81.

242. A polypeptide of claim 1, comprising the amino acid sequence of SEQ ID NO:82.

243. A polypeptide of claim 1, comprising the amino acid sequence of SEQ ID NO:83.

244. A polypeptide of claim 1, comprising the amino acid sequence of SEQ ID NO:84.

245. A polypeptide of claim 1, comprising the amino acid sequence of SEQ ID NO:85.

246. A polypeptide of claim 1, comprising the amino acid sequence of SEQ ID NO:86.

247. A polypeptide of claim 1, comprising the amino acid sequence of SEQ ID NO:87.

248. A polypeptide of claim 1, comprising the amino acid sequence of SEQ ID NO:88.

249. A polypeptide of claim 1, comprising the amino acid sequence of SEQ ID NO:89.

250. A polypeptide of claim 1, comprising the amino acid sequence of SEQ ID NO:90.

251. A polypeptide of claim 1, comprising the amino acid sequence of SEQ ID NO:91.

252. A polypeptide of claim 1, comprising the amino acid sequence of SEQ ID NO:92.

253. A polypeptide of claim 1, comprising the amino acid sequence of SEQ ID NO:93.

254. A polypeptide of claim 1, comprising the amino acid sequence of SEQ ID NO:94.

255. A polypeptide of claim 1, comprising the amino acid sequence of SEQ ID NO:95.

256. A polypeptide of claim 1, comprising the amino acid sequence of SEQ ID NO:96.

257. A polypeptide of claim 1, comprising the amino acid sequence of SEQ ID NO:97.

258. A polypeptide of claim 1, comprising the amino acid sequence of SEQ ID NO:98.

259. A polypeptide of claim 1, comprising the amino acid sequence of SEQ ID NO:99.

260. A polypeptide of claim 1, comprising the amino acid sequence of SEQ ID NO:100.

261. A polypeptide of claim 1, comprising the amino acid sequence of SEQ ID NO:101.

262. A polypeptide of claim 1, comprising the amino acid sequence of SEQ ID NO:102.

263. A polypeptide of claim 1, comprising the amino acid sequence of SEQ ID NO:103.

264. A polypeptide of claim 1, comprising the amino acid sequence of SEQ ID NO:104.

265. A polypeptide of claim 1, comprising the amino acid sequence of SEQ ID NO:105.

266. A polypeptide of claim 1, comprising the amino acid sequence of SEQ ID NO:106.

267. A polypeptide of claim 1, comprising the amino acid sequence of SEQ ID NO:107.

268. A polynucleotide of claim 11, comprising the polynucleotide sequence of SEQ ID NO:108.

269. A polynucleotide of claim 11, comprising the polynucleotide sequence of SEQ ID NO:109.

270. A polynucleotide of claim 11, comprising the polynucleotide sequence of SEQ ID NO:110.

271. A polynucleotide of claim 11, comprising the polynucleotide sequence of SEQ ID NO:111.

272. A polynucleotide of claim 11, comprising the polynucleotide sequence of SEQ ID NO:112.

273. A polynucleotide of claim 11, comprising the polynucleotide sequence of SEQ ID NO:113.

274. A polynucleotide of claim 11, comprising the polynucleotide sequence of SEQ ID NO:114.

275. A polynucleotide of claim 11, comprising the polynucleotide sequence of SEQ ID NO:115.

276. A polynucleotide of claim 11, comprising the polynucleotide sequence of SEQ ID NO:116.

277. A polynucleotide of claim 11, comprising the polynucleotide sequence of SEQ ID NO:117.

278. A polynucleotide of claim 11, comprising the polynucleotide sequence of SEQ ID NO:118.

279. A polynucleotide of claim 11, comprising the polynucleotide sequence of SEQ ID NO:119.

280. A polynucleotide of claim 11, comprising the polynucleotide sequence of SEQ ID NO:120.

281. A polynucleotide of claim 11, comprising the polynucleotide sequence of SEQ ID NO:121.

282. A polynucleotide of claim 11, comprising the polynucleotide sequence of SEQ ID NO:122.

283. A polynucleotide of claim 11, comprising the polynucleotide sequence of SEQ ID NO:123.

284. A polynucleotide of claim 11, comprising the polynucleotide sequence of SEQ ID NO:124.

285. A polynucleotide of claim 11, comprising the polynucleotide sequence of SEQ ID NO:125.

286. A polynucleotide of claim 11, comprising the polynucleotide sequence of SEQ ID NO:126.

287. A polynucleotide of claim 11, comprising the polynucleotide sequence of SEQ ID NO:127.

288. A polynucleotide of claim 11, comprising the polynucleotide sequence of SEQ ID NO:128.

289. A polynucleotide of claim 11, comprising the polynucleotide sequence of SEQ ID NO:129.

290. A polynucleotide of claim 11, comprising the polynucleotide sequence of SEQ ID NO:130.

291. A polynucleotide of claim 11, comprising the polynucleotide sequence of SEQ ID NO:131.

292. A polynucleotide of claim 11, comprising the polynucleotide sequence of SEQ ID NO:132.

293. A polynucleotide of claim 11, comprising the polynucleotide sequence of SEQ ID NO:133.

294. A polynucleotide of claim 11, comprising the polynucleotide sequence of SEQ ID NO:134.

295. A polynucleotide of claim 11, comprising the polynucleotide sequence of SEQ ID NO:135.

296. A polynucleotide of claim 11, comprising the polynucleotide sequence of SEQ ID NO:136.

297. A polynucleotide of claim 11, comprising the polynucleotide sequence of SEQ ID NO:137.

298. A polynucleotide of claim 11, comprising the polynucleotide sequence of SEQ ID NO:138.

299. A polynucleotide of claim 11, comprising the polynucleotide sequence of SEQ ID NO:139.

300. A polynucleotide of claim 11, comprising the polynucleotide sequence of SEQ ID NO:140.

301. A polynucleotide of claim 11, comprising the polynucleotide sequence of SEQ ID NO:141.

302. A polynucleotide of claim 11, comprising the polynucleotide sequence of SEQ ID NO:142.

303. A polynucleotide of claim 11, comprising the polynucleotide sequence of SEQ ID NO:143.

304. A polynucleotide of claim 11, comprising the polynucleotide sequence of SEQ ID NO:144.

305. A polynucleotide of claim 11, comprising the polynucleotide sequence of SEQ ID NO:145.

306. A polynucleotide of claim 11, comprising the polynucleotide sequence of SEQ ID NO:146.

307. A polynucleotide of claim 11, comprising the polynucleotide sequence of SEQ ID NO:147.

308. A polynucleotide of claim 11, comprising the polynucleotide sequence of SEQ ID NO:148.

309. A polynucleotide of claim 11, comprising the polynucleotide sequence of SEQ ID NO:149.

310. A polynucleotide of claim 11, comprising the polynucleotide sequence of SEQ ID NO:150.

311. A polynucleotide of claim 11, comprising the polynucleotide sequence of SEQ ID NO:151.

312. A polynucleotide of claim 11, comprising the polynucleotide sequence of SEQ ID NO:152.

313. A polynucleotide of claim 11, comprising the polynucleotide sequence of SEQ ID NO:153.

314. A polynucleotide of claim 11, comprising the polynucleotide sequence of SEQ ID NO:154.

315. A polynucleotide of claim 11, comprising the polynucleotide sequence of SEQ ID NO:155.

316. A polynucleotide of claim 11, comprising the polynucleotide sequence of SEQ ID NO:156.

317. A polynucleotide of claim 11, comprising the polynucleotide sequence of SEQ ID NO:157.

318. A polynucleotide of claim 11, comprising the polynucleotide sequence of SEQ ID NO:158.

319. A polynucleotide of claim 11, comprising the polynucleotide sequence of SEQ ID NO:159.

320. A polynucleotide of claim 11, comprising the polynucleotide sequence of SEQ ID NO:160.

321. A polynucleotide of claim 11, comprising the polynucleotide sequence of SEQ ID NO:161.

322. A polynucleotide of claim 11, comprising the polynucleotide sequence of SEQ ID NO:162.

323. A polynucleotide of claim 11, comprising the polynucleotide sequence of SEQ ID NO:163.

324. A polynucleotide of claim 11, comprising the polynucleotide sequence of SEQ ID NO:164.

325. A polynucleotide of claim 11, comprising the polynucleotide sequence of SEQ ID NO:165.

326. A polynucleotide of claim 11, comprising the polynucleotide sequence of SEQ ID NO:166.

327. A polynucleotide of claim 11, comprising the polynucleotide sequence of SEQ ID NO:167.

328. A polynucleotide of claim 11, comprising the polynucleotide sequence of SEQ ID NO:168.

329. A polynucleotide of claim 11, comprising the polynucleotide sequence of SEQ ID NO:169.

330. A polynucleotide of claim 11, comprising the polynucleotide sequence of SEQ ID NO:170.

331. A polynucleotide of claim 11, comprising the polynucleotide sequence of SEQ ID NO:171.

332. A polynucleotide of claim 11, comprising the polynucleotide sequence of SEQ ID NO:172.

333. A polynucleotide of claim 11, comprising the polynucleotide sequence of SEQ ID NO:173.

334. A polynucleotide of claim 11, comprising the polynucleotide sequence of SEQ ID NO:174.

335. A polynucleotide of claim 11, comprising the polynucleotide sequence of SEQ ID NO:175.

336. A polynucleotide of claim 11, comprising the polynucleotide sequence of SEQ ID NO:176.

337. A polynucleotide of claim 11, comprising the polynucleotide sequence of SEQ ID NO:177.

338. A polynucleotide of claim 11, comprising the polynucleotide sequence of SEQ ID NO:178.

339. A polynucleotide of claim 11, comprising the polynucleotide sequence of SEQ ID NO:179.

340. A polynucleotide of claim 11, comprising the polynucleotide sequence of SEQ ID NO:180.

341. A polynucleotide of claim 11, comprising the polynucleotide sequence of SEQ ID NO:181.

342. A polynucleotide of claim 11, comprising the polynucleotide sequence of SEQ ID NO:182.

343. A polynucleotide of claim 11, comprising the polynucleotide sequence of SEQ ID NO:183.

344. A polynucleotide of claim 11, comprising the polynucleotide sequence of SEQ ID NO:184.

345. A polynucleotide of claim 11, comprising the polynucleotide sequence of SEQ ID NO:185.

346. A polynucleotide of claim 11, comprising the polynucleotide sequence of SEQ ID NO:186.

347. A polynucleotide of claim 11, comprising the polynucleotide sequence of SEQ ID NO:187.

348. A polynucleotide of claim 11, comprising the polynucleotide sequence of SEQ ID NO:188.

349. A polynucleotide of claim 11, comprising the polynucleotide sequence of SEQ ID NO:189.

350. A polynucleotide of claim 11, comprising the polynucleotide sequence of SEQ ID NO:190.

351. A polynucleotide of claim 11, comprising the polynucleotide sequence of SEQ ID NO:191.

352. A polynucleotide of claim 11, comprising the polynucleotide sequence of SEQ ID NO:192.

353. A polynucleotide of claim 11, comprising the polynucleotide sequence of SEQ ID NO:193.

354. A polynucleotide of claim 11, comprising the polynucleotide sequence of SEQ ID NO:194.

355. A polynucleotide of claim 11, comprising the polynucleotide sequence of SEQ ID NO:195.

356. A polynucleotide of claim 11, comprising the polynucleotide sequence of SEQ ID NO:196.

357. A polynucleotide of claim 11, comprising the polynucleotide sequence of SEQ ID NO:197.

358. A polynucleotide of claim 11, comprising the polynucleotide sequence of SEQ ID NO:198.

359. A polynucleotide of claim 11, comprising the polynucleotide sequence of SEQ ID NO:199.

360. A polynucleotide of claim 11, comprising the polynucleotide sequence of SEQ ID NO:200.

361. A polynucleotide of claim 11, comprising the polynucleotide sequence of SEQ ID NO:201.

362. A polynucleotide of claim 11, comprising the polynucleotide sequence of SEQ ID NO:202.

363. A polynucleotide of claim 11, comprising the polynucleotide sequence of SEQ ID NO:203.

364. A polynucleotide of claim 11, comprising the polynucleotide sequence of SEQ ID NO:204.

365. A polynucleotide of claim 11, comprising the polynucleotide sequence of SEQ ID NO:205.

366. A polynucleotide of claim 11, comprising the polynucleotide sequence of SEQ ID NO:206.

367. A polynucleotide of claim 11, comprising the polynucleotide sequence of SEQ ID NO:207.

368. A polynucleotide of claim 11, comprising the polynucleotide sequence of SEQ ID NO:208.

369. A polynucleotide of claim 11, comprising the polynucleotide sequence of SEQ ID NO:209.

370. A polynucleotide of claim 11, comprising the polynucleotide sequence of SEQ ID NO:210.

371. A polynucleotide of claim 11, comprising the polynucleotide sequence of SEQ ID NO:211.

372. A polynucleotide of claim 11, comprising the polynucleotide sequence of SEQ ID NO:212.

373. A polynucleotide of claim 11, comprising the polynucleotide sequence of SEQ ID NO:213.

374. A polynucleotide of claim 11, comprising the polynucleotide sequence of SEQ ID NO:214.
